(19) United States
(12) Patent Application Publication
(10) Pub. No.: US 2023/0381774 A1
(43) Pub. Date: Nov. 30, 2023

(54) MICROFLUDIC LAB-ON-CHIP DEVICE, MATRIX, SMALL MOLECULES AND THREE-DIMENSIONAL SPHEROIDS FOR CELL REPROGRAMMING

(71) Applicant: The Regents of the University of California, Oakland, CA (US)

(72) Inventors: Song Li, Beverly Hills, CA (US); Yang Song, Los Angeles, CA (US); Jennifer Soto, Santa Monica, CA (US); LeeAnn Li, Los Angeles, CA (US); Binru Chen, Los Angeles, CA (US); Yifan Wu, Los Angeles, CA (US)

(73) Assignee: The Regents of the University of California, Oakland, CA (US)

(21) Appl. No.: 18/315,370

(22) Filed: May 10, 2023

Related U.S. Application Data
(60) Provisional application No. 63/340,092, filed on May 10, 2022.

(51) Int. Cl. B01L 3/00

(52) U.S. Cl. CPC B01L 3/502707; B01L 2300/16

(57) ABSTRACT

Cellular reprogramming and gene editing represent major advancements in biology, and has wide applications in regenerative medicine, disease therapy and drug screening. However, low and variable efficiencies have created significant roadblocks to the full application of these technologies. The invention disclosed herein overcomes these roadblocks by providing optimized methods and systems that are useful in a variety of cellular engineering and gene editing methodologies, including for example methods designed to enhance the reprogramming of somatic cells into neural cells or pluripotent cells. The invention provides innovative microfluidic devices, chemical treatment, cell adhesion manipulation, and 3D spheroid culture to modulate epigenetic changes and significantly enhance cell reprogramming and gene editing; the genome-wide chromatin accessibility changes caused by cell nuclear deformation, 3D culture, decreased cell adhesions, and the reduction of intracellular tension can provide guidance for guided gene silencing, activation, insertion and/or editing.

### CROSS-REFERENCE TO RELATED APPLICATIONS

This application claims the benefit under 35 U.S.C. Section 119(e) of co-pending and U.S. Provisional Patent Application Ser. No. 63/340,092, filed on May 10, 2022 and entitled “MICROFLUDIC LAB-ON-CHIP DEVICE, SMALL MOLECULES AND THREE-DIMENSIONAL SPHEROIDS FOR CELL REPROGRAMMING” which application is incorporated by reference herein.

### TECHNICAL FIELD

The present invention relates to methods and materials for cellular reprogramming and gene editing.

### BACKGROUND OF THE INVENTION

Cell reprogramming is a process used for converting cells from one phenotype to another. Cell reprogramming enables the derivation of different cell types that are highly valuable for use in a variety of biomedical technologies including regenerative cell therapy, disease modeling and therapeutic discovery. While direct cell conversion provides a fast and direct method of generating desired cell types from somatic cells, a low conversion/reprogramming efficiency has limited the use of direct reprogramming strategies in therapeutic applications. A major barrier to overcome is the epigenetic state of the cell, regardless of the methods being used for cell reprogramming, including genetic engineering, chemical cocktails or biophysical approaches.

The epigenetic state of a cell is the “memory” of cell identity that controls the organization of chromatin and on/off state of phenotypic genes without altering DNA sequence, which can be regulated by histone modifications (e.g., methylation, acetylation) and DNA methylation. It has been shown that epigenetic modifications, such as histone methylation and acetylation, play an important role in cell reprogramming. Although the regulation of epigenetic state by biomolecules and chemicals have been widely studied, the roles of biophysical factors are not well understood. Recent studies have shown that biophysical signals can be transmitted through focal adhesions (FAs), cytoskeleton and nuclear lamina to modulate chromatin organization and epigenetic state, which may facilitate cell reprogramming. Here, we develop and present new approaches to enhance cell reprogramming by (1) mechanically deforming cell nucleus in microfluidic device, (2) reducing intracellular tension and/or reducing cell adhesion to an optimal level by using chemicals or matrix engineering, (3) engineering the mechanical properties of cell-adhesion substrates/matrix such as stiffness and viscoelasticity, and (4) culturing cells in three-dimensional (3D) spheroids.

There is a continuing need for cell reprograming methods and materials, particularly those capable of making cells that are useful in the treatment of injury and disease. Cell reprogramming is a process used for converting cells from one phenotype to another. Cell reprogramming enables the derivation of different cell types that are highly valuable for use in a variety of biomedical technologies including regenerative cell therapy, disease modeling and therapeutic discovery. While direct cell conversion provides a fast and direct method of generating desired cell types from somatic cells, a low conversion/reprogramming efficiency has limited the use of direct reprogramming strategies in therapeutic applications. A major barrier to overcome is the epigenetic state of the cell, regardless of the methods being used for cell reprogramming, including genetic engineering, chemical cocktails or biophysical approaches.

The epigenetic state of a cell is the “memory” of cell identity that controls the organization of chromatin and on/off state of phenotypic genes without altering DNA sequence, which can be regulated by histone modifications (e.g., methylation, acetylation) and DNA methylation. It has been shown that epigenetic modifications, such as histone methylation and acetylation, play an important role in cell reprogramming. Although the regulation of epigenetic state by biomolecules and chemicals have been widely studied, the roles of biophysical factors are not well understood. Recent studies have shown that biophysical signals can be transmitted through focal adhesions (FAs), cytoskeleton and nuclear lamina to modulate chromatin organization and epigenetic state, which may facilitate cell reprogramming. Here, we develop and present new approaches to enhance cell reprogramming by (1) mechanically deforming cell nucleus in microfluidic device, (2) reducing intracellular tension, (3) reducing cell adhesion, and (4) culturing cells in three-dimensional (3D) spheroids.

There is a continuing need for cell reprograming methods and materials, particularly those capable of making cells that are useful in the treatment of injury and disease. Our approaches will enable more efficient cell reprograming by regulating the epigenetic state of the cells via physical and chemical modulations of cell nucleus, intracellular tension, cell adhesions and cell-cell interactions.

### SUMMARY OF THE INVENTION

As discussed in detail below, it has been discovered that an innovative microfluidic device can be used to optimize a number of different methodologies for cell reprogramming and gene editing. In one illustration of this, microfluidic devices of the invention can be used to significantly enhance methods of cellular reprogramming. Cellular reprogramming represents a major advancement in biology, and has wide applications in regenerative medicine, disease modeling and drug screening. Despite the enormous potential of reprogramming technologies, low and variable efficiencies in cell reprogramming, in particular, during induced neuronal (iN) conversion, have created significant roadblocks to the full application of this technology. The invention disclosed herein overcomes these roadblocks by providing optimized methods and systems that are useful in a variety of cellular engineering methodologies, including for example methods designed to enhance direct iN cellular reprogramming.

Embodiments of the invention include high throughput microfluidic systems with microchannels having sizes and architectures (e.g., cell-type specific designs) that are selected to deform cell nuclei and induce chromatin reorganization in a manner that boosts the efficiency of a variety of methods for modulating the fate and function of mammalian cells including cellular reprogramming, transcriptional activation/suppression, gene addition, gene deletion, gene editing and the like. Such embodiments include, for example, a microfluidic cell culture/processing system comprising: an inlet reservoir configured to receive cells; an outlet reservoir to remove cells from the microfluidic system; and at least one channel coupling the inlet reservoir to the outlet reservoir. In such microfluidic processing systems, the at least one channel is configured so that selected mammalian cells contact the channel and experience cellular and/or nuclear deformation as the cells are moved from the inlet reservoir through the channel to the outlet reservoir. In illustrative working embodiments of the invention, the inlet reservoir, the outlet reservoir and the at least one channel are disposed on a polydimethylsiloxane device in a lab-on-a-chip configuration.

As discussed in the Examples below, we utilized size selected mammalian cells and microfluidic channels to induce a millisecond deformation of the cell nucleus of cells going through the channel, a deformation which caused the wrinkling and transient disassembly of the nuclear lamina, local detachment of lamina-associated domain of chromatin from nuclear lamina, and a decrease of histone methylation (H3K9me3) and DNA methylation. Moreover, we have discovered that such mechanical squeezing of cells results in global changes in chromatin, which then enhances associated efforts to modulate the fate and function of cells, for example the reprogramming of cells (e.g., reprogramming fibroblasts into neurons). This mechanopriming approach can further be used enhance other reprogramming processes such as turning macrophages into neurons and converting fibroblasts into induced pluripotent stem cells.

In view of the discovery that mechanopriming approaches using selected cells and channel sizes can enhance cellular reprogramming as well as a number of other genetic engineering processes, embodiments of the invention include methods of deforming cells in the microfluidic systems of the invention. For example, embodiments of the invention include a method of determining the size of the mammalian cell and further selecting a width/height and/or architecture of the at least one channel in the microfluidic cell culture system such that the mammalian cell contacts the channel and undergoes cellular and/or nuclear deformation as the cell moves from the inlet reservoir through the channel to the outlet reservoir, such that the cells are collected from the microfluidic cell culture system. In some embodiments of the invention, the aspect ratio of the channel width and height and/or the cross-section area ratio of the cell and microchannel is further selected. For example, certain embodiments of the invention selectively utilize channels having an aspect ratio between 0.2 and 1 (e.g., an aspect ratio of about 0.5). In addition, certain embodiments of the invention selectively utilize cells observed to exhibit a defined cross-section area ratio, such as a cross-section area ratio between 0.75 and 1.25 (e.g., a cross-section area ratio of about 1). Typically in these methods, the mammalian cells are selected to be mammalian cells in single cell suspension.

The microfluidic cell processing systems disclosed herein can be used to generate a wide variety of cells with high efficiency for applications such as tissue/organ regeneration, disease modeling/drug screening and disease therapies. For example, methods of the invention include those designed for: (1) high-efficiency conversion of any cell types (e.g., skin biopsy, blood cells) into induced pluripotent stem cells or any other cells (e.g., neurons, cardiomyocytes, pancreatic cells); (2) high-efficiency guided stem cell differentiation (e.g., stem cell differentiation into neurons, cardiomyocytes, pancreatic cells); (3) boosting the efficiency of gene therapy and gene introduction/transduction in vitro, e.g., creating CAR-macrophages, CAR-T cells with higher efficiency; and (4) reorganizing the chromatin to facilitate the rejuvenation of cells.

In certain embodiments of the methods invention, cells disposed in the microfluidic devices disclosed herein are manipulated under such culture conditions. In some embodiments of the invention, a microfluidic cell processing system of the invention is used to facilitate reprogramming and/or differentiation of mammalian cells. In some embodiments of the invention, a microfluidic cell culture system of the invention is used to boost the efficiency of gene editing and the like in mammalian cells. In certain embodiments, somatic cells are induced to reprogram into neuronal cells in this microfluidic cell culture system.

In Example 2, we show that a reduction of intracellular tension by action cytoskeleton disruption or FA inhibition to an optimal level can generally increase the chromatin accessibility to facilitate gene activation and cell reprogramming. This manipulation of intracellular tension and cell adhesion can be achieved by chemical compounds (e.g., inhibitor for actin-myosin interactions such as blebbistatin, focal adhesion kinase inhibitor PF573228, and any chemical compounds that interfere the assembly and dynamics of actin cytoskeleton and FAs), and/or cell adhesion substrates that reduce cell adhesion and intracellular tension to an optimal level by engineering the ionic property, ligand density, surface topography (e.g., micro/nano structure) and any other properties affecting cell adhesion.

In Example 5 and 6, we show that the mechanical properties of adhesion substrates such as stiffness and viscoelasticity have profound effects on chromatin accessibility and cell reprogramming. For example, an intermediate stiffness, rather than stiff or soft surfaces, results in highest cell reprogramming efficiency; viscoelastic property at low stiffness surface (e.g., 0.1-5 kPa) can further enhance cell reprogramming. Therefore, tailoring the stiffness and viscoelastic properties of materials can be used to modulate the epigenetic state of cells and thus the efficiency of reprogramming and gene editing.

In certain embodiments of the invention, the mammalian cells are configured as three-dimensional spheroids. In Example 4, we show that cell reprogramming in 3D spheroids has much higher efficiency than 2D surface and that the reprogramming appears to start from the surface of the spheroids. These effects are related to the 3D cell-cell interactions that are not present in 2D culture and the resulting epigenetic changes in 3D culture. Therefore, this 3D spheroid culture approach can be used to promote cell reprogramming and gene editing. The spheroid size can be 100-500 μm (cannot be too big; otherwise may cause cell death in the core of spheroids).

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Actin cytoskeleton and cell mechanical phenotype are altered during iN reprogramming. (A), Reprogramming protocol. Adult fibroblasts transduced with the reprogramming factors (BAM) were cultured in N3 medium with doxycycline (Dox) for 14 days before immunostaining and quantification. (B), Representative images show fluorescence micrograph of the actin network (phalloidin, red) and nucleus (DAPI, blue) in BAM-transduced fibroblasts at the indicated time points. Scale bars, 20 μm. ©, Density scatter plots show the elastic modulus as a function of cell diameter for BAM-transduced fibroblasts deforming through 9×10 μm constrictions at the indicated time points (day 0, n=211; day 1 n=257; day 3, n=253). Dots represent single-cell data. (D), Elastic modulus of BAM-transduced fibroblasts at the indicated time points (day 0, n=211; day 1 n=257; day 3, n=253) as derived by q-DC. Significance was determined by a one-way ANOVA and Tukey's multiple comparison test. €, Box plots illustrate the variation in elastic modulus of BAM- or GFP-transduced fibroblasts at the indicated time points as acquired using AFM, where GFP serves as a control. The number of biological replicates, n, was equal to 55 per condition. Significance determined by two-way ANOVA using Tukey's correction for multiple comparisons. Box plots show the ends at the quartiles, the median as a horizontal line in the box, the mean as a (+) symbol, and the whiskers extend from the minimum to maximum data point (*p<0.05,**p<0.01, ***p<0.001, ****p<0.0001).

FIG. 2. Ascl1 plays a dominant role in regulating cell stiffness and intracellular structures during iN reprogramming. For experiments in (A) and (B), fibroblasts were transduced with individual or various combinations of the transgenes and collected at the indicated time points for immunofluorescence analysis or AFM measurements, where non-transduced (NT) fibroblasts served as a control. (A), Representative immunofluorescent images show the actin network (phalloidin, red), focal adhesions (paxillin, green) and nucleus (DAPI, blue) in fibroblasts transduced with individual or various combinations of the transgenes at the indicated time points. Scale bar, 50 μm. (B), Box plots display the distribution of elastic modulus at the indicated time points as acquired using AFM (n=33 per condition). Significance determined by two-way ANOVA using Tukey's correction for multiple comparisons. (C), Genes within the listed gene ontology terms are up-regulated by Ascl1 overexpression compared to non-transduced fibroblasts. Box plots show the ends at the quartiles, the median as a horizontal line in the box, the mean as a (+) symbol, and the whiskers extend from the minimum to maximum data point (*p<0.05, **p<0.01, ***p<0.001, ****p<0.0001).

FIG. 3. Inhibition of cytoskeletal tension enhances iN conversion. (A), Representative fluorescent micrographs of iN cells generated from BAM-transduced fibroblasts in the absence and presence of 10 μM blebbistatin (denoted as Blebb in this and all subsequent figures). iN cells expressed TUBB3 and formed neural networks. Scale bar, 100 μM. (B), Reprogramming efficiency of fibroblasts transduced with BAM and cultured in absence and presence of varying concentrations of blebbistatin for 7 days, where DMSO served as a control (n=3). Significance was determined by a one-way ANOVA and Dunnett's multiple comparison test. (C), Reprogramming efficiency of fibroblasts transduced with Ascl1 and cultured in the absence and presence of 10 μM blebbistatin (n=6). Significance determined by two-tailed, unpaired t test. (D), Reprogramming efficiency of BAM-transduced fibroblasts treated with and without the ROCK inhibitor, Y-27632 [20 μM], for 7 days (n=3). Significance determined by two-tailed, unpaired t test. €, Reprogramming efficiency of fibroblasts transduced with BAM and cultured in the presence of 0.3 μM Nocodazole (Noc) for 4 days (n=3). Significance determined by two-tailed, unpaired t test. (F), Reprogramming efficiency of BAM-transduced fibroblasts cultured in the absence and presence of 10 μM blebbistatin, 1 μM Cytochalasin D (CytoD), and 0.05 μM Jasplakinolide (Jas) (n=3). Significance was determined by a one-way ANOVA and Dunnett's multiple comparison test. (G), Reprogramming efficiency of BAM-transduced fibroblasts treated with 10 μM blebbistatin during the early (i.e., days 1-5), mid (i.e., days 5-9), and late (i.e., days 9-13) phases of reprogramming (n=3). Significance determined by two-way ANOVA using Sidak's multiple comparison test. (H), Representative fluorescent images of TUBB3⁺ iN cells co-expressing mature neuronal markers, NeuN, MAP2 and synapsin. Scale bar, 100 μm. (I), Representative traces of spontaneous changes in membrane potential in response to current injection from iN cells obtained in the presence and absence of 10 μM blebbistatin. Bar graphs show mean±standard deviation (*p<0.05, **p<0.01, ***p<0.001, ****p<0.0001).

FIG. 4. Cytoskeletal tension inhibition regulates the expression of mesenchymal and neuronal markers in the early phase of reprogramming. (A), Images show fluorescence micrograph of the actin network (phalloidin, red), focal adhesions (paxillin, green) and nucleus (DAPI, blue) of fibroblasts were treated with 10 μM blebbistatin for 24 hours. Scale bar, 50 μm. (B), Immunofluorescent staining of calponin and αSMA in non-transduced fibroblasts after blebbistatin treatment for 24 hours. Scale bar, 100 μm. (C), qRT-PCR analysis of ACTA2 and CNN1 expression at day 3 from non-transduced (NT) and BAM-transduced fibroblasts cultured in the absence and presence of blebbistatin for 2 days (n=3). Expression level normalized to BAM-transduced fibroblasts treated with DMSO. Significance determined by one-way ANOVA and Sidak's multiple comparison test, compared to the corresponding DMSO condition for the same gene. (D), NT or BAM-transduced fibroblasts were treated with blebbistatin for 2 days, followed by Western blot analysis of mesenchymal markers, αSMA and calponin. GAPDH is shown as a loading control. €, qRT-PCR analysis of neuronal gene expression at day 5 from NT and BAM transduced fibroblasts cultured in the absence and presence of blebbistatin for 4 days (n=3). Expression level normalized to BAM-transduced fibroblasts treated with DMSO. Significance determined by two-tailed, unpaired t test, compared to the DMSO condition for transduced cells for the same gene. Bar graphs show mean±standard deviation (*p<0.05, **p<0.01, ***p<0.001, ****p<0.0001).

FIG. 5. Cytoskeleton disruption modulates the epigenetic state to promote iN reprogramming. (A), Representative images show the fluorescence intensity of histone modifications in non-transduced fibroblasts treated with 10 μM blebbistatin for 2 hours. Scale bar, 10 μm. (B), Quantification of histone acetyltransferase (HAT) activity in fibroblasts treated with DMSO or 10 μM blebbistatin for 2 hours (n=5). Significance determined by two-tailed, unpaired t test, compared to DMSO condition. (C), Quantification of histone deacetylase (HDAC) activity in fibroblasts treated with DMSO or 10 μM blebbistatin for 2 hours (n=5). Significance determined by two-tailed, unpaired t test, compared to DMSO condition. (D), Quantification of H3K4-specific histone methyltransferase (HMT) activity in fibroblasts treated with DMSO or 10 μM blebbistatin for 2 hours (n=3). Significance determined by two-tailed, unpaired t test, compared to DMSO condition. (E), Quantification of H3K4-specific histone demethylase (HDM) activity in fibroblasts treated with DMSO or 10 μM blebbistatin for 2 hours (n=3). Significance determined by two-tailed, unpaired t test, compared to DMSO condition. (F), ATAC-seq tracks for Ascl1, Brn2, Myt1l and TUBB3 genomic loci from fibroblasts treated with DMSO or 10 μM blebb for 2 hours, highlighting promoter, proximal enhancer (Enhancer-P) and distal enhancer (Enhancer-D) sites. (G), ChIP-qPCR analysis shows the percent input increase of histone modifications at the promoter regions of Ascl1, Brn2, Myt1l and TUBB3 in BAM-transduced fibroblasts cultured with DMSO or 10 μM blebbistatin at day 3 (n=3). Significance determined by two-tailed, unpaired t test, compared to DMSO condition. (H), Representative images of 5-mC expression in non-transduced fibroblasts treated with blebbistatin for 24 hours. Scale bar, 20 μm. (I), Quantification of percentage of methylated DNA (5-mC) in total DNA from DNA samples of fibroblasts cultured in the absence and presence of blebbistatin for 24 hours (n=4). Significance determined by two-tailed, unpaired t test, compared to DMSO condition. (J), Quantification of DNA methyltransferase (DNMT) activity in fibroblasts treated with DMSO or 10 μM blebbistatin for 2 hours (n=5). Significance determined by two-tailed, unpaired t test, compared to DMSO condition. Bar graphs show mean standard deviation (**p<0.01, ***p<0.001, ****p<0.0001).

FIG. 6. Optimal level of focal adhesion kinase inhibition improves iN cell generation. (A), Reprogramming efficiency of BAM-transduced fibroblasts cultured in the absence and presence of various concentrations of the FAK inhibitor, PF573228 (denoted as PF in this figure) (n=3). Significance was determined by a one-way ANOVA and Dunnett's multiple comparison test. (B), Immunofluorescent images of TUBB3⁺ iN cells derived in the presence of 1 μM PF573228 (PF) displaying a typical neuronal morphology and co-expressing mature neuronal markers, NeuN, MAP2 and synapsin. Scale bar, 50 μm. (C), Representative traces of spontaneous changes in membrane potential in response to current injection from iN cells obtained in the absence and presence of 1 μM PF573228. The inhibitor was administered during the first 7 days of reprogramming. (D), Western blot analysis of FAK and mesenchymal marker expression in BAM-transduced fibroblasts that were treated with various concentrations of PF573228 for 2 days. (E), qRT-PCR analysis of neuronal gene expression at day 5 from non-transduced (NT) and BAM-transduced fibroblasts cultured in the absence and presence of 5 μM PF573228 for 4 days (n=3). Expression level normalized to BAM-transduced fibroblasts treated with DMSO. Significance determined by two-tailed, unpaired t test, compared to the DMSO condition for transduced cells for the same gene. Bar graphs show mean±standard deviation (*p<0.05, **p<0.01).

FIG. 7. Focal adhesion kinase inhibition modulates the epigenetic state to enhance iN reprogramming. (A), Representative images of histone modifications in non-transduced fibroblasts after treatment with 5 μM PF573228 for 2 hours. Scale bar, 10 μm. (B), Quantification of histone acetyltransferase (HAT) activity in fibroblasts treated with DMSO or 5 μM PF573228 for 2 hours (n=5). Significance determined by two-tailed, unpaired t test, compared to DMSO condition. (C), Quantification of histone deacetylase (HDAC) activity in fibroblasts treated with DMSO or 5 μM PF573228 for 2 hours (n=5). Significance determined by two-tailed, unpaired t test, compared to DMSO condition. (D), Quantification of H3K4-specific histone methyltransferase (HMT) activity in fibroblasts treated with DMSO or 5 μM PF573228 for 2 hours (n=3). Significance determined by two-tailed, unpaired t test, compared to DMSO condition. (E), Quantification of H3K4-specific histone demethylase (HDM) activity in fibroblasts treated with DMSO or 5 μM PF573228 for 2 hours (n=3). Significance determined by two-tailed, unpaired t test, compared to DMSO condition. (F), ATAC-seq tracks for Ascl1, Brn2, Myt1l and TUBB3 genomic loci from fibroblasts treated with DMSO or 5 μM PF for 2 hours, highlighting promoter, proximal enhancer (Enhancer-P) and distal enhancer (Enhancer-D) sites. G, ChIP-qPCR analysis shows the percent input increase of histone modifications at the promoter regions of Ascl1, Brn2, Myt1l and TUBB3 in BAM-transduced fibroblasts cultured with DMSO or 5 μM PF573228 at day 3 (n=3). Significance determined by two-tailed, unpaired t test, compared to DMSO condition. (H), Representative images of 5-mC expression in non-transduced fibroblasts treated with 5 μM PF573228 for 24 hours. Scale bar, 20 μm. (I), Quantification of percentage of methylated DNA (5-mC) in total DNA from DNA samples of fibroblasts cultured in the absence and presence of PF573228 for 24 hours (n=4). Significance determined by two-tailed, unpaired t test, compared to DMSO condition. (J), Quantification of DNA methyltransferase (DNMT) activity in fibroblasts treated with DMSO or 5 μM PF573228 for 2 hours (n=5). Significance determined by two-tailed, unpaired t test, compared to DMSO condition. Bar graphs show mean±standard deviation (***p<0.001, ****p<0.0001).

FIG. 8. Reduction in cell adhesions using biomaterials promotes iN reprogramming. (A), Immunofluorescent staining of actin network (phalloidin), phospho-FAK, and nuclei (DAPI) in non-transduced fibroblasts cultured in tissue culture-treated wells (TC), flat PDMS membranes (Flat), and PDMS membranes with 10-μm microgrooves. Scale bar, 50 μm. (B), Reprogramming efficiency of BAM-transduced fibroblasts cultured in tissue culture-treated wells (TC), flat PDMS membranes (Flat), and PDMS membranes with 10 μm grooves (n=4). Significance was determined by a one-way ANOVA and Tukey's multiple comparison test.

FIG. 9. iN Reprogramming of 3D Spheroids and 2D culture. (A) Schematic of reprogramming timeline. (B-C) At 2 days after Dox activation (1 day after spheroid formation), staining of 0-tubulin III (Tuj1) and nuclei (DAPI) in iN spheroids (B) and 2D culture (C). (D-E) At 2 weeks after reprogramming induction, Tuj1 staining of replated spheroids (D) and replated single cells (E). (F) Quantification of conversion efficiency after two weeks of reprogramming. *p<0.05 (n=3).

FIG. 10. Effects of TGF-β and BMP Pathway Inhibition on Spatial iN Reprogramming. After 3 days of reprogramming, staining of spheroids for Tuj1 (green) and DAPI (blue), including graphical depictions of their relative intensity over the diameter of the spheroid, for (A) controls, (B) application of both TGF-0 type I receptor ALK4/5/7 inhibitor, A83-01 (A), and BMP receptor kinase ALK2 inhibitor, K02288 (K), (C) A alone, and (D) K alone.

FIG. 11. Illustrative microfluidic cell culture system. The left panel shows a photograph of a lab on chip embodiment of the invention and the right panel shows an enlarged image of a microfluidic cell culture system comprising an inlet reservoir, an outlet reservoir and a single channel coupling the inlet reservoir to the outlet reservoir.

FIG. 12. Significance of microchannel cross-section and aspect ratios on cellular reprogramming efficiency. (A) diagram showing a microchannel cross-section and a cell cross section, and formula for determining channel aspect ratios and cellular cross section area ratios. (B) data from studies showing the effects of aspect ratios and cross section area ratios on cellular reprogramming efficiency in fibroblasts (left panel) and macrophages (right panel).

FIG. 13. Forced nuclear deformation increases the efficiency of gene silencing by CRISPR/Cas9. (A) Experimental timeline for sRNA/Cas9 transfection, nuclear deformation and analysis of CRISPR/Cas9-mediated genome silencing. (B) The cells were transfected with Chd1-sgRNA and a DNA construct for Cas9. 24 hours later, the cells were subjected to nuclear deformation by flowing through the microfluidics device. Then cells were cultured in DMEM+10% fetal bovine serum (FBS for 72 hours. Western blotting was used to detect the Chd1 protein level. Sqz: squeezed.

FIG. 14. Forced nuclear deformation promotes CRISPR/dCas9-mediated neuronal gene activation and induced neuronal (iN) efficiency. (A) Experimental timeline for the transfection of cells with sgRNA and dCas9-activation system, nuclear deformation, and iN reprogramming analysis. (B) Bulk ATAC-seq detect the chromatin accessibility 3 hours after cell pass through the microchannels. The representative ATAC-seq result shows the chromatin accessibility changes at the promoter and enhance of Ascl1 gene. Sqz: squeezed. (3) Based on ATAC-seq results, various Ascl1 sgRNAs were designed to target the sites with an increase of accessibility. Then gRNA/dCas9 gene activation system was transfected into the fibroblasts by Lipofectamine™ Stem Transfection Reagent (ThermoFisher). After 24 hours, cells were subjected to nuclear deformation by using microfluidics device. The cells were cultured in N2B27 neuronal indication medium for 14 days, fixed, and stained for neural cell marker Tuj1. The iN reprogramming efficiency was calculated as the percentage of Tuj1⁺ iN cells normalized to the number of cells plated at Day 1. the Cells without CRISPR/dCas9 transfection were used as Sham. Cells were not subjected to nuclear deformation were used as Control. Here ATAC-seq guided the design of 3 gRNAs, and the experiment further demonstrated that gRNA2 was the most efficient.

FIG. 15. Substrate stiffness-induced biphasic enhancement of iN reprogramming efficiency. (a) Experimental timeline for substrate stiffness-induced iN reprogramming. (b) Quantification of cell area based on phalloidin staining (n>50 cells). Fibroblasts were seeded onto polyacrylamide (PAAm) gels of varying stiffness or glass, which served as control in all experiments, for 24 hours. (c) Quantification of nuclear volume based on DAPI staining and confocal microscopy from fibroblasts cultured of PAAm gels or glass for 24 hours (n>30 cells). (d) The percentage of cells in the S-phase of the cell cycle based on propidium iodide staining. Fibroblasts were seeded on PAAm gels for 48 hours, collected and stained with propidium iodide followed by flow cytometry analysis (n===3). (e) Reprogramming efficiency of BAM-transduced fibroblasts that were cultured on glass and PAAm gels of varying stiffness (n=4). On day 7, the cells were fixed and stained for Tubb3 by Tuj1 antibody, followed by immunofluorescence microscopy to quantify Tuj1+iN cells. (f) Relative Ascl1 mRNA expression in DAM-transduced fibroblasts seeded on glass and PAAm gels of varying stiffness at day 1 following Dox activation (n=3). (g) Fibroblasts transduced with BAM and an Ascl1 promoter-GFP construct were seeded on glass and PAAm gels of varying stiffness for 24 hours and activated by Dox for another 24 hours. The cells were fixed and observed by immunofluorescence microscopy, showing that 20 kPa-PAAm gel induced more Ascl1 promoter-GFP-+ cells at day 1, as quantified in the bar graph (n=−3). (i) Representative images of Tuj1+ cells expressing mature neuronal markers, NeuN, MAP2 and synapsin at 21 days after cells were cultured on 20 kPa gels. Scale bar, 100 μm. (i) Representative trace showing spontaneous changes in membrane potential in response to current injection from iNs derived on 20 kPa gels. In b-g, statistical significance was determined by a one-way ANOVA and Tukey's multiple comparison test (N.S.: Not significant, *p<0.05, ***p<0.001). In b-c, box plots show the ends at the quartiles, the mean as a horizontal line in the box, and the whiskers represent the standard deviation (SD). In d-g, bar graphs show mean±SD.

FIG. 16. Matrix stiffness modulates histone acetyltransferase (HAT) activity and histone acetylation. (a) Immunofluorescent images of AcH3 in non-transduced fibroblasts cultured on glass and PAAm gels of various stiffness for 2 days. Scale bar, 10 μm. (b) Quantification of AcH3 intensity in cells cultured on glass and PAAm gels of various stiffness (based on experiments in a) (n>40). (c, d) ChIP-qPCR analysis shows the percent input of AcH3 at the promoter regions of Ascl1 (c) and Tubb3 (d) in BAM-transduced fibroblasts cultured on glass and PAAm gels of varying stiffness at day 3 (n=3). (e) Volcano plot showing differential accessible regions. Red dots indicate regions with increased chromatin accessibility and blue dots regions with decreased accessibility. (f) Heatmap representation of differentially accessible regions that overlap with Ascl1 ChIP-seq peaks (GSE43916: SRX323557). Each row represents a differential region; each column is one biological replicate of the indicated condition. (g) Quantification of HAT activity in fibroblasts cultured on glass and PAAm gels of various stiffness for 2 days, as determined using the Epigenase HAT activity kit (n=6). (h) Quantification of HAT activity in fibroblasts cultured on glass and PA gels of various stiffness for 1 day followed by treatment with vehicle control (DMSO) or a HAT inhibitor (anacardic acid, AA) for 24 hours (n=4). (i) Reprogramming efficiency of BAM-transduced fibroblasts cultured on matrices of varying stiffness and pre-treated with a HAT inhibitor, anacardic acid, for 24 hours before adding Dox (n=4). In b-i, statistical significance was determined by a one-way ANOVA and Tukey's multiple comparison test (N.S.: Not significant., *p<0.05, **p<0.01, ***p<0.001). In b, box plots show the ends at the quartiles, the mean as a horizontal line in the box, and the whiskers represent the SD. In c-i, bar graphs show mean±SD.

FIG. 17. Material properties of hydrogels. (a) Elastic moduli of alginate hydrogels with different crosslinker concentrations. (b) Stress relaxation of covalently (elastic) and ionicaly crosslinked (viscoelastic) hydrogels. (c) Rheology measurements of representative covalenty and ionically crosslinked hydrogels. (d) Percentage of cells with Tuj1 expression on hydrogels with different mechanical properties on day 7. (e) Representative images of Tuj1+ cells on different hydrogels (scale bar, 100 μm). (f) HDAC activity of cells seeding on hydrogels with different mechanical properties. Lower HDAC activity indicates more histone acetylation (euchromatin mark).

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

Cell reprogramming has wide applications in tissue regeneration, disease modeling and personalized medicine, but low reprogramming efficiency remains a challenge. In addition to biochemical cues, biophysical factors can modulate the epigenetic state and a variety of cell functions. However, how biophysical factors help overcome the epigenetic barrier for cell reprogramming is not well understood.

The invention described herein involves using an innovative microfluidic device, specific cytoskeletal and adhesion inhibitors, and three-dimensional (3D) spheroids to significantly promote cell reprogramming, respectively, more specifically direct induced neuronal reprogramming. Cell reprogramming represents a major advancement in biology, and has wide applications in regenerative medicine, disease modeling and drug screening. Induced pluripotent stem cells (iPSCs) can be generated from somatic cells by the forced expression of Oct4, Sox2, KLF4 and c-Myc (OSKM) and/or other factors. Direct reprogramming is the process of converting from one cell type into a very distantly related cell type without proceeding through an intermediate pluripotent stage. For example, it has been shown that mouse embryonic and postnatal fibroblasts can be converted into induced neuronal (iN) cells via the forced expression of three transcription factors: Ascl1, Brn2 and Myt1l (BAM). This approach was later applied to generate iN cells from human fibroblasts using Ascl1, Brn2 and Myt1l and NeuroD1 (BAMN). This direct reprogramming approach has tremendous implications not only for basic science, but also for disease modeling and therapeutic testing, functionalizing knowledge for engineering improved replacement cells and tissues, and in situ regeneration and healing of cells already in or near the site of injury without the cancer risks of pluripotent cell products. Despite the potential of this technology, low and variable efficiencies in cell reprogramming, in particular, during iN conversion, are significant roadblocks to their application. Therefore, this invention sets to overcome this barrier by providing distinct methods to enhance direct iN reprogramming.

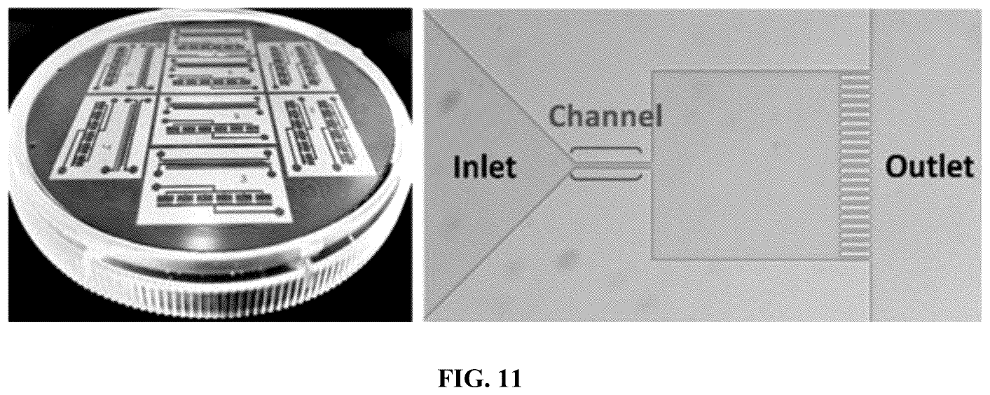

As discussed in detail below, the invention disclosed herein has a number of embodiments. Embodiments of the invention include microfluidic cell processing/culturing systems having elements including channels of a size/configuration selected to deform the nuclei of mammalian cells as they move/migrate through the channel. Embodiments of the invention include, for example, a microfluidic cell culture system comprising: an inlet reservoir configured to receive cells; an outlet reservoir to remove cells from the cell culture system; and at least one channel coupling the inlet reservoir to the outlet reservoir. In such microfluidic cell culture systems, the at least one channel is configured so that a mammalian cell contacts the sides and bottom of the channel and undergoes cellular and nuclear deformation as the cell migrates from the inlet reservoir through the channel to the outlet reservoir. In illustrative working embodiments of the invention disclosed herein, the inlet reservoir, the outlet reservoir and the at least one channel are disposed on a polydimethylsiloxane matrix in a lab on a chip configuration (see, e.g., FIG. 11). Certain embodiments of the invention further modulate cell adhesion by engineering 3D biomaterials (pore sizes, charge, hydrophilicity/hydrophobicity/zwitterionic property, etc. of the device as well as materials processed through the device) or 2D surfaces to increase chromatin accessibility for cell reprogramming and gene editing.

In typical embodiments of the invention, the microfluidic cell system of the invention further comprises mammalian cells. Typically, mammalian cells are selected for use in a microfluidic cell culture system embodiment of the invention by determining the size of the mammalian cell and further selecting a width and/or architecture of the at least one channel in the microfluidic cell culture system such that the mammalian cell contacts the channel so as to undergo cellular and/or nuclear deformation as the cell migrates from the inlet reservoir through the channel to the outlet reservoir. In certain embodiments of the invention, the mammalian cells are selected to be or configured as three-dimensional spheroids. In illustrative embodiments of the invention, the mammalian cells are somatic cells, induced pluripotent stem cells. In some embodiments of the invention, mammalian cells disposed within microfluidic cell culture system are genetically modified and/or combined with an agent selected to modulate the physiology of the mammalian cells such as a cytoskeleton inhibitor, an adhesion inhibitor (e.g. PF573228), a TGF-3/Activin pathway inhibitor (e.g. A83-01), a BMP pathway inhibitor (e.g. K02288), and blebbistatin. In illustrative embodiments of the invention, the agents are small molecule agents (i.e., molecules having a molecular weight of <900 Da). Such agents can be used to decrease actin cytoskeleton tension or cell adhesion to increase chromatin accessibility and thus enhance the efficiency of cell reprogramming and gene editing. In certain embodiments of the invention, the mammalian cells disposed in the microfluidic cell culture system are transduced with an exogenous nucleic acid (e.g., one encoding a mammalian transcription factor such as Ascl1, Brn2 or Myt1l).

In certain embodiments of the invention, the mammalian cells are modified by a CRISPR ribonucleoprotein complex or CRISPR process. As used herein, the phrase “CRISPR ribonucleoprotein complex” refers to a ribonucleoprotein complex having CRISPR-associated endonuclease activity. Exemplary CRISPR ribonucleoprotein complexes include CRISPR/Cas9 CRISPR-associated endonuclease activity and CRISPR/Cpf1 CRISPR-associated endonuclease activity. CRISPR/Cas9 gene targeting requires a custom single-lead RNA (sgRNA) consisting of a targeted sequence (crRNA sequence) and a Cas9 nucleic acid recruitment sequence (tracrRNA). The crRNA region is a sequence of about 20 nucleotides, homologous to one of the regions of the gene you are interested in, that will guide the activity of the Cas9 nuclease. Examples of these CRISPR ribonucleoprotein complexes, the CRISPR-associated RNA and protein components, and CRISPR-associated endonuclease systems are disclosed in the following references: Collingwood, M. A., Jacobi, A. M., Rettig, G. R., Schubert, M. S., and Behlke, M. A., “CRISPR-BASED COMPOSITIONS AND METHOD OF USE,” U.S. patent application Ser. No. 14/975,709, filed Dec. 18, 2015, published now as U.S. Patent Application Publication No. US2016/0177304A1 on Jun. 23, 2016 and issued as U.S. Pat. No. 9,840,702 on Dec. 12, 2017; and Behlke, M. A. et al. “CRISPR/CPF1 SYSTEMS AND METHODS,” U.S. patent application Ser. No. 15/821,736, filed Nov. 22, 2017, and U.S. Patent Application Publication No. 20190032131, the contents of which are hereby incorporated by reference herein in their entirety.

As discussed in the Examples below, we utilized selected microfluidic channel widths and/or architectures to induce a millisecond deformation of the cell nucleus, which caused the wrinkling and transient disassembly of the nuclear lamina, local detachment of lamina-associated domain in chromatin, and a decrease of histone methylation (H3K9me3) and DNA methylation. In this context, we have discovered that such mechanical squeezing results in global changes in chromatin which then enhances associated efforts to modulate the physiology of cells, for example the engineering and/or reprogramming of cells (e.g. reprogramming fibroblasts into neurons). This mechanopriming approach can enhance other reprogramming processes such as turning macrophages into neurons and converting fibroblasts into induced pluripotent stem cells. In addition, this mechanopriming approach can further be used to modulate other cell engineering methods.

In view of the discovery that mechanopriming approaches using selected cells and channel sizes in a microfluidic cell culture system can enhance cellular reprogramming as well as a number of other genetic engineering processes, embodiments of the invention include methods of culturing, reprogramming, and engineering cells in the microfluidic cell culture systems of the invention. For example, embodiments of the invention include a method of culturing a mammalian cell comprising selecting the mammalian cell for culturing in a microfluidic cell culture system of the invention, wherein selecting the mammalian cell comprises determining the size and/or cross-section area ratio of the mammalian cell and/or the mammalian cell nucleus, and further selecting a width and/or architecture (e.g. aspect ratio) of the at least one channel in the microfluidic cell culture system such that the mammalian cell contacts the channel so as to undergo cellular and/or nuclear deformation as the cell migrates from the inlet reservoir through the channel to the outlet reservoir, such that the cells are cultured in the microfluidic cell culture system. Typically in these methods, the mammalian cells are selected to be mammalian cells that have formed three dimensional aggregates.

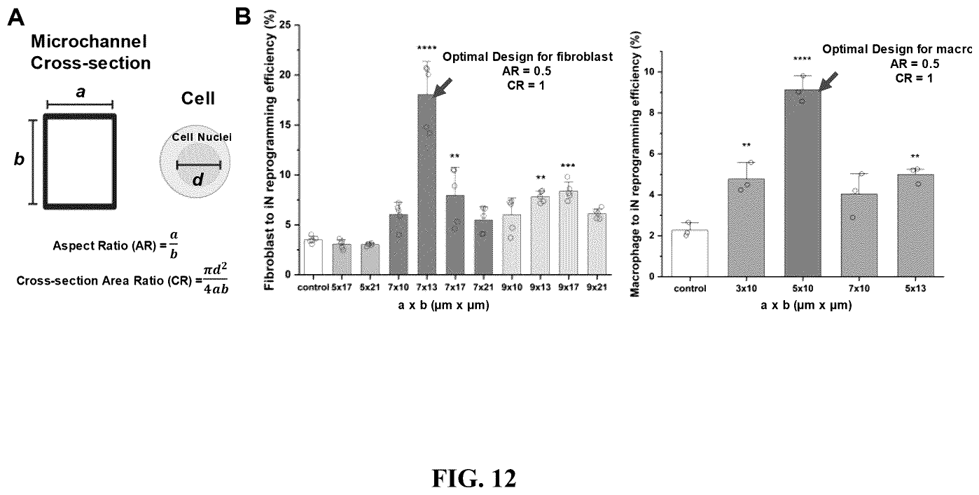

Studies have shown the capability of the microfluidics device to promote gene editing efficiency via cell nucleus deformation. As disclosed herein, we have discovered that the design of the cross-section geometry of microchannels of the invention is a critical factor of this enhancement of methods such as gene editing. In order to observe significant criterium for channel design and selection, we varied two dimensionless numbers: Aspect Ratio (AR)=Channel Width/Channel Height, and Cross-section Area Ratio (CR)=Cross-sectional Area of Cell Nuclei/Cross-sectional Area of Microchannel (FIG. 12A). As shown by the data presented in FIG. 12, we observed cell reprogramming in different channel widths and architectures where mouse fibroblasts were converted into neurons as a model to observe appropriate AR and CR parameters that result in the highest gene editing efficiency. We performed these cell reprogramming experiments using two different cell types, mouse ear-derived fibroblasts (MERF) with an averaged cell nucleus diameter of 11 μm, and mouse macrophages with an averaged cell nucleus diameter of 8 μm. The reprogramming efficiency to convert these two cell types into induced neuronal (iN) cells was used as a readout to identify appropriate AR and CR parameters.

As noted above, in certain methodological embodiments of the invention, the average diameter of a cell and/or the average diameter of the cell nucleus and/or the cross-section area ratio of the cell or cell nucleus is determined (e.g. the average cellular or nuclear diameter a particular cell type such as a fibroblast, a T cell, a macrophage etc.) and this information is then used to select the size and/or architecture of the microfluidic channel to be used with these cells. Typically in such methods of the invention, the diameter of the channel is selected to be smaller than the diameter of the cell and/or the cell nucleus, and not larger than the diameter of the cell. In certain embodiments of the invention, the at least one channel is selected to be at least 2 μm and not more than 200 m in width (e.g., a channel less than 3 μm, 7 μm, 10 μm, 15 μm, 20 μm or 25 μm, 50 μm or 100 μm in width). In some embodiments of the invention, the aspect ratio of the channel is further designed or selected. For example, certain embodiments of the invention selectively utilize channels having an aspect ratio between 0.2 and 1 such as an AR from 0.25 to 0.75 (e.g., an aspect ratio of about 0.5, such as an aspect ratio within 10% of 0.5). In addition, certain embodiments of the invention selectively utilize cells observed to exhibit a cross-section area ratio between 0.75 and 1.25 (e.g., a cross-section area ratio of about 1, such as a cross-section area ratio within 10% of 1). While the channels typically exhibit a rectangular cross-sectional architecture, other shapes (oval, polygonal) of microchannel cross-section can work. In general, if the dimension of microchannels in one direction is smaller than cell nucleus size, this is observed improve gene editing efficiency and cell reprogramming.

For the experiments shown in FIG. 12, cells were transduced with lentiviral constructs to express reprogramming factors Ascl1, Brn2 and Myt1l for the induction of neuronal genes. Then the cells were migrated through microchannels with various cross-section areas (different AR and CR), including 10 different sizes of the microchannel for MERF and 4 sizes of microchannels for macrophages. As shown in FIG. 12B, a microchannel with a width of 7 μm and a height of 13 μm was optimal for fibroblasts and induced the highest level of reprogramming efficiency, and a channel with a width of 5 μm and a height of 10 μm was optimal for macrophages (FIG. 12B). These results suggest that the optimal cross-section would have AR˜0.5 and CR˜1. Since the distribution of cell nucleus sizes may vary for each cell type, which may affect the average nucleus diameter, the optimal levels may fluctuate around these AR and CR values. Based on our findings, in typical embodiments of the invention, (1) either the width or height of the microchannel cross-section should be less than the nucleus diameter to effectively induce changes in chromatin organization and improve cell reprogramming; (2) the optimal cross-section would have AR-0.5 and CR-1; (3) an AR between 0.2 and 1 may cause chromatin changes and improve cell reprogramming, if there is no significant effect on cell viability; (4) regarding CR, microchannels with CR<1 may exert squeezing to nucleus; if CR>1, either width or height of the microchannel cross-section needs to be <nucleus diameter.

This optimization process provides guidance for the methods of making and using the microfluidics devices of the invention disclosed herein. For example, in certain embodiments of the invention, the design of the microchannel is determined based on the cell nucleus size. In this context, optimization experiments with different cells and/or techniques can be run in initial tests to maximize gene editing efficiency for different types of cells, methods which can expand the applications of this innovative microfluidics device to different gene editing models. This can further expand the applications of this device in disease modeling, drug screening and cell therapy with different cell types. Moreover, personalized medicine will be possible by designing the microfluidics device individually based on the nucleus size of the cells collected from the patient.

In embodiments of the methods invention, selected cells are manipulated under such culture conditions where the cells are disposed in a microfluidic cell culture system. In some embodiments of the invention, a microfluidic cell culture system of the invention is used to facilitate reprogramming and/or differentiation of mammalian cells. In some embodiments of the invention, a microfluidic cell culture system of the invention is used to boost the efficiency of gene editing and the like of mammalian cells, In one such embodiment, somatic cells are induced to reprogram into pluripotent stem cells in this microfluidic cell culture system. In certain embodiments, somatic cells are induced to reprogram into neuronal cells in this microfluidic cell culture system. Typically in these methods, the mammalian cells are further combined with an agent selected to modulate the physiology of the mammalian cells such as a cytoskeleton inhibitor, an adhesion inhibitor (e.g. PF573228), a TGF-β/Activin pathway inhibitor (e.g. A83-01), a BMP pathway inhibitor (e.g. K02288), and blebbistatin. Optionally in these methods, the mammalian cells comprise an exogenous nucleic acid (e.g., an expression vector such as one including an inducible promoter). In certain methods of the invention, the size of the at least one channel in the microfluidic cell culture system is selected such that the mammalian cell contacts the channel and experiences transient disassembly of nuclear lamina as the cell migrates from the inlet reservoir through the channel to the outlet reservoir. Typically in these methods, the cells undergo nuclear deformation for less than one second.

A related embodiment of the invention is a method of culturing (and typically modulating the physiology) of mammalian cells comprising disposing mammalian cells in the inlet reservoir of a microfluidic cell culture system disclosed herein, and then migrating the mammalian cells from the inlet reservoir through the at least one channel to the outlet reservoir; wherein the mammalian cells undergo mechanical deformation as the cells migrate from the inlet reservoir through the channel to the outlet reservoir (i.e, mammalian cells cultured in the microfluidic cell culture system are mechanical squeezed as they migrate through channels/conduits in the microfluidic cell culture system).

As discussed in the Examples below, we utilized microfluidic channels of selected widths to induce a millisecond deformation of the cell nucleus, which caused the wrinkling and transient disassembly of the nuclear lamina, local detachment of lamina-associated domain in chromatin, and a decrease of histone methylation (H3K9me3) and DNA methylation. As disclosed herein, we further discovered that such global changes in chromatin at the early stage of cell reprogramming boosted the conversion of fibroblasts into neurons. Consistently, inhibition of H3K9 and DNA methylation partially mimicked the effects of mechanical squeezing on iN reprogramming efficiency, while the inhibition of H3K9 demethylase blocked nuclear deformation-enhanced reprogramming. In addition, knocking down lamin A had similar effects to mechanical squeezing. Furthermore, this mechanopriming approach can enhance other reprogramming processes such as turning macrophages into neurons and converting fibroblasts into induced pluripotent stem cells, and can be scaled up to mechanically modulate epigenetic state for cell engineering.

Embodiments of the invention include a polydimethylsiloxane (PDMS) based microfluidics lab-on-chip as shown in FIG. 11, one where the template includes three different channel widths (3 μm, 5 μm and 7 μm) and 10 μm height, with each device containing 20 parallel aligned channels. In illustrative methods for using such devices, mouse ear-derived fibroblasts are infected with Doxycycline (Dox)-inducible lentiviral vectors for BAM for 24 hours, then the media is replaced to 10% FBS DMEM medium for 12 hours, followed by the addition of Dox for 6 hours. Upon passaging the cells using trypsin and re-suspending in DMEM medium without FBS, 1*10⁶ cells/ml pass through the channel at a flow velocity rate of 20 μl/min. After cells pass through the device, cells are cultured on glass slides coated with 10 μmg/ml fibronectin for 3 hours, enabling the cells to attach and for the cell membrane to recover. The media is then changed to N2B27 medium for neural induction.

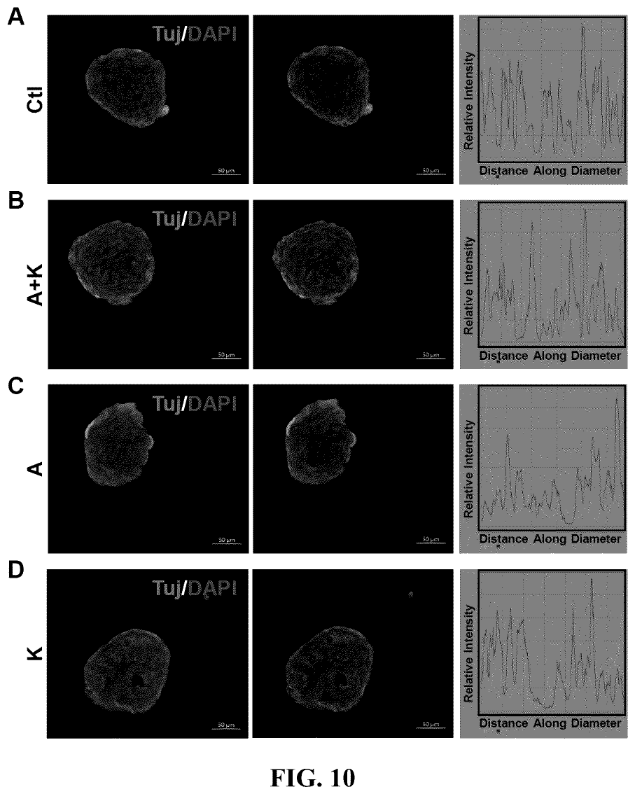

Moreover, we have discovered that several small molecule compounds that disrupt intracellular structures can be utilized to enhance iN reprogramming. In illustrative reprogramming experiments, adult mouse ear fibroblasts were transduced with doxycycline (Dox)-inducible lentiviral vectors encoding for BAM, and seeded onto laminin coated tissue culture dishes. Twenty-four hours after plating, Dox was added into media composed of DMEM, 10% FBS and 1% Penicillin/Streptomycin. The following day the media was replaced to N2B27 medium wherein fibroblasts were treated with various chemical inhibitors for first 7 days. Two weeks after post-Dox induction, iN cells were identified via immunostaining for neuronal beta-III tubulin (TUJ1) and the reprogramming efficiency was determined. We have found that disruption of actin-myosin contractility via treatment with blebbistatin enhanced the efficiency of iN conversion (i.e. 4.5-fold increase compared to control). Similarly, inhibition of cell adhesion using the focal adhesion kinase inhibitor, PF573228, also promoted iN reprogramming in a biphasic manner (FIG. 10C). Furthermore, our findings suggest that the disruption of focal adhesions and cytoskeletal contractility modulates the epigenetic state of the chromatin and the expression of neuronal genes.

In addition, we have also discovered that the use of cells selected to be in 3D spheroids in the microfluidic systems of the invention not only promotes earlier onset of iN reprogramming, but unexpectedly enhances reprogramming efficiency by over 67-fold after just 2 weeks, relative to conventional two dimensional (2D) methods. The enhancement displays a characteristic spatial distribution of a peripheral layer enriched with neurons surrounding an unreprogrammed spheroid core. Inhibition of the TGF-β/Activin and BMP pathways, which suppresses the mesenchymal phenotype, helps remove the spatial heterogeneity of reprogramming for improved efficiency. This combination of microfluidic cell systems, 3D spheroid cultures and chemical inhibitors in iN generation is a powerful technology to improve the translation of iN conversion.

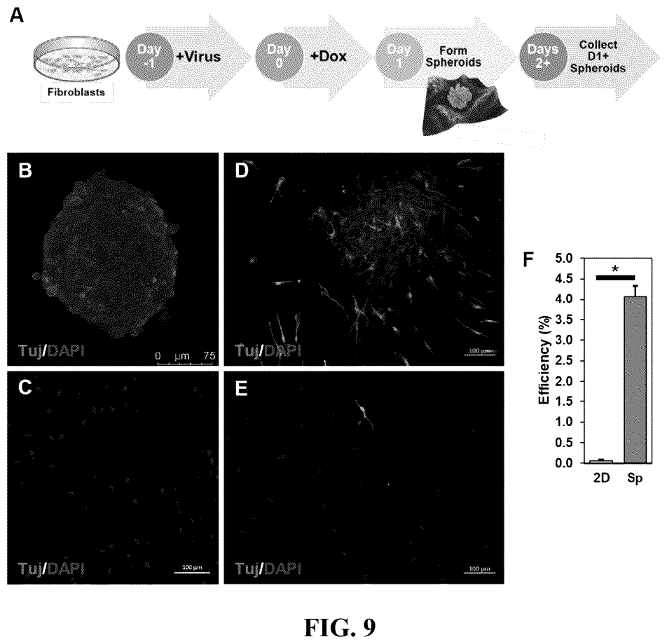

For the reduction to practice, primary human neonatal dermal fibroblasts (hNDFs) were transduced with doxycycline (Dox)-inducible lentiviral vectors for the BAMN factors. After Dox induction in monolayer to ensure unbiased activation of the transgenes, hNDFs were either plated onto Matrigel-coated cover slips as 2D controls or centrifuged in microwells to form 3D aggregates, or “spheroids” (FIG. 9A). The expression of neuron-specific β-tubulin III (Tuj1) was used as a marker of neuronal fate, as is convention. Tuj1 expression began earlier in spheroids than in 2D culture, appearing as early as day 2 (two days after Dox induction and one day after spheroid formation) (FIG. 9BC). To evaluate relative reprogramming efficiency, spheroids were replated onto Matrigel-coated cover slips after three days without enzymatic disaggregation. At two weeks post-dox induction, monolayer iNs still displayed very few Tuj1+ cells (0.06%; FIG. 9E). Spheroid iNs, in contrast, had dramatically improved neural conversion (4.06%, FIG. 9D), by over 67-fold (FIG. 9F).

Inhibition of the TGF-β/Activin/Nodal pathway with the TGF-β type I receptor ALK4/5/7 inhibitor, A83-01 (A) (FIG. 10C), or the BMP pathway with BMP7 type I receptor kinase ALK2 inhibitor, K02288 (K) (FIG. 10D), alone did not affect spatial Tuj1+ reprogramming patterns relative to no-treatment controls (FIG. 10A). Application of both chemicals for dual pathway inhibition, however, enhanced Tuj1 expression on the spheroid interior without eliminating peripheral expression (FIG. 10B). These results suggest that the mesenchymal identity in the core of spheroids may be the barrier to neuronal reprogramming, and that targeting this barrier would provide even better conversion efficiency.

There are several innovative conceptual and technical aspects in this invention: (1) this microfluidics lab-on-chip is a high-throughput device which can process one million cells in one hour while retaining the viability of the majority of cells. More importantly, as the cells pass through the device, they undergo cell and nuclear deformation that unexpectedly leads to a 7-fold increase in the reprogramming efficiency compared to the control group. (2) Utilizing this biophysical cue-based chip to promote the reprogramming efficiency is a fast and minimally invasive approach, whereby the deformation experienced by cells in a short period of time (i.e. few seconds) is sufficient enough to significantly increase the reprogramming efficiency. (3) The proposed small molecules offer a simple and more efficient method to promote iN reprogramming from adult fibroblasts, compared to standard culture conditions, through the simple addition of these chemical compounds to the culture medium. (4) The 3D niche of spheroids has marked advantages in being easily scalable, as well as the capability of synergizing with biochemical factors (e.g. chemical inhibitors) to enhance conversion efficiency even further.

Further aspects and embodiments of the invention are discussed in the examples below.

### EXAMPLES

### Example 1: Transient Nuclear Deformation Primes Epigenetic State and Promotes Cell Reprogramming

Certain disclosure discussed in this Example is found in Song et al., Nature Materials volume 21, pages 1191-1199 (2022), the contents of which are incorporated by reference (hereinafter “Song et al.”).

Cell reprogramming technologies, such as somatic cell nuclear transfer, induced pluripotent stem cell (iPSC) reprogramming, and direct reprogramming, can be used to derive desirable cell types and have wide applications in regenerative medicine, disease modeling and drug screening¹⁻³. Direct reprogramming enables the conversion of one cell type into another desired cell type by circumventing the pluripotent stage and time-consuming differentiation process, as exemplified in the conversion of fibroblasts into neurons⁴, cardiomyocytes⁵, β-islet cells⁶, blood cell progenitors⁷, and hepatocytes⁸. However, the low efficiency of these conversion processes presents a barrier for biomedical applications. For example, mouse neonatal fibroblasts can be converted into induced neuronal (iN) cells via ectopic expression of three transcription factors: Ascl1, Brn2 and Myt1l (BAM), with an efficiency of 1.8-7.7%, but with a much lower efficiency for adult fibroblasts⁹.

A critical step in cell reprogramming is to overcome the epigenetic barrier of heterochromatin and turn on the endogenous genes for cell type conversion. Most of the previous studies have focused on the roles of transcriptional factors and biochemical factors in cell reprogramming^{10,11}, but the effects of biophysical factors are much less understood. Cells experience mechanical stimuli at both short and long time scales, from seconds to days, which may result in mechano-chemical signaling, cytoskeleton reorganization and chromatin changes¹²⁻¹⁷. For example, surface topography induces an elongated nuclear shape and increases histone H3 acetylation (AcH3) and H3K4 methylation during fibroblast reprogramming into iPSCs¹⁸, and three-dimensional (3D) collagen gel increases H3K4 methylation in T cells¹⁹. In addition, soft matrix decreases H3K9me3 in tumor cells in a cell type-dependent manner²⁰, and persistent uniaxial stretching of adhesive substrates decreases H3K9me3 in epidermal cells²¹. Interestingly, compression on the side of adherent mesenchymal cells enhances histone acetylation, while compression on the top of adherent fibroblasts leads to an increase of heterochromatin^{22,23}. These differential responses to various biophysical cues suggest that mechanotransduction to the nucleus is context-dependent in adherent cells, which may be attributed to the differences in specific biophysical cues, cell types, cell adhesions and cytoskeleton organization. Regardless of all these variations, we postulated that an appropriate mechanical perturbation of the cell nucleus could induce chromatin remodeling and help overcome the heterochromatin barrier for cell reprogramming. Cells in suspension offer a valuable model to test this hypothesis, in which the complexity of extracellular signals (e.g., adhesion polarity, matrix stiffness, ligand presentation) and intracellular components (focal adhesion complex, cytoskeleton organization) are removed or reduced. Therefore, to directly determine the effect of nuclear deformation on chromatin remodeling, we investigated whether mechanically squeezing suspended cells could regulate the epigenetic state and cell reprogramming, and explored the translation of the findings into mechano-biotechnology applications.

### Results

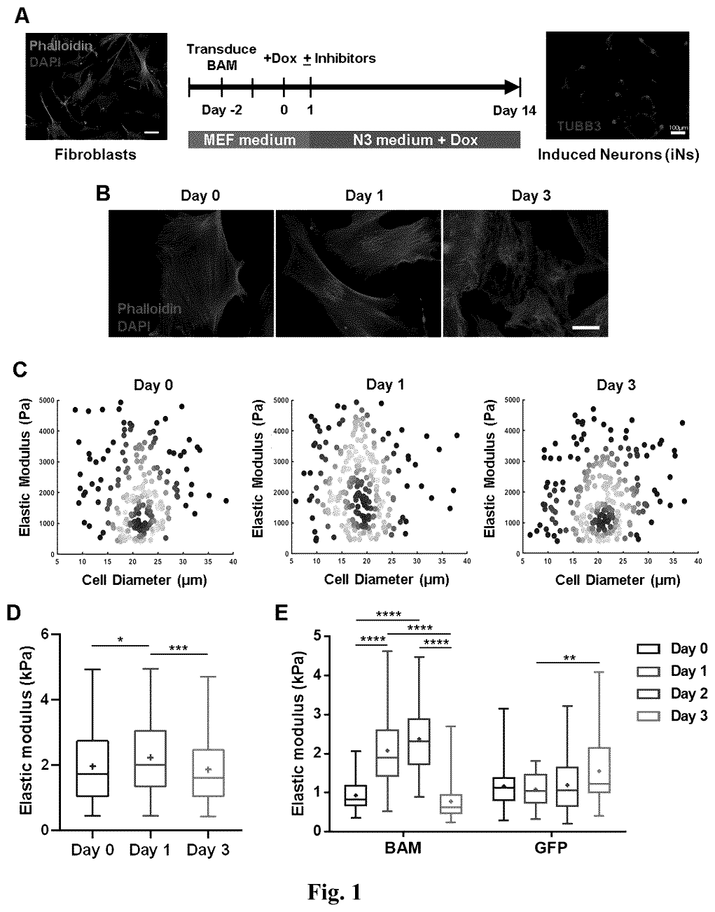

Microfluidic devices have been used to study how cell deformation affects gene transfer and cancer cell responses²⁴⁻²⁶, but smaller microchannels are needed to directly deform cell nucleus. To investigate the effect of nuclear deformation on direct reprogramming, we developed a microfluidic device with various sizes of constriction microchannels, and forced cells in suspension to flow through these channels (FIG. 1a and Fig. S1 in Song et al.). To determine a proper channel dimension that would enable nuclear deformation while maintaining cell viability, we measured the size of mouse fibroblasts (19.7±4.5 μm) and their nuclei (10.5±1.2 μm) (FIG. 1b in Song et al.), and fabricated microdevices that had parallel constriction channels with the same height (15 m) but different widths (3, 5, 7 and 9 μm). Cells passing through wide microfluidic channels (200 μm) did not show significant nuclear deformation (Fig. S2 in Song et al.), and were used as a control in all studies.

Cells were collected at the outlet of the microdevices and seeded onto fibronectin-coated wells. The cells passing through bigger channels (200, 9 and 7 μm wide) had negligible unattached cells after 3 hours, but 5-μm and 3-μm wide microchannels caused significant cell damage, where 19% and 35% of cells could not attach respectively (FIG. 1c in Song et al.). To determine the effect of nuclear deformation on cell membrane integrity, a fluorescently-tagged antibody (Cy5-Ab) was added to the culture media of fibroblasts in suspension before and during the introduction into the microfluidic device. In attached cells, Cy5-Ab could be detected in 31% and 76% of cells passing through 5-μm and 3-μm wide channels, respectively, suggesting that a transient cell membrane leakage occurred^{25,27} (FIG. 1d-e in Song et al.). This cell membrane leakage was also detected in 6% of cells passing through 7-μm wide microchannels but not in 9-μm or 200-μm wide channels. Additionally, Cy5-Ab was detected in 6.5% and 23% of the nuclei in the cells passing 5-μm and 3-μm wide microchannels but not bigger microchannels, indicating that microchannels less than 5-μm induced an increase of nuclear transport or nuclear membrane leakage (FIG. 1d-e in Song et al.). Furthermore, by using fibroblasts expressing histone 2B (H2B)-GFP, we observed that 3-μm and 5-μm wide channels, but not 7-μm and 9-μm wide channels, induced significant nuclear blebbing or segregation after squeezing (FIG. 1d and Fig. S3 in Song et al.). Consistently, in fibroblasts expressing an NLS-GFP (green fluorescent protein with a nuclear localization signal) reporter, there was a diffusion of NLS-GFP into the cytoplasm in >5% cells passing through 5-μm and 3-μm microchannels, indicating that nuclear rupture had occurred (Fig. S4 in Song et al.).

The rupture of cell membrane and nuclear envelope may induce DNA damage and cell death^{28,29}. We first performed live/dead cell staining and PrestoBlue assays to directly assess how microchannel width affected cell viability. In comparison to the control group (passing through 200-μm channel), 7 and 9 μm-wide channels did not induce noticeable cell death at 3 hours and one day after mechanical deformation (FIG. 1f and Fig. S5 in Song et al.). In contrast, after passage through 3 μm- and 5-μm wide channels, cell viability decreased significantly. In addition, in comparison to 7-μm and 9-μm wide channels that induced little DNA damage (2.2% and 0.7% respectively), 3-μm and 5-μm wide channels induced significant (20-50%) DNA damage (Fig. S6 in Song et al.). Based on these findings, we used 7-μm microchannels for the rest of the studies.

As flow rate could affect the rate of nuclear deformation and cell aggregation in microchannels, we examined cell viability and channel clogging at various flow rates in 7-μm microchannels. As shown in Fig. S7 in Song et al., the cell viability was not affected at a flow rate of 10 or 20 μL/minute. However, flow rates higher than 40 μL/min significantly decreased cell viability and caused cell aggregation and clogging in microchannels. Therefore, we used 20 μL/minute as an optimal flow rate to maintain a high throughput and cell viability. As measured by ultrafast imaging of the cell squeezing process, it took an average of 6.8 milliseconds (ms) for a cell to pass through a 7 μm-wide microchannel at 20 μL/minute flow rate (FIG. 1g in Song et al.), which resulted in a transient nuclear deformation.

To evaluate the effects of nuclear deformation on the mechanical properties of the nucleus, atomic force microscopy (AFM) was performed to measure the elastic modulus of cells at multiple time points after deformation. As shown in FIG. 1h in Song et al., in comparison to control cells, the stiffness of the cells, measured by indenting the plasma membrane of the cells above the nucleus, decreased significantly after cells were squeezed through 7-μm microchannels. After 24 hours, we observed a gradual recovery of elastic modulus, indicating that the mechanical changes induced by a millisecond squeezing lasted for hours. Additionally, we examined nuclear shape change by DAPI staining, and found that the cell nucleus was more elongated after squeezing, which gradually recovered after 24 hours (Fig. S8 in Song et al.).

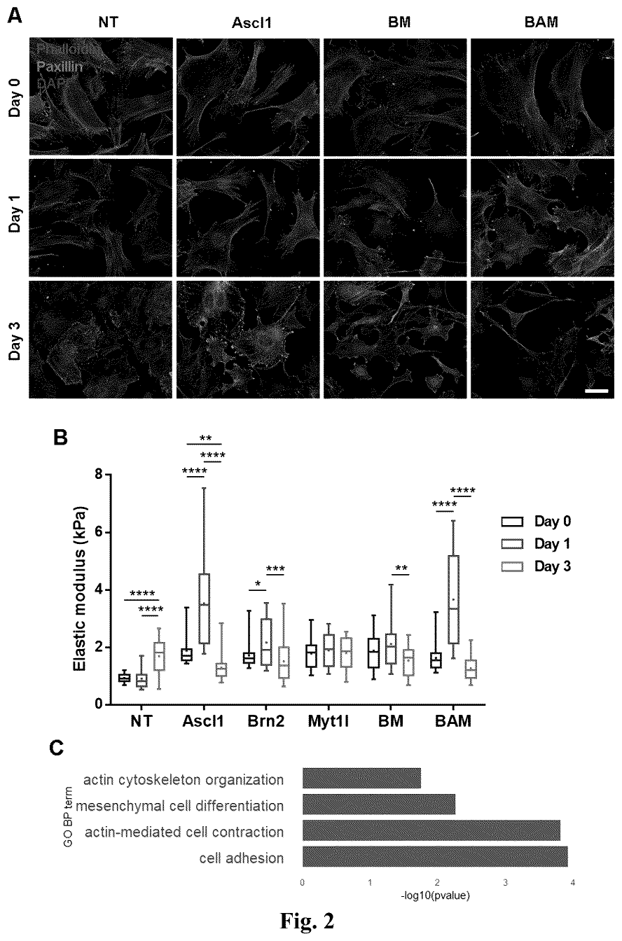

To determine whether microchannel-induced nuclear deformation had any effect on the direct conversion of fibroblasts into iN cells, adult mouse fibroblasts were transduced with doxycycline (Dox)-inducible lentiviral constructs containing the three reprogramming factors BAM as depicted in the timeline for the reprogramming experimental procedure (FIG. 2a in Song et al.). Two days later, Dox was added (designated as Day 0) to induce the expression of BAM transgenes and proteins within a few hours. To determine the timing of mechanical squeezing, before or after administering Dox, cells were introduced into microfluidic devices with 7-μm wide channels. Then the cells were collected and seeded onto fibronectin-coated glass coverslips and cultured in serum-free N2B27 medium (FIG. 2a in Song et al.). Seven days after induced mechanical deformation, cultures were fixed and stained for p-III tubulin (Tubb3) to determine the reprogramming efficiency. The reprogramming efficiency of cells going through 200-μm wide channels (as the control group) was not significantly different from the cells under static culture condition (Fig. S9 in Song et al.). Administering Dox at least 6 hours before subjecting the cells to nuclear deformation produced the highest reprogramming efficiency (˜8-fold) compared with the cells passing through 200-μm channels (FIG. 2b in Song et al.). These results suggested that the presence of BAM proteins within the first few hours after squeezing was critical and that a transient nuclear deformation was sufficient to enhance the reprogramming efficiency of fibroblasts into iN cells. In consistent with the effects of microchannel size on cell viability (FIG. 1f and Fig. S5 in Song et al.), microchannels smaller than 7-μm (i.e., 5 and 3-μm) decreased cell viability and thus compromised reprogramming efficiency, while 9-μm microchannels might not induce sufficient nuclear deformation and resulted in a lower reprogramming efficiency than 7-μm microchannels (FIG. 2c in Song et al.).

To determine whether nuclear deformation by microchannels facilitated the activation of endogenous neuronal genes, we monitored the effect of nuclear deformation on neuronal marker expression by quantitative polymerase chain reaction (qPCR) and live cell imaging. Among three transgenes, Ascl1 is a pioneer factor^{30,31}, so we examined how mechanical squeezing affected Ascl1 transgene expression and the activation of endogenous Ascl1. While endogenous Ascl1 expression in the control group showed a very low basal level at 6 hours and a 2-fold increase at 12 hours, squeezing cells triggered 4.6-fold to 15.4-fold induction of endogenous Ascl1 expression between 6-12 hours and a 10.6-fold increase at 24 hours when compared with the control group (FIG. 2d in Song et al.). Furthermore, to directly monitor the temporal activation of endogenous Ascl1 protein, fibroblasts were transduced with an Ascl1 promoter driven-GFP (Ascl1-GFP) and subjected to reprogramming process. Consistently, we found a significant increase in the number of Ascl1-GFP⁺ cells at day 1 in the squeezed cells compared to the control (FIG. 2e in Song et al.). On the other hand, the transgene expression of Ascl1 showed a slight increase (<2 fold) within 24 hours after squeezing (Fig. S10a in Song et al.), and the overall gene expression of Brn2 and Myt1l had less than 2.5-fold increase (Fig. S10b-c in Song et al.).

We also monitored the expression of other neuronal markers such as Tubb3. We first used gold nanorod biosensors³² with complementary sequence to detect mRNA expression of Tubb3 in living cells. Tubb3 mRNA expression was detectable as early as 12 hours in squeezed cells but rarely in the control group (FIG. 2f and Fig. S11 in Song et al.). Consistently, qPCR analysis showed significantly higher Tubb3 expression in the squeezed cells than the control group after 12 hours following nuclear deformation (FIG. 2g in Song et al.). In addition, we performed reprogramming experiments by using fibroblasts isolated from transgenic mice expressing a EGFP reporter driven by the promoter of neuronal gene Tau. The number of Tau-GFP⁺ cells in the squeezed group at day 4 was ˜6 times greater than that in the control group (FIG. 2h in Song et al.). We then used Tuj1 staining (for Tubb3) and neuron morphology analysis to determine reprogramming efficiency at various time points. At 1 week and 2 weeks after nuclear deformation, the iN reprogramming efficiency was significantly higher in the mechanically squeezed group compared with the control (FIG. 2i and Fig. S12 in Song et al.). Further characterization of the derived cells revealed that iN cells expressed mature neuronal markers microtubule associated protein 2 (MAP2) and Synapsin at 4 weeks after nuclear deformation (FIG. 2j in Song et al.). The terminally differentiated neurons from the control and squeezed groups had similar morphology (Fig. S13 in Song et al.). After 6 weeks, iN cells resulting from mechanical squeezing treatment showed calcium fluctuations, indicating a mature neuronal phenotype (Supplementary movie S1 in Song et al.)³³.

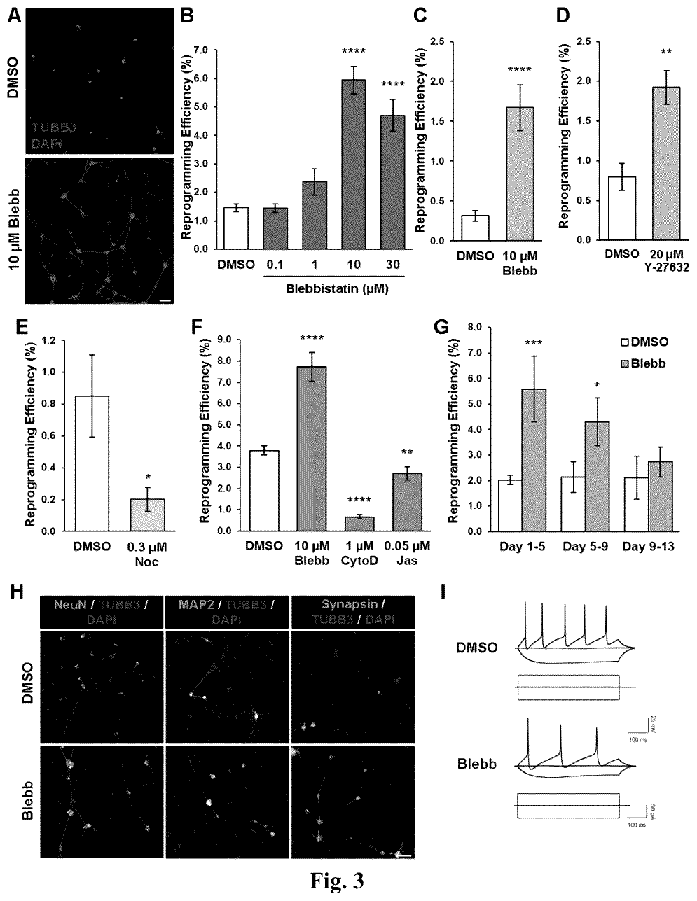

While nuclear deformation resulted in a modest transgene expression, which could also be achieved by increasing the titer of viral constructs, it could not fully account for the 8-fold induction of reprogramming efficiency. Therefore, we investigated the potential involvement of epigenetic changes that controls the on/off state of phenotypic genes. To investigate whether nuclear deformation-enhanced reprogramming efficiency was due to the changes in chromatin and epigenetic state, we first utilized a fluorescence resonance energy transfer (FRET) biosensor targeted at the nucleosome to monitor the levels of a heterochromatin mark H3K9me3. We found that H3K9me3 FRET signal significantly decreased in fibroblasts passing through 7-μm microchannels (FIG. 3a in Song et al.). To observe the temporal change in the same cells, we slowed down the squeezing process by lowering the pressure and flow rate in the microdevice. Cells passing through 200-μm channels did not show significant change in H3K9me3 FRET signal (Fig. S14 in Song et al.), whereas 7-μm microchannels decreased H3K9me3 FRET signal within 1 minute (Fig. S15 in Song et al.).

To determine whether the epigenetic changes persisted after squeezing and whether squeezing induced changes in other epigenetic marks, we performed immunostaining analysis of heterochromatin and euchromatin marks at multiple time points within the first 24 hours after cells passed through the microchannels. Consistently, we observed a significant decrease in H3K9me3 at 3 hours and 12 hours after nuclear deformation, which returned to the same level as the cells in the control group after 24 hours (FIG. 3b-c in Song et al.). Western blotting analysis also confirmed the global decrease of H3K9me3 induced by nuclear deformation (Fig. S16 in Song et al.). These results indicated that nuclear deformation resulted in a transient reduction of heterochromatin. In contrast, the levels of acetylated histone marks including AcH3 and H3K9ac, and histone methylation marks including H3K4me1, H4K20me3 and H3K27me3 did not show significant global changes in response to forced nuclear deformation (Fig. S17 in Song et al.). Western blotting analysis further confirmed that heterochromatin mark H3K27me3 did not show significant changes after squeezing, although H3K27me3 was inhibited by its inhibitor EED226 (Fig. S18 in Song et al.).

In addition to histone modifications, DNA methylation influences chromatin organization, which is critical for cell reprogramming³⁴. To investigate the effect of nuclear deformation on DNA methylation, we analyzed DNA condensation and the level of 5-methylcytosine (5-mC), a DNA methylation marker, in fibroblasts squeezed by microchannels. As shown in FIG. 3d-f in Song et al., both immunostaining analysis and enzyme-linked immunoassay (ELISA) (Fig. S19 in Song et al. shows a standard curve) showed that nuclear deformation significantly decreased DNA methylation for at least 12 hours. These results indicated that nuclear deformation caused chromatin modifications in both H3K9me3 and 5-mc.

To test whether the decrease in H3K9me3 played a role in iN conversion, BAM-transduced fibroblasts were treated with a H3K9-specific histone methyltransferase (HMT) inhibitor (Bix01294) for 24 hours. The inhibitor specifically suppressed H3K9me3 in a dose-dependent manner, and we selected a concentration of Bix01294 (1 μM) that did not affect cell viability (Fig. S20-22 in Song et al.). As shown in FIG. 3g in Song et al., Bix01294 partially mimicked the squeezing effect on the reprogramming efficiency. Additionally, pre-treatment with Bix01294, together with mechanical deformation, slightly increased the reprogramming efficiency compared to the squeezed-only group (FIG. 3g in Song et al.), suggesting that a 24-hour HMT inhibition by Bix01294 only led to a marginal enhancement. Furthermore, to determine whether the decrease of H3K9me3 was required for nuclear deformation-induced iN reprogramming, BAM-transduced fibroblasts were pre-treated with JIB-04 (100 nM), a H3K9 specific histone demethylase (HDMT) inhibitor, for 24 hours before being introduced into the microdevice. JIB-04, which significantly increased H3K9me3 (Fig. S20 in Song et al.), not only reduced the reprogramming efficiency compared to the control, but also strikingly suppressed nuclear deformation-induced iN reprogramming (FIG. 3h in Song et al.). Additionally, in fibroblasts pre-treated with JIB-04 for 24 hours and then introduced into the 7-μm channels, the decrease of H3K9me3 by squeezing was much less than the group without JIB-04 treatment (Fig. S23 and S24 in Song et al.). These results suggest that the decrease in H3K9me3 levels was required for the mechanical squeezing effect on iN reprogramming, which might be mediated by HDMTs. On the other hand, pre-treatment with the DNA methyltransferase (DNMT) inhibitor, Decitabine (0.5 μM) (Fig. S20 and S25 in Song et al.), slightly enhanced iN reprogramming efficiency, and the pre-treatment with Decitabine before squeezing further enhanced squeezing-induced iN reprogramming (FIG. 3i in Song et al.), demonstrating that DNA demethylation also played a role in iN reprogramming.

To investigate whether the combined effects of suppressing H3K9me3 and DNA methylation could match the reprogramming efficiency induced by mechanical squeezing, BAM-transduced fibroblasts were either subjected to transient nuclear deformation or treated with different combinations and concentrations of Decitabine and Bix01294. As shown in Fig. S26 and S27, in Song et al. Bix01294 or Decitabine alone, at its optimal concentration, only increased iN conversion by about 2-fold. Interestingly, 0.2 μM Decitabine combined with 0.4 μM Bix01294 (1:2) significantly increased the iN reprogramming efficiency in comparison to other combinations tested and showed similar efficiency when compared with the mechanically squeezed group (FIG. 3j in Song et al.), suggesting that the suppression of H3K9me3 and DNA methylation may be the major mediators of microchannel-induced iN reprogramming efficiency.

We further investigated whether ion channels were involved in squeezing-induced mechanotransduction leading to improved reprogramming efficiency. As shown in Fig. S28-33 in Song et al., the inhibition of Na⁺, K⁺, and Ca²⁺ ion channels and the manipulation of extracellular pH during the squeezing process did not significantly affect the iN reprogramming efficiency.

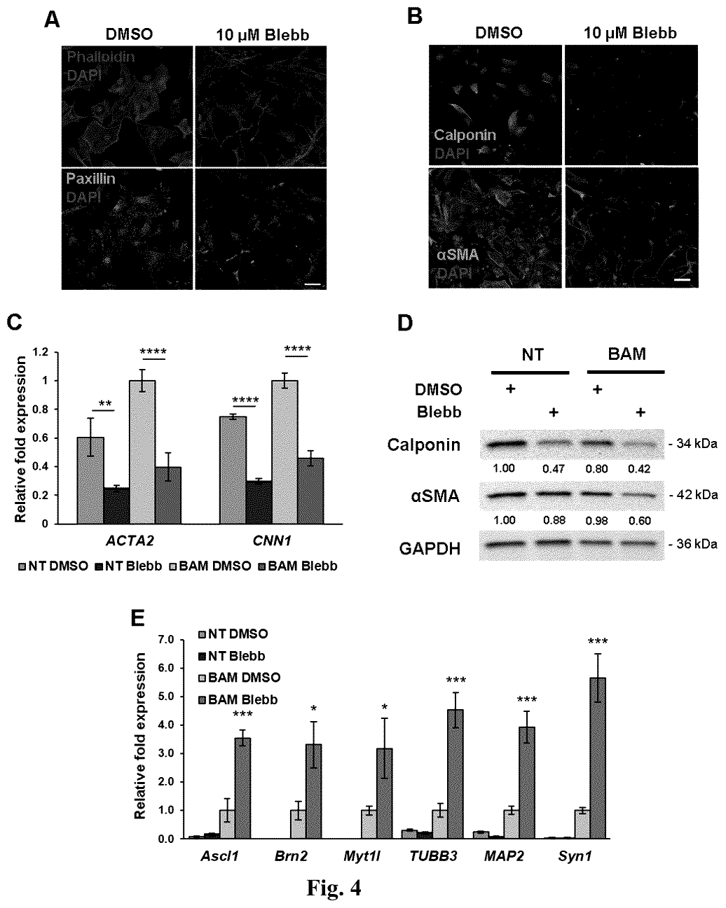

The observations that mechanical squeezing forced nuclear deformation and a decrease in the Young's modulus of the cells suggested that the structural changes of the nucleus could mediate mechanotransduction through the nuclear lamina. Indeed, lamin A/C staining showed that nuclear deformation by 7-μm microchannels induced a transient increase in nuclear wrinkling that lasted for at least 12 hours (FIG. 4a-b in Song et al.), more than that in cells passing through 200-μm channels (Fig. S34 in Song et al.). In addition, mechanical squeezing by 7-μm microchannels caused a transient decrease in lamin A/C assembly at the nuclear periphery (FIG. 4c in Song et al.) and a slight shift of lamin A/C to nucleoplasm within 3 hours (Fig. S35 in Song et al.). This increase in nucleoplasmic lamin A/C after mechanical squeezing might be attributed, at least in part, to changes in lamin A/C phosphorylation³⁵ as we observed a slight increase in phosphorylated lamin A/C at 30 minutes after mechanical squeezing (Fig. S36 in Song et al.). On the other hand, mechanical squeezing had no significant effect on the total protein levels of lamin A/C and lamin B1 (Fig. S36 in Song et al.). Interestingly, lamin B1 did not show a complete co-localization with lamin A/C following squeezing, especially within blebbing regions induced by 5-μm microchannels (Fig. S37 in Song et al.), and lamin B1 assembly at the nuclear periphery was not significantly affected by mechanical squeezing using 7-□m microchannels as lamin A/C (Fig. S38-39 in Song et al.). This finding is consistent with the reports that lamin A/C and lamin B1 form different structural layers³⁶ and that lamin A/C but lamin B1 regulates nuclear mechanics³⁷.

We further examined the changes of H3K9me3 and 5-mC in relation with lamin A/C following squeezing, and showed that the decrease of lamin A/C at nuclear periphery was accompanied by the decrease of H3K9me3 and 5-mc (Fig. S40, FIG. 3a-f, FIG. 4c in Song et al.). In addition, we examined the co-localization of the nuclear lamina and lamina-associated domains (LADs), which are enriched in heterochromatin (e.g. H3K9me3) and anchor chromatins to the nuclear lamina^{38,39}. We used a ^{m6}A-Tracer GFP reporter to label LADs⁴⁰, and used lamin B1 to label nuclear lamina as its distribution at nuclear periphery did not change significantly following squeezing (Fig. S38-39 in Song et al.). As shown in FIG. 4d and Fig. S41 in Song et al., after cells passed through 7-μm microchannels, part of the m6A-Tracer GFP signal was dislodged from the nuclear periphery, suggesting that a partial detachment between LADs and the nuclear lamina had occurred.

To investigate whether lamin disruption mediated nuclear deformation-induced epigenetic changes during iN reprogramming, we silenced lamin A/C by using a small interfering RNA (siRNA) in BAM-transduced fibroblasts 24 hours prior to mechanical squeezing (Fig. S42-S43 in Song et al.). As shown in FIG. 4e in Song et al., lamin A/C knockdown mimicked the effects of mechanical squeezing, which caused a lamin disassembly at nuclear periphery and a decrease in H3K9me3 and 5-mC (FIG. 4f, g in Song et al.). Moreover, lamin A/C knockdown enhanced the iN reprogramming efficiency of non-deformed cells to an extent similar to the cells that were mechanically squeezed but lamin A/C was not silenced (FIG. 4h in Song et al.). The iN cells derived after lamin A/C knockdown expressed MAP2 and Synapsin at 4 weeks after mechanical deformation (Fig. S44 in Song et al.). Taken together, these results suggested that lamin A/C played an important role in regulating the demethylation of histone and DNA induced by forced nuclear deformation.

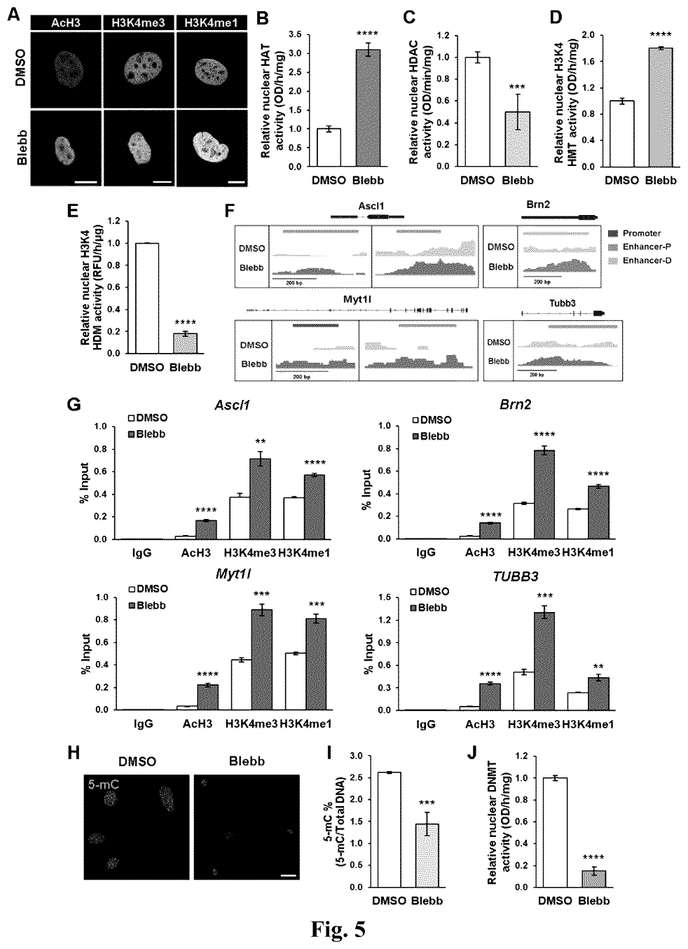

To determine whether microfluidic device-induced nuclear deformation could regulate the reprogramming of different cell types, we performed similar experiments by using macrophages transduced with BAM and fibroblasts transduced with Oct-4, Sox 2, KLF-4, and c-Myc (OSKM), respectively. Interestingly, we found that both iN reprogramming from macrophages and iPSC reprogramming from fibroblasts were significantly enhanced after nuclear deformation (FIG. 5a-b in Song et al.), suggesting that mechanical reprogramming can be utilized as a general approach to prime the epigenetic state of cells to promote cell reprogramming.

To scale up the mechano-preconditioning of cells for reprogramming, we developed a higher-throughput microfluidic device (HMD) containing 10 times more microchannels (400 microchannels) than the original microfluidic device (OMD) with 36 microchannels (FIG. 5c in Song et al.). The design was validated by fluid flow simulation, showing the velocity profile in different sizes of channels in the devices (Fig. S45 in Song et al.). HMD allowed the mechanical processing and collection of 10,000 cells within a minute, which was 5 times faster than OMD. Compared with OMD, this HMD significantly increased the yield of cell collection (FIG. 5d in Song et al.); HMD also maintained high cell viability and significantly improved iN efficiency (FIG. 5e-f in Song et al.).

### Methods

### Microfabrication of the Microfluidic Device

The molds of designed microfluidic devices for cell squeezing were fabricated via photolithography. A 15-μm thick layer of SU-8 2015 (Microchem Corporation, 3300 rpm) was spun coated onto a 4-inch silicon wafer, followed by standard photolithography process according to the manufacturer's instruction. Base and curing agent of polydimethylsiloxane (PDMS, Sylgard 184, Dow Corning) was mixed in a 10:1 weight ratio and degassed in a vacuum chamber for one hour to remove air bubbles before being poured onto the mold. After curing at 65° C. for 4 hours, the PDMS mold was punched to make inlets and outlets for tubing connections. The PDMS mold and pre-cleaned glass were bonded after treatment with oxygen plasma for 30 seconds. The bonded chips were baked at 65° C. for 10 minutes to enhance the bonding.

### Cell Isolation, Culture and Reprogramming

Fibroblasts were isolated from ear tissues of adult (1 month-old) C57BL/6 mice, Tau-EGFP reporter mice (Jackson Laboratory, 004779) and R26-M2rtTA;Col1a1-tetO-H2B-GFP compound mutant mice (Jackson Laboratory, 016836), and expanded in fibroblast medium: DMEM (Gibco, 11965), 10% fetal bovine serum (FBS; Gibco, 26140079) and 1% penicillin/streptomycin (GIBCO, 15140122). For all experiments, passage-2 cells were used and synchronized upon reaching 80% confluency using DMEM with 1% FBS for 24 hours before the transduction with viruses containing BAM constructs. The following day (day 0) the medium was changed to mouse embryonic fibroblasts (MEF) medium containing doxycycline (2 ng/ml, Sigma) to initiate the expression of the transgenes and thus, reprogramming. After 6 hours, transduced fibroblasts were passaged, and either subjected to microfluidic deformation using microchannels of various widths. Cells were then seeded onto glass slides coated with 0.1 mg/mL fibronectin (ThermoFisher, 33016015) overnight at a density of 3,000 cells/cm². Twenty-four hours later (day 1), cells were cultured in N2B27 medium: DMEM/F12 (Gibco, 11320033), N-2 supplement (Gibco, 17502048), B-27 supplement (Gibco, 17504044), 1% penicillin/streptomycin, and doxycycline (2 ng/ml), and half medium changes were performed every 2 days. On day 7 after microfluidic deformation, cells were fixed and stained for Tuj1 to determine the reprogramming efficiency. iN cells were identified based on positive Tuj1 staining and a neuronal morphology. The reprogramming efficiency was determined as the percentage of iN cells on day 7 relative to the number of the cells initially seeded. For long-term studies where maturation and functionality of the iN cells were examined, cells were kept in culture for 5 weeks. Reprogramming of iPSC from wild-type fibroblasts was performed as described previously¹⁸.

Macrophages for reprogramming experiments were derived from differentiated monocytes. Monocytes were isolated from the bone marrow of adult C57BL/6 mice, and expanded in monocyte medium: RPMI 1640 (Gibco, 11875093), 10% fetal bovine serum (FBS; Gibco, 26140079) and 1% penicillin/streptomycin (GIBCO, 15140122). The next day, macrophage-colony stimulating factor (M-CSF) (50 ng/ml, ThermoFisher, PMC2044) was added to the medium and cells were cultured for an additional 2 days. Cells were then washed 3 times with phosphate buffered saline (PBS) before transduction with viruses containing BAM constructs.

### Lentiviral Preparation and Transduction

Doxycycline-inducible lentiviral vectors for Tet-O-FUW-Brn2, Tet-O-FUW-Ascl1, Tet-O-FUW-Myt1l, and FUW-rtTA plasmids were used to transduce fibroblasts for ectopic expression of Brn2, Ascl1, Myt1L, GFP, and rtTA. The STEMCCA lentiviral vector was used for the ectopic expression of OSKM¹⁸. The Ascl1-eGFP lentiviral vector (Genecopoeia, MPRM39894-LvPF02) was used to monitor the activation of the Ascl1 promoter. Lentivirus was produced by using established calcium phosphate transfection methods, and Lenti-X Concentrator (Clontech, 631232) was utilized to concentrate viral particles according to the manufacturer's protocol. Stable virus was aliquoted and stored at −80° C. Fibroblasts were plated and synchronized for 24 hours before viral transduction in the presence of polybrene (8 μg/ml; Sigma, H9268). Cells were incubated with the virus for 24 hours before performing microfluidic deformation experiments.

### Cell Viability Assays

After cells passed through the micro-device, 10×10³ fibroblasts were plated and allowed to attach for 3 hours in a 96 well plate. Live and dead assays were performed using the LIVE/DEAD™ Cell Imaging Kit (Invitrogen, R37601) according to the manufacturer's protocol. Cells were incubated with an equal volume of 2× working solution for 15 μminutes at room temperature. Epifluorescence images were collected using a Zeiss Axio Observer Z1 inverted fluorescence microscope and analyzed using ImageJ.

Cell viability was assayed using the PrestoBlue® Cell Viability Reagent (Invitrogen, A13261) according to the manufacturer's protocol. Cells were incubated with the PrestoBlue Reagent for 2 hours. Absorbance was measured by a plate reader (Infinite 200PRO) at excitation/emission=560/590 nm. Results were normalized to control (i.e., cell passing through >200 □m channels) samples.

### DNA Damage Assays

After cells passed through the micro-device, 5×10³ fibroblasts were plated and allowed to attach for 3 hours in a 96 well plate. DNA damage assays were performed using the HCS DNA Damage Kit (Invitrogen, H10292) according to the manufacturer's protocol. Cells were fixed with 4% paraformaldehyde solution for 15 minutes at room temperature and permeabilized by 0.25% Triton® X-100 in PBS for another 15 minutes at room temperature. Cells were washed 3 times with PBS and incubated in 1% bovine serum albumin (BSA) solution for 1 hour, followed by pH2AX antibody (1:1000) for 1 hour at room temperature and then Alexa Fluor® 555 goat anti-mouse IgG (H+L) secondary (1:5000) with Hoechst 33342 (1:6000) for another 1 hour at room temperature after removing the antibody. Epifluorescence images were collected using a Zeiss Axio Observer Z1 inverted fluorescence microscope and analyzed using ImageJ. Results were normalized to control samples (i.e., cell passing through >200 □m channels) and cells treated with 200 nM lipopolysaccharide (LPS) served as a positive control.

### Immunofluorescence Staining and Microscopy

Samples collected for immunofluorescence staining at the indicated time points were washed once with PBS and fixed in 4% paraformaldehyde for 15 minutes. Samples were washed three times with PBS for 5 minutes each and permeabilized using 0.5% Triton X-100 for 10 minutes. After three subsequent PBS washes, samples were blocked with 5% normal donkey serum (NDS; Jackson Immunoresearch, 017000121) in PBS for 1 hour. Samples were incubated with primary antibodies (Supplementary Table S1 in Song et al.) in antibody dilution buffer (1% normal donkey serum (NDS)+0.1% Triton X-100 in PBS) for either 1 hour or overnight at 4° C. followed by three PBS washes and a 1-hour incubation with Alexa Fluor® 488- and/or Alexa Fluor® 546-conjugated secondary antibodies (Molecular Probes). Nuclei were stained with DAPI in PBS for 10 minutes. Epifluorescence images were collected using a Zeiss Axio Observer Z1 inverted fluorescence microscope and analyzed using ImageJ. Confocal images were collected using a Leica SP8-STED/FLIM/FCS Confocal and analyzed using ImageJ.

For DNA methylation staining, samples were fixed with ice-cold 70% ethanol for 5 minutes followed by three PBS washes. Samples were then treated with 1.5M HCl for 30 minutes and washed thrice with PBS. The immunostaining procedure proceeded from the donkey serum blocking step as aforementioned.

Average lamin and histone marker intensities per nuclei were quantified using an ImageJ macro. Gaussian blur, thresholding, watershed, and analyze particle functions were applied to the DAPI channel to create individual selections for each nucleus. This mask was applied to the corresponding stain image to measure the average fluorescence intensity within each nucleus.

### Chemical Treatment of Cells

To determine the role of H3K9 methylation in microfluidic device-induced iN reprogramming, BAM-transduced fibroblasts were treated with the H3K9 methyltransferase inhibitor Bix-01294 (Cayman chemical, 13124) or demethylase inhibitor JIB-04 (Cayman chemical, 15338) at the indicated concentrations for 24 hours prior to introduction into the microdevice. Parallel conditions with DMSO served as a control. The iN reprogramming efficiency was determined via Tuj1 staining 7 days after squeezing.

To determine the involvement of ion channels in microfluidic device-induced iN reprogramming, calcium channel blocker Amlodipine (Cayman chemical, 14838), potassium channel blocker Quinine (Cayman chemical, 23958) and sodium channel blocker procainamide (Cayman chemical, 24359) were used to inhibit calcium, potassium, and sodium ion channels, respectively. BAM-transduced fibroblasts were treated with small molecule blockers at the indicated concentrations for 12 hours prior to being introduced into the microfluidic device. Parallel conditions with DMSO served as a control. The iN reprogramming efficiency was determined via Tuj1 staining 7 days after squeezing.

To determine the effect of pH on forced nuclear deformation-induced iN reprogramming, BAM-transduced fibroblasts were treated with DMEM medium at different pH levels (pH=6.5, 7.5 and 8.5) for 1 hour prior to being introduced into the microdevice. The iN reprogramming efficiency was determined via Tuj1 staining at day 7 after squeezing.

### DNA Methylation Assay

After cells passed through the device, cells were collected and 10×10⁵ cells were plated in 60 mm dishes. At different time points, cells were trypsinized and DNA was extracted by Invitrogen PureLink Genomic DNA mini kit (Invitrogen, K1820-01). The 5-mC level was analyzed by the MethylFlash™ Global DNA Methylation (5-mC) ELISA Easy Kit (Epigentek, P-1030) according to the manufacturer's instructions. Briefly, 100 ng of sample DNA was bonded into the assay wells and incubated with a 5-mC detection complex solution for 60 minutes. Then color developer solution was added into assay wells, and the absorbance at 450 nm was measured by using a plate reader (Infinite 200Pro, 30050303).

### Reverse Transcription and Quantitative Polymerase Chain Reaction (RT-qPCR)

After cells passed through the device, cells were collected and 10×10⁵ cells were plated in 60-mm dishes. At different time points, TRIzol™ Reagent (Invitrogen, 15596026) was used to lyse cells, and RNA was isolated as described previously⁴⁹. After RNA extraction, ThermoScientific Maxima First Strand cDNA Synthesis Kit (ThermoFisher, K1641) was used for first-strand cDNA synthesis. Then qRT-PCR was performed to detect the gene expression levels of Ascl1 endogenous.

Lamin a siRNA Knockdown and m6A-Tracer Transfection

For Lamin A siRNA knockdown, 1×10⁶ cells were plated in 60-mm dishes for 24 hours. RNA interference was performed using ON-TARGETplus LMNA siRNA (Dharmacon, L-040758-00-0005), and transfections were carried out using Lipofectamine™ 3000 Reagent (ThermoFisher, L3000015) according to the manufacturer's protocol. Briefly, 250 μl Opti-MEM™ Medium (ThermoFisher, 31985062) was mixed with 7.5 μl Lipofectamine™ 3000 Reagent and incubated at 37° C. for 15 minutes. At the same time, 5 μg siRNA was diluted in 250 μl Opti-MEM™ Medium and incubated at 37° C. for 15 minutes. These two solutions were mixed and the DNA-lipid complexes to cells were added to 1.5 ml DMEM medium without FBS and penicillin/streptomycin and incubated at 37° C. for 12 hours. The media was then replaced with DMEM medium with 10% FBS and 1% penicillin/streptomycin. 1 day after transfection, RNA was isolated and qRT-PCR was performed to detect LMNA mRNA expression to determine whether Lamin A had been silenced.

For ^{m6}A-tracer transfection, 1×10⁶ cells were plated in 60-mm dishes for 24 hours followed by transfection with m6A-tracer GFP (AddGene, 139403) and DAM-lamin B1 (AddGene, 119764) using Lipofectamine™ 3000 Reagent (ThermoFisher, L3000015) according to the manufacturer's protocol. Briefly, 250 μl Opti-MEM™ Medium (ThermoFisher, 31985062) was mixed with 7.5 μl Lipofectamine™ 3000 Reagent and incubated at 37° C. for 5 minutes. At the same time, 5 μg DNA was diluted in 250 μl Opti-MEM™ Medium and incubated at 37° C. for 15 minutes. These two solutions were mixed and the DNA-lipid complexes to cells were added to 1.5 ml DMEM medium without FBS and penicillin/streptomycin and incubated at 37° C. for 24 hours. The media was then replaced with DMEM medium with 10% FBS and 1% penicillin/streptomycin. Three days after transfection, ^{m6}A-tracer GFP-labelled fibroblasts were subjected to microfluidic deformation experiments.

### Fluorescence Resonance Energy Transfer (FRET) Biosensor

Lentiviruses of H3K9me3 were produced from Lenti-X 293T cells (Clontech Laboratories, 632180) co-transfected with a pSin containing biosensor and the viral packaging plasmids pCMV-Δ8.9 and pCMV-VSVG using the ProFection Mammalian Transfection System (Promega, Cat. No. E1200). Viral supernatant was collected 48 hours after transfection, filtered with 0.45 μm filter (Sigma-Millipore). Primary fibroblasts were transduced with the virus, and the cells expressing the biosensor were sorted using flow cytometry (Sony, SH800). Images of the FRET experiment were taken with a Nikon Eclipse Ti inverted microscope equipped with a cooled charge-coupled device (CCD) camera, a 420DF20 excitation filter, a 450DRLP dichroic mirror, and two emission filters controlled by a filter changer (480DF30 for ECFP and 535DF35 for FRET) as described previously⁵⁰. The images were acquired, and the ECFP/FRET ratio was calculated and visualized by MetaFluor 7.8 (Molecular Devices).

Golden Nanorod (GNR) LNA Probe for mRNA

To detect Tubb3 mRNA expression in living cells after cells were passed through the device, cells were collected and plated in a 24 well plate at 2,000 cells/well, and GNR-LNA complexes specific to Tubb3 was added to culture media as described previously³². Briefly, GNR-LNA complexes were made by mixing 1.5 μl LNA Probe (10 μM), 2.5 μl golden nano-rod (GNR) and 46 μl Tris-EDTA buffer, and incubating at 37° C. for 15 minutes. The GNR-LNA complex solution (50 l) and fresh culture medium (450 μl) were then mixed and added to the cells. After 4-hour incubation, cells were washed with PBS, and fresh culture medium was added. Cells were incubated at 37 (in the dark for additional 60 minutes prior to performing live cell imaging. Epifluorescence images were collected using a Zeiss Axio Observer ZI inverted fluorescence microscope.

### AFM Measurement of Cell Mechanical Property

To determine the elastic modulus of cells after passing through the device, mechanical measurements of single cells were performed by using atomic force microscopy (AFM) (JPK Nanowizard 4a) with tipless cantilevers (NPO-10, Bruker Corp., USA), a high sensitive cantilever k=0.06 N/m, and sample Poisson's ratio of 0.499 at the UCLA Nano and Pico Characterization facility. During the measurement, cells were cultured on a glass-bottom dish with pre-warmed PBS and set on a temperature-controlled stage at 37° C. The force-distance curves were recorded and the elastic modulus of cells was calculated by NanoScope Analysis using the Hertz model.

### Western Blotting

Equal amounts of total protein (50 μg) from each sample were separated in a 10% SDS-PAGE gel and transferred to a PVDF membrane at 120 V for 2 hours at room temperature. The blot was blocked with 5% nonfat dry milk suspended in 1×TBS (25 mM Tris, 137 mM NaCl, and 2.7 mM KCl) for 1 hour. Membranes were incubated sequentially with primary antibodies and secondary antibodies. Bands were scanned using a densitometer (Bio-Rad) and quantified using the Quantity One 4.6.3 software (Bio-Rad).

### Statistics

All data are presented as mean±one standard deviation, where sample size (n)≥3. Comparisons among values for groups greater than two were performed by using a one-way analysis of variance (ANOVA) followed by a Tukey's post-hoc test. For two group analysis, a two-tailed, unpaired Student's t-test was used to analyze differences. For all cases, p-values less than 0.05 were considered statistically significant. Origin 2018 software was used for all statistical evaluations.

### Discussion

In this study, we demonstrate that the transient nuclear deformation in suspended cells decreases the methylation of H3K9me3 and DNA (5-mC), which primes the chromatin to a more permissive epigenetic state for reprogramming. This phenomenon is independent of complex microenvironmental factors such as extracellular matrix, cell-cell adhesions and pH, and may be generalized to various cell types as we have shown for fibroblasts and macrophages. It is important to note that this millisecond mechanical perturbation induces a transient change in epigenetic state within a 24-hour time window, allowing an earlier and more efficient activation of neuronal genes in heterochromatin, with global heterochromatin marks returning to the basal level afterwards.

Mechanical squeezing can increase the efficiency of reprogramming adult fibroblasts into iN cells (to ˜20%), which is much higher than using transgenes alone (2-3%), suggesting that mechanical factors can help overcome the epigenetic barrier during the reprogramming process 9. It is worth noting that, although the combination of chemical inhibitors for H3K9 and DNA methylation may reach the same iN efficiency as mechanical squeezing, the chemical inhibitors require hours of treatment and induce significant DNA damage (˜17% cells), while mechanical squeezing causes low DNA damage (˜3%), and in contrast, promoted cell proliferation (Fig. S46-S47 in Song et al.). In addition, squeezing did not significantly affect the activities of HMTs and DNMTs as compared with the chemicals (Fig. S48 in Song et al.), suggesting that this mechanical squeezing modulates H3K9me3 and 5-mc through different mechanisms. Furthermore, although mechanical squeezing did not induce a global change in AcH3 (Fig. S17 in Song et al.), the specific inhibition of histone deacetylase by valproic acid (VPA) slightly increased iN reprogramming efficiency, which was additive to mechanical squeezing (Fig. S49-51 in Song et al.), suggesting that the enhancement of an open-chromatin state may further facilitate iN reprogramming. Additionally, we found that squeezing did not promote chemical-induced iN reprogramming (Fig. S52 in Song et al.) via small molecule compounds (Forskolin, ISX-9, CHIR99021 and IBET-151)⁴¹, which may be explained by the fact that chemical-induced reprogramming process requires a complex signaling network to activate neuronal genes and different kinetics and time period for epigenetic modulations; in contrast, squeezing provides a 24-hour time window with a suppression of heterochromatin to enhance the bindings of reprogramming transcriptional factors to activate endogenous neuronal genes. Taken together, our findings suggest that squeezing provides some advantages over chemicals and may regulate chromatin organization differently than chemicals.

FRET experiments show that nuclear deformation downregulates H3K9me3 within minutes, which is accompanied by nuclear wrinkling, lamin A/C disassembly at nuclear periphery and a decrease in cell stiffness. This is consistent with an earlier observation during cell mitosis⁴². Knocking down lamin A/C mimics the effects of mechanical squeezing, suggesting that the nuclear lamina plays an important role in this mechanotransduction process. Indeed, heterochromatin is anchored to nuclear lamina through LADs that are abundant for heterochromatin marks such as H3K9me3 and are repressive for gene expression^{38,43,44}. Mechanical force-induced nuclear deformation may partially induce nuclear lamina reorganization, wrinkling, lamin A/C disassembly at nuclear periphery (FIG. 4c and Fig. S35 in Song et al.) and a partial detachment of heterochromatin from the nuclear lamina (FIG. 4d and Fig. S41 in Song et al.), which relocates heterochromatin towards the interior of the nucleus, accompanied by the downregulation of heterochromatin marks. This transient biophysical modulation of epigenetic state appears to be universal and independent of cell type and reprogramming factors, as squeezing macrophages also enhances their reprogramming into neurons and transient nuclear deformation promotes fibroblast reprogramming into iPSCs.

The simplicity of this mechanical approach by squeezing suspended cells provides direct evidence on the effect of nuclear deformation on chromatin remodeling. We also examined the roles of other major mediators of mechanotransduction^{45,46} and showed that YAP and Piezo-1 did not play a major role in mediating microchannel-induced epigenetic changes and iN reprogramming. Yap translocation into nucleus upon cell adhesion was not affected by the squeezing process (Fig. S53 in Song et al.). In addition, the knockdown of Piezo-1 by siRNA had a negligible effect on various histone marks including H3K9me3, H3K27me3, AcH3, H3K27ac and 5-mC (Fig. S54-S56 in Song et al.), but slightly decreased the microchannel-induced iN reprogramming efficiency (Fig. S57 in Song et al.), suggesting that Piezo-1 may contribute to iN reprogramming independent of epigenetic modulation.

The transient nuclear deformation in suspended cells is distinctly different from cell culture models because this approach decouples nuclear deformation from various cellular structures in adherent cells and the mechanical loading is transient and active. For example, passive biophysical factors such as micro/nano topography and matrix stiffness not only cause nuclear deformation, but also induce changes in cell adhesions and cytoskeleton organization that affect many other cellular processes. Active mechanical loading such as magnetic twisting on the surface of adherent cells, although can regulate gene expression in euchromatin, appears insufficient to overcome the heterochromatin barrier⁴⁷, which could be explained by the lack of significant nuclear deformation and/or global chromatin reorganization. Stretching or compressing adherent cells may decrease or increase heterochromatin²¹⁻²³, and these different effects could be related to the different magnitudes and rates of nuclear perturbation by these mechanical stimuli and other confounding factors such as cell adhesions and polarity.

Another highlight of this work is the translation of mechanobiology findings into mechano-biotechnology for cell engineering. Microfabricated devices provide a well-controlled microenvironment and real-time process control with a minimal benchtop space requirement⁴⁸. Here we developed a scalable microfluidic device that can be used to continuously process and precondition cells. This microfluidic device with multiple constriction channels can be used to engineer a variety of cells such as fibroblasts, stem cells and immune cells, and to facilitate the conversion of cell types from one to another, which will have broad applications in regenerative medicine, disease modeling, and drug screening.

### EXAMPLE 1 REFERENCES

1. Aydin, B. & Mazzoni, E. O. Cell Reprogramming: The Many Roads to Success. Annu. Rev. Cell Dev. Biol. 35, 433-452 (2019).
2. Karagiannis, P. et al. Induced pluripotent stem cells and their use in human models of disease and development. Physiological Reviews 99, 79-114 (2019).
3. Wang, H., Yang, Y., Liu, J. & Qian, L. Direct cell reprogramming: approaches, mechanisms and progress. Nature Reviews Molecular Cell Biology (2021). doi:10.1038/s41580-021-00335-z
4. Pang, Z. P. et al. Induction of human neuronal cells by defined transcription factors. Nature 476, 220-3 (2011).
5. Ieda, M. et al. Direct reprogramming of fibroblasts into functional cardiomyocytes by defined factors. Cell 142, 375-386 (2010).
6. Pagliuca, F. W. et al. Generation of functional human pancreatic β cells in vitro. Cell 159, 428-439 (2014).
7. Szabo, E. et al. Direct conversion of human fibroblasts to multilineage blood progenitors. Nature 468, 521-6 (2010).
8. Huang, P. et al. Direct reprogramming of human fibroblasts to functional and expandable hepatocytes. Cell Stem Cell 14, 370-84 (2014).
9. Vierbuchen, T. et al. Direct conversion of fibroblasts to functional neurons by defined factors. Nature 463, 1035-41 (2010).
10. Hu, W. et al. Direct Conversion of Normal and Alzheimer's Disease Human Fibroblasts into Neuronal Cells by Small Molecules. Cell Stem Cell 17, 204-212 (2015).
11. Takahashi, K. & Yamanaka, S. A decade of transcription factor-mediated reprogramming to pluripotency. Nature Reviews Molecular Cell Biology 17, 183-193 (2016).
12. Zwerger, M., Ho, C. Y. & Lammerding, J. Nuclear mechanics in disease. Annu. Rev. Biomed. Eng. 13, 397-428 (2011).
13. Song, Y., Soto, J., Chen, B., Yang, L. & Li, S. Cell engineering: Biophysical regulation of the nucleus. Biomaterials 234, (2020).
14. Uhler, C. & Shivashankar, G. V. Regulation of genome organization and gene expression by nuclear mechanotransduction. Nature Reviews Molecular Cell Biology 18, 717-727 (2017).
15. Song, Y., Soto, J. & Li, S. Mechanical regulation of histone modifications and cell plasticity. Curr. Opin. Solid State Mater. Sci. 24, 100872 (2020).
16. Discher, D. E. et al. Matrix Mechanosensing: From Scaling Concepts in 'Omics Data to Mechanisms in the Nucleus, Regeneration, and Cancer. Annual Review of Biophysics 46, 295-315 (2017).
17. Miroshnikova, Y. A., Nava, M. M. & Wickstrom, S. A. Emerging roles of mechanical forces in chromatin regulation. Journal of Cell Science 130, 2243-2250 (2017).
18. Downing, T. L. et al. Biophysical regulation of epigenetic state and cell reprogramming. Nat. Mater. 12, 1154-62 (2013).
19. Wang, P. et al. WDR5 modulates cell motility and morphology and controls nuclear changes induced by a 3D environment. Proc. Natl. Acad. Sci. U.S.A 115, 8581-8586 (2018).
20. Tan, Y. et al. Matrix softness regulates plasticity of tumour-repopulating cells via H3K9 demethylation and Sox2 expression. Nat. Commun. 5, 4619 (2014).
21. Nava, M. M. et al. Heterochromatin-Driven Nuclear Softening Protects the Genome against Mechanical Stress-Induced Damage. Cell 181, 800-817.e22 (2020).
22. Damodaran, K. et al. Compressive force induces reversible chromatin condensation and cell geometry-dependent transcriptional response. Mol. Biol. Cell 29, 3039-3051 (2018).
23. Li, Y. et al. Biophysical regulation of histone acetylation in mesenchymal stem cells. Biophys. J. 100, 1902-9 (2011).
24. X, D. et al. High-throughput Nuclear Delivery and Rapid Expression of DNA via Mechanical and Electrical Cell-Membrane Disruption. Nat. Biomed. Eng. 1, (2017).
25. Denais, C. M. et al. Nuclear envelope rupture and repair during cancer cell migration. Science (80-.). 352, 353-358 (2016).
26. Paul, C. D., Mistriotis, P. & Konstantopoulos, K. Cancer cell motility: lessons from migration in confined spaces. Nat. Rev. Cancer 17, 131-140 (2017).
27. B, N. et al. Passive and facilitated transport in nuclear pore complexes is largely uncoupled. J. Biol. Chem. 282, 3881-3888 (2007).
28. Raab, M. et al. ESCRT III repairs nuclear envelope ruptures during cell migration to limit DNA damage and cell death. Science 352, 359-62 (2016).
29. Irianto, J. et al. DNA Damage Follows Repair Factor Depletion and Portends Genome Variation in Cancer Cells after Pore Migration. Curr. Biol. 27, 210-223 (2017).
30. Wapinski, O. L. et al. Hierarchical Mechanisms for Direct Reprogramming of Fibroblasts to Neurons. Cell 155, 621-635 (2013).
31. Wapinski, O. L. et al. Rapid Chromatin Switch in the Direct Reprogramming of Fibroblasts to Neurons. Cell Rep. 20, 3236-3247 (2017).
32. Wang, S., Riahi, R., Li, N., Zhang, D. D. & Wong, P. K. Single Cell Nanobiosensors for Dynamic Gene Expression Profiling in Native Tissue Microenvironments. Adv. Mater. 27, 6034-6038 (2015).
33. Song, Y. et al. Asymmetric Cell Division of Fibroblasts is An Early Deterministic Step to Generate Elite Cells during Cell Reprogramming. Adv. Sci. 8, (2021).
34. De Carvalho, D. D., You, J. S. & Jones, P. A. DNA methylation and cellular reprogramming. Trends in Cell Biology 20, 609-617 (2010).
35. Cho, S. et al. Mechanosensing by the Lamina Protects against Nuclear Rupture, DNA Damage, and Cell-Cycle Arrest. Dev. Cell 49, 920-935.e5 (2019).
36. B, N. et al. Concentric organization of A- and B-type lamins predicts their distinct roles in the spatial organization and stability of the nuclear lamina. Proc. Natl. Acad. Sci. U.S. A. 116, 4307-4315 (2019).
37. Lammerding, J. et al. Lamins A and C but not lamin B1 regulate nuclear mechanics. J. Biol. Chem. 281, 25768-80 (2006).
38. van Steensel, B. & Belmont, A. S. Lamina-Associated Domains: Links with Chromosome Architecture, Heterochromatin, and Gene Repression. Cell 169, 780-791 (2017).
39. Collas, P., Ali, T. M. L., Brunet, A. & Germier, T. Finding Friends in the Crowd: Three-Dimensional Cliques of Topological Genomic Domains. Front. Genet. 10, (2019).
40. J, K. et al. Single-cell dynamics of genome-nuclear lamina interactions. Cell 153, 178-192 (2013).
41. Li, X. et al. Small-Molecule-Driven Direct Reprogramming of Mouse Fibroblasts into Functional Neurons. Cell Stem Cell 17, 195-203 (2015).
42. Peng, Q. et al. Coordinated histone modifications and chromatin reorganization in a single cell revealed by FRET biosensors. Proc. Natl. Acad. Sci. 115, 201811818 (2018).
43. Guelen, L. et al. Domain organization of human chromosomes revealed by mapping of nuclear lamina interactions. Nature 453, 948-951 (2008).
44. Becker, J. S., Nicetto, D. & Zaret, K. S. H3K9me3-Dependent Heterochromatin: Barrier to Cell Fate Changes. Trends in Genetics 32, 29-41 (2016).
45. Elosegui-Artola, A. et al. Force Triggers YAP Nuclear Entry by Regulating Transport across Nuclear Pores. Cell 171, 1397-1410.e14 (2017).
46. Monroe, T. O. et al. YAP Partially Reprograms Chromatin Accessibility to Directly Induce Adult Cardiogenesis In Vivo. (2019). doi:10.1016/j.devcel.2019.01.017
47. Sun, J., Chen, J., Mohagheghian, E. & Wang, N. Force-induced gene up-regulation does not follow the weak power law but depends on H3K9 demethylation. Sci. Adv. 6, eaay9095 (2020).
48. Gill, N. K. et al. A scalable filtration method for high throughput screening based on cell deformability. Lab Chip 19, 343-357 (2019).
49. Sia, J., Yu, P., Srivastava, D. & Li, S. Effect of biophysical cues on reprogramming to cardiomyocytes. Biomaterials 103, 1-11 (2016).
50. L, L. et al. Integration of FRET and sequencing to engineer kinase biosensors from mammalian cell libraries. Nat. Commun. 12, (2021).

### Example 2: Reduction of Intracellular Tension and Cell Adhesion Promotes Open Chromatin Structure and Enhances Cell Reprogramming

The role of transcription factors and biomolecules in cell type conversion has been widely studied. Yet, it remains unclear whether and how intracellular mechanotransduction through focal adhesions and the cytoskeleton regulates the epigenetic state and cell reprogramming. Here, we show that cytoskeletal structures and the mechanical properties of cells are modulated during the early phase of induced neuronal (iN) reprogramming, with an increase in actin cytoskeleton assembly induced by Ascl1 transgene. The reduction of actin cytoskeletal tension or cell adhesion at the early phase of reprogramming suppresses the expression of mesenchymal genes, promotes a more open chromatin structure, and significantly enhances the efficiency of iN conversion. Specifically, reduction of intracellular tension or cell adhesion not only modulates global epigenetic marks, but also decreases DNA methylation and heterochromatin marks and increases euchromatin marks at the promoter of neuronal genes, thus enhancing the accessibility for gene activation. Finally, micro and nano topographic surfaces that reduce cell adhesions enhance iN reprogramming. These novel findings suggest that the actin cytoskeleton and focal adhesions play an important role in epigenetic regulation for cell fate determination, which may lead to novel engineering approaches for cell reprogramming.

Cell reprogramming enables the derivation of distinct cell types that are highly valuable for regenerative cell therapy, disease modeling and therapeutic discovery^{1,2}. Direct cell conversion provides a faster and more direct method of generating desired cell types from somatic cells^{3,4}. Previous studies have demonstrated that fibroblasts can be directly converted into other cell types such as neurons^{5,6} and cardiomyocytes⁷ by using transcription factors, microRNAs and biophysical factors⁸⁻¹¹. However, low conversion efficiency has limited the translation of direct reprogramming strategies for therapeutic purposes. In addition, the role of mechanotransduction through intracellular structures such as actin cytoskeleton and focal adhesions (FAs), during direct reprogramming, is poorly understood.

Increasing evidence indicates that FAs and the cytoskeleton play important roles in sensing and transducing extracellular biophysical signals to modulate intracellular signaling and cell functions¹²⁻¹⁴. FAs are large, multiprotein complexes that provide a physical link between the extracellular matrix (ECM) and the cytoskeleton. FAs can be modulated by biochemical and biophysical cues in the cellular microenvironment, and activate signaling pathways that regulate cytoskeletal organization in response to mechanical cues^{12,15}. In eukaryotic cells, the cytoskeleton, primarily composed of actin microfilaments, intermediate filaments and microtubules, spans the cytoplasm to provide a structural link between the cell nucleus and the ECM. It serves to spatially organize contents of the cell and facilitates cell movement and shape changes through the generation of forces¹⁶. These intracellular structures have been implicated in regulating the mechanical phenotype of cells during many physiological and disease processes¹⁷⁻¹⁹ Additionally, there is evidence that the physical coupling of the cell nucleus with the cytoskeleton can affect chromatin structure and regulate the epigenetic state, gene expression and cell function^{20,21}. Yet, how intracellular structures, such as the actin cytoskeleton and FAs, regulate direct cell reprogramming is still unclear. Furthermore, whether these intracellular structures modulate the epigenetic state to influence direct cell conversion remains unknown.

Here we investigated the role of intracellular tension transmitted through the cytoskeleton and cell adhesion in cell reprogramming using the conversion of fibroblasts into induced neuronal (iN) cells⁵ as a model. Our results demonstrate that the reduction of intracellular tension in the early phase of the reprogramming can enhance the efficiency of iN conversion by promoting a more open chromatin structure to facilitate the activation of neuronal genes.

### Results

### Intracellular Structures and Mechanical Properties of Cells are Modulated During the Early Phase of iN Reprogramming

To elucidate the role of the various intracellular structures during iN conversion, primary fibroblasts isolated from adult mice were transduced with doxycycline (Dox)-inducible lentiviral vectors encoding three key reprogramming factors, Brn2, Ascl1, and Myt1l (BAM), and seeded onto tissue culture polystyrene dishes coated with laminin the following day. As illustrated in FIG. 1A, Dox was added one day later (marked as day 0) to initiate the expression of the transgenes and cells were cultured in neuronal medium (i.e., N3 medium) from day 1 to the conclusion of the experiment. To gain insights into the morphological changes that fibroblasts undergo as they reprogram into neurons, we first examined how the actin cytoskeleton was altered during the early phase of iN reprogramming. Interestingly, immunofluorescence analysis revealed that by day 1 of the reprogramming process, actin assembled into a network with a cage-like structure around the nucleus, but this structure along with the majority of the cytoskeleton gradually disappeared by day 3 (FIG. 1B). To determine whether these structural changes resulted in differences in the mechanical phenotype of cells, the mechanical properties of BAM-transduced fibroblasts was measured at similar time points using high-throughput quantitative deformability cytometry (q-DC), in which the timescale of a cell to transit through a narrow constriction provides a metric for cell deformability. We found that cell transit time and stiffness increased by day 1 and was followed by a decrease on day 3, which coincided with the observed cytoskeletal changes (FIG. 1C-D). Utilizing atomic force microscopy (AFM) to measure cell stiffness yielded a similar trend, consistent with our q-DC findings, and demonstrated more profound differences in cell stiffness across the various time points (FIG. 1E). These changes in cellular mechanical properties were transgene-specific as transduction with green fluorescent protein (GFP) did not produce a similar effect (FIG. 1E). These results suggest that the actin cytoskeleton and mechanical properties of cells are modulated during the early phase of reprogramming, possibly playing a role in iN conversion.

### Ascl1 Plays a Dominant Role in Regulating the Cell Mechanical Phenotype Changes

Next, we sought to determine the transgene that was responsible for the observed changes in the cytoskeleton and mechanical phenotype by reprogramming fibroblasts with individual or various combinations of the transgenes. Immunofluorescence and Western blot analysis of cytoskeletal structures and focal adhesion proteins demonstrated that Ascl1 promoted the actin cage-like structure and paxillin expression at day 1 (FIG. 2A). Although less intact stress fibers were observed at day 3, paxillin expression remained high (FIG. 2A). On the other hand, the presence of paxillin-positive punctate and cell spreading appeared to decrease in BM- and BAM-transduced fibroblasts (FIG. 2A). Interestingly, AFM analysis revealed that Ascl1 was the critical transgene in modulating the mechanical phenotype of cells in the early phase of reprogramming as it induced changes in cell stiffness in a manner comparable to BAM (FIG. 2B). To elucidate whether these observations could extend to the transcript level, we performed RNA sequencing analysis of non-transduced and Ascl1-transduced fibroblasts on day 3. We found that the overexpression of Ascl1 led to the over-representation of genes in gene ontology (GO) categories related to the cytoskeleton and cell adhesion, such as actin cytoskeleton organization and actin-mediated cell contraction (FIG. 2C). Altogether, these results suggested that the increase in cytoskeleton assembly at the early phase of reprogramming could be attributed to Ascl1-induced expression of cytoskeletal proteins.

### Reduction of Cytoskeletal Tension Enhances iN Reprogramming

To examine whether these changes in cell stiffness, actin structure and expression of contractility-mediating genes were involved in iN reprogramming, we evaluated the effects of disrupting the cytoskeleton using small molecule inhibitors. For these experiments, BAM-transduced fibroblasts were reprogrammed as in FIG. 1A and chemical compounds known to perturb the cytoskeleton were added to the culture medium on day 1 to evaluate their effects on the reprogramming process. After 2 weeks, cultures were fixed and immunostained for neuronal 3-III tubulin (TUBB3). iN cells were identified based on displays of a typical neuronal morphology (defined as cells with a circular soma extending processes that are at least three times the length of the cell body) and positive TUBB3 expression. The reprogramming efficiency was determined as the percentage of TUBB3*-iN cells normalized to the number of cells plated at 24 hours post-seeding (i.e., day 0). First, we tested whether disruption of cytoskeletal contractility could influence the direct reprogramming of fibroblasts into neurons using blebbistatin, a non-muscle myosin II inhibitor²²⁻²⁴. We observed that iN cells could be derived in the absence and presence of blebbistatin (FIG. 3A). Interestingly, when fibroblasts were induced to reprogram in the presence of varying concentrations of blebbistatin, we observed a biphasic dose response, suggesting that the reduction of intracellular tension to an optimal level may promote iN conversion (FIG. 3B). In particular, treatment with 10 μM blebbistatin generated the highest number of iN cells and increased the reprogramming efficiency by approximately 4.5-fold compared to the control, therefore, this concentration was used in all subsequent experiments. This enhanced reprogramming efficiency by blebbistatin was also confirmed by using mouse fibroblasts isolated from Tau-EGFP mice, whereby cells expressing the neuronal marker, Tau, are concomitantly labeled with GFP. As Ascl1 has been identified as a pioneer factor for iN reprogramming and transduction of Ascl1 alone is sufficient to generate iN cells^{25,26}, we further investigated whether blebbistatin could influence iN conversion by Ascl1 induction. Remarkably, we found that blebbistatin treatment could generate significantly more iN cells from Ascl1-transduced fibroblasts relative to control (FIG. 3C), suggesting that inhibition of cell contractility could promote single-factor reprogramming.

To determine whether interference of other cytoskeletal structures that can regulate intracellular tension affected iN conversion, we examined the effects of other chemical inhibitors, including Y-27632 (a Rho-kinase inhibitor to prevent stress fiber formation and contraction)²⁷, nocodazole (disrupting the assembly and disassembly dynamics of microtubules)²⁸, jasplakinolide (stabilizing actin filaments)¹², and cytochalasin D (inhibiting F-actin polymerization). Consistently, inhibition Rho-kinase increased the yield of iN cells similar to blebbistatin, although to a lesser degree (FIG. 3D); in contrast, treatment with nocodazole, which compromised cell division and other functions, resulted in a reduction in the reprogramming efficiency (FIG. 3E). Moreover, treatment with jasplakinolide or cytochalasin D reduced the reprogramming efficiency relative to control (FIG. 3F), suggesting that maintaining a certain level of actin cytoskeleton might be required for iN reprogramming.

Next, we determined the minimum length of time necessary for the inhibitor to elicit an effect by reprogramming fibroblasts in the absence and presence of blebbistatin over timescales of 1 to 14 days. Surprisingly, we found that administering blebbistatin for the first 3 days was sufficient to enhance the reprogramming efficiency, suggesting that relaxation of intracellular tension might facilitate initiation of the reprogramming process. Indeed, treatment with blebbistatin during distinct phases, i.e., early [days 1-5], mid [day 5-9], and late [day 9-13] stages of reprogramming demonstrated that the effects of cytoskeletal disruption were most crucial during the early phase of reprogramming (FIG. 3G). Additionally, time course studies of the reprogramming process revealed that as early as day 5, blebbistatin treatment led to more efficient generation of TUBB3⁺ iN cells than the control, and this trend was evident throughout a longer time period.

To ensure that the reduction of cytoskeletal tension at the early stage of reprogramming did not affect neuronal properties, we assessed the maturation and functionality of the iN cells derived in the absence and presence of blebbistatin. Immunostaining analysis for mature neuronal markers revealed that iN cells expressed NeuN, microtubule-associated protein 2 (MAP2), and synapsin (FIG. 3H). Similar observations were found in Tau-EGFP-derived iN cells. Electrophysiological analysis indicated that the iN cells were functional, exhibiting spontaneous changes in membrane potential in response to current injection (FIG. 3I). Further analysis of several action potential properties showed no apparent difference for iN cells generated in the absence and presence of blebbistatin. Although there was no significant difference in the frequency of spontaneous excitatory postsynaptic currents (EPSCs), the EPSC amplitude was significantly higher in iN cells derived with blebbistatin. Similarly, we observed no significant difference in spontaneous inhibitory post synaptic currents (IPSCs) frequency, however, the amplitude of IPSC was significantly lower in blebbistatin-derived iN cells. Furthermore, iN cells were found to be of the GABAergic and glutamatergic subtypes, in agreement with a previous report⁵. Collectively, these results suggest that iN cells derived with reduced myosin II activity still show signs of being mature and functional.

### Cytoskeletal Disruption Modulates Fibroblast and Neuronal Marker Expression

To gain insights into the mechanism by which disruption of cell contractility enhanced the reprogramming efficiency, we first examined the effects of blebbistatin on cell morphology. We observed that blebbistatin induced dramatic changes in cell morphology and reduced cell spreading in fibroblasts treated with blebbistatin for 24 hours (FIG. 4A). In addition, there appeared to be fewer intact actin fibers in fibroblasts treated with blebbistatin. Upon observing that cell spreading was affected, immunostaining analysis of focal adhesion proteins showed there were fewer positive paxillin punctate in blebbistatin-treated cells, suggesting that cytoskeletal disruption may modulate focal adhesion assembly and, as a result, cell adhesion (FIG. 4A).

It has been proposed that cell reprogramming involves the suppression of the original cell phenotype and the activation of the target cell fate regulatory program²⁹ Thus, we investigated whether blebbistatin's mechanism of action involved the repression of the mesenchymal phenotype in fibroblasts. To test this, non-transduced fibroblasts were cultured with and without blebbistatin for 24 or 48 hours, respectively, followed by analysis of mesenchymal marker expression. Interestingly, we observed less calponin and α-smooth muscle actin (SMA)-positive cells after blebbistatin treatment and that the expression for both markers had decreased at the gene and protein level by day 3 (FIG. 4B-D). Similarly, this trend was evident in BAM-transduced fibroblasts (FIG. 4C, D). Quantitative real-time polymerase chain reaction (qRT-PCR) analysis also showed that blebbistatin downregulated the expression of other mesenchymal and fibroblast-associated genes, Eln and DCN, in non- and BAM-transduced fibroblasts.

Subsequently, we explored the effect of cytoskeletal disruption on the induction of the neuronal phenotype by performing qRT-PCR analysis to evaluate neuronal gene expression at day 5. We found that the expression of various neuronal genes, including the key reprogramming factors, were significantly increased in BAM-transduced fibroblasts treated with blebbistatin, as compared to the non-treated transduced cells (FIG. 4E). To determine whether blebbistatin influenced the expression of the transgenes at an earlier time point, we analyzed the expression of BAM at day 3. qRT-PCR analysis revealed that Ascl1 expression was upregulated by blebbistatin at day 3. On the contrary, Brn2 and Myt1l did not show such a drastic increase in the expression level, suggesting that the reduction of cytoskeletal tension may enhance iN conversion efficiency by modulating Ascl1 expression. Considering two additional transcription factors along with Ascl1 are included in the reprogramming cocktail to generate iN cells, we questioned if perhaps blebbistatin could induce the expression of Brn2 and Myt1l to promote single factor reprogramming. Utilizing qRT-PCR to analyze the changes in neuronal gene expression in Ascl1-transduced fibroblasts, we found that blebbistatin upregulated the expression of endogenous Brn2 and Myt1l. Moreover, the expression of several neuronal genes, including the master neuronal gene, NeuroD1, was also higher in Ascl1-transduced fibroblasts treated with blebbistatin. In short, reduction of intracellular tension may influence iN conversion by inducing cell morphological changes that could impact nuclear morphology and thus, chromatin architecture and gene expression.

### Reduction of Cytoskeletal Tension Promotes a More Opened Chromatin Structure Globally and Locally

As cells undergo dynamic changes in gene expression and drastic chromatin remodeling during iPSC reprogramming^{30,31}, we postulated that the reduction of cytoskeletal tension might alter the epigenetic state to promote iN conversion. Therefore, to determine the effect of cytoskeletal modulation on global chromatin organization, we performed immunofluorescence analysis of histone marks associated with open chromatin structure (i.e. histone H3 acetylation (AcH3), tri-methylated histone H3 on lysine 4 (H3K4me3), and mono-methylated histone H3 on lysine 4 (H3K4me1)) and indicative of heterochromatin (i.e. tri-methylated histone H3 on lysine 27 (H3K27me3), tri-methylated histone H3 on lysine 9 (H3K9me3)) in non-transduced fibroblasts cultured with and without blebbistatin. As shown in FIG. 5A, interestingly, blebbistatin-treated cells exhibited an increase in AcH3, H3K4me3, and H3K4me1 marks while heterochromatin marks H3K27me3 and H3K9me3 decreased, compared to control, suggesting that inhibition of cytoskeletal contractility can induce global epigenetic changes and furthermore, may promote a more open chromatin structure. Similar histone trends were observed in fibroblasts treated with the Rho-kinase inhibitor (Y-27632) but no other cytoskeletal disrupting compounds (i.e., nocodazole, cytochalasin D and jasplakinolide), suggesting that these histone changes might be attributed to changes in cytoskeletal tension.

Analysis of chromatin-modifying enzyme activity showed that blebbistatin treatment increased histone acetyltransferase (HAT) activity while reducing histone deacetylase (HDAC) activity (FIG. 5B-C), which could potentially lead to an increase in histone H3 acetylation and thus, gene activation. In addition, blebbistatin treatment increased the activity of H3K4-specific histone methyltransferase (HMT) and reduced histone demethylase (HDM) activity, thereby possibly promoting H3K4 methylation (FIG. 5D-E).

To directly determine whether intracellular tension reduction could alter chromatin accessibility at specific sites of chromatin, we performed the assay of transposase accessible chromatin sequencing (ATAC-seq) and found that blebbistatin treatment, indeed, could increase the accessibility at the promoter or enhancer regions of neuronal genes, including Ascl1, Brn2, Myt1l and Tubb3 (FIG. 5F). Interestingly, the reduction of cytoskeletal tension slightly decreased the accessibility at the promoters or enhancer regions of some mesenchymal genes. To elucidate whether the observed epigenetic changes in active chromatin marks were involved in regulating neuronal gene expression during iN reprogramming, we performed a chromatin immunoprecipitation-quantitative polymerase chain reaction (ChIP-qPCR) assay at the promoter regions of Ascl1, Brn2, Myt1l, and TUBB3. ChIP-qPCR analysis revealed significant increases in AcH3, H3K4me3 and H3K4me1 at all four promoter regions in blebbistatin-treated cells relative to DMSO (FIG. 5G). Further examination of other epigenetic mechanisms that may be affected by inhibition of intracelullar tension revealed a decrease in the abundance of DNA methylation marker, 5-methylcytosine (5-mC), in blebbistatin-treated cells relative to the control (FIG. 5H-I). These findings were consistent with a decrease in DNA methyltransferase (DNMT) activity after blebbistatin treatment (FIG. 5J). Altogether, these results suggest that cytoskeletal disruption can reduce DNA methylation and promote global and site-specific changes in histone H3 acetylation and methylation that are conducive for neuronal gene activation.

### Inhibition of Focal Adhesion Kinase Improves iN Cell Generation

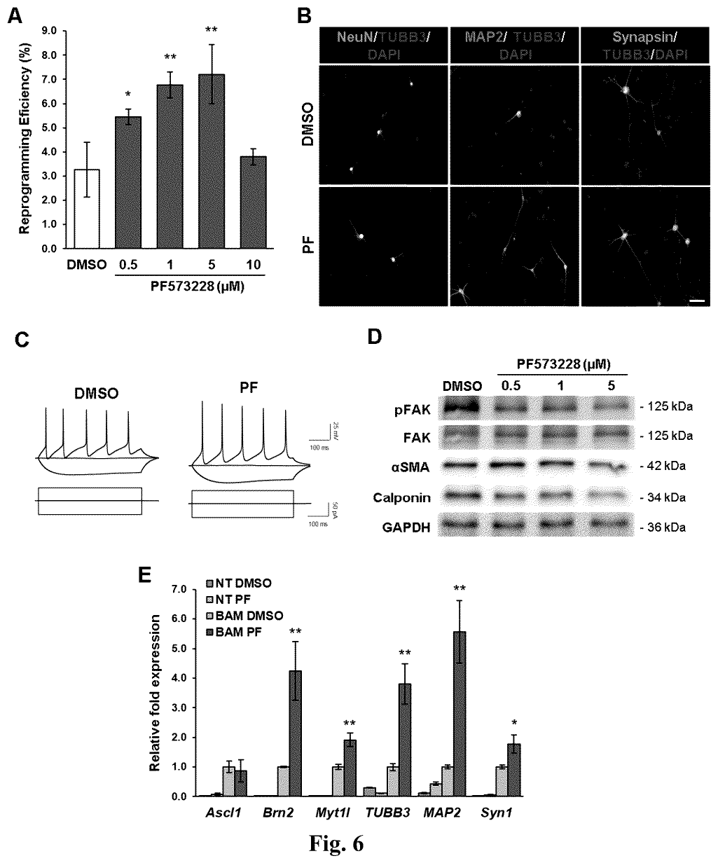

We then explored the modulation of intracellular tension via cell-ECM adhesions. Specifically, we examined the role of focal adhesion signaling by blocking the activity with the focal adhesion kinase (FAK) inhibitor, PF573228. Western blot analysis showed that PF573228 inhibited the phosphorylation of FAK (pFAK) at Tyrosine 397 (Tyr-397) in a dose-dependent manner and in addition, modulated downstream ERK signaling, demonstrating the specificity of the inhibitor. Immunostaining analysis also confirmed that PF573228 reduced pFAK expression in fibroblasts. Thereafter, BAM-transduced fibroblasts were induced to reprogram in the absence and presence of varying concentrations of PF573228 to test the effects of FAK inhibition on iN conversion. Interestingly, FAK inhibition significantly increased the reprogramming efficiency in a biphasic manner, similar to blebbistatin treatment, suggesting that a reduction of focal adhesions to an optimal level may facilitate iN conversion (FIG. 6A). Although FAK inhibition via PF573228 may influence ERK signaling, we found that the conversion efficiency was not significantly altered when BAM-transduced fibroblasts were treated with various doses of the ERK inhibitor, U0126, suggesting that the inhibition of FAK signaling, rather than ERK, plays a pivotal role in regulating iN reprogramming. Further characterization of the iN cells derived in absence and presence of the FAK inhibitor showed that these cells expressed neuronal markers, including NeuN, MAP2, and synapsin, and exhibited functional neuronal properties as assessed by electrophysiological analysis (FIG. 6B-C).

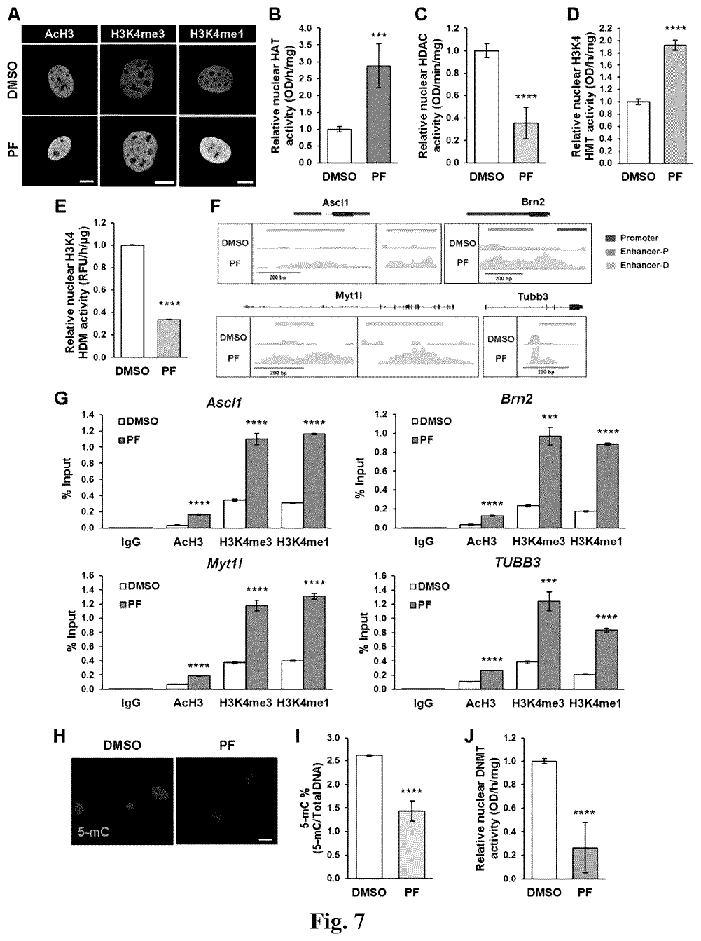

We further examined whether FAK inhibition could modulate mesenchymal and neuronal marker expression during iN conversion. Indeed, we found that calponin and αSMA expression decreased in BAM-transduced fibroblasts that were treated with PF573228 for 2 days (FIG. 6D). Conversely, neuronal gene expression was greater in fibroblasts transduced with BAM and cultured in the presence of 5 μM PF573228 at day 5, relative to the control (FIG. 6E). As with the reduction of intracellular tension, perturbations of cell adhesions also modulated the epigenetic state. Immunofluorescence analysis revealed that fibroblasts treated with the FAK inhibitor exhibited global increases in AcH3, H3K3me3 and H3K4me1 marks and a concurrent decrease in H3K9me3 and H3K27me3 compared to the control cells (FIG. 7A), which coincided with differences in HAT, HDAC, H3K4-specific HMT and H3K4-specific HDM activity (FIG. 7B-E). ATAC-seq analysis further revealed that FAK inhibition could increase the accessibility at the promoter or enhancer regions of neuronal genes Ascl1, Brn2, Myt1l and Tubb3 (FIG. 7F) and slightly decrease the accessibility of mesenchymal genes. These changes correlated well with localized site-specific epigenetic changes as ChIP-qPCR analysis revealed significant increases in AcH3, H3K3me3 and H3K4me1 at the promoter regions of Ascl1, Brn2, Myt1 and TUBB3 in PF573228-treated cells relative to DMSO (FIG. 7G). In addition, non-transduced fibroblasts treated with the FAK inhibitor for 24 hours displayed lower levels of DNA methylation, similar to blebbistatin treatment, as shown by a reduction in 5-mC marks and DNMT activity (FIG. 7H-J). These results demonstrate that a lower level of FAK enhances global and local epigenetic changes to promote iN reprogramming.

### Biomaterial-Mediated Reduction in Cell Adhesions Promotes iN Reprogramming

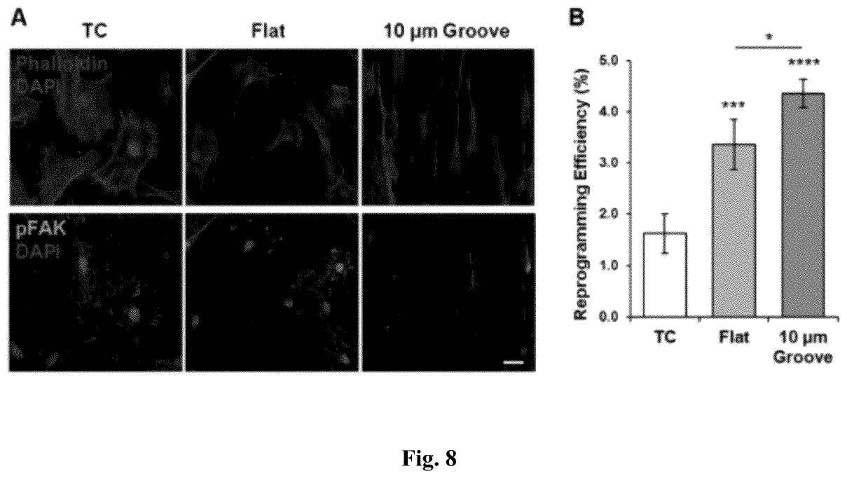

Given we had observed that reduction of intracellular tension and cell adhesion improved iN reprogramming, we postulated that modulating cell adhesion using biomaterials would produce a similar effect. When fibroblasts were grown on tissue-culture (TC) polystyrene wells or polydimethylsiloxane (PDMS) membranes with a flat surface or 10-μm microgrooves, we observed a decrease in stress fibers and phosphorylated FAK on PDMS membranes relative to TC wells, with the lowest levels on 10-μm microgrooves (FIG. 8A). Consistently, the iN reprogramming efficiency correlated inversely with stress fibers and FAK phosphorylation (FIG. 8B). In another example, when fibroblasts were cultured on binary colloidal crystals (BCCs) composed of spherical particle materials with distinct silica microparticle sizes (i.e., 5 μm versus 2 μm), we observed less cell spreading and paxillin-positive punctate in fibroblasts cultured on 2 μm BCCs, which coincided with an increase in the reprogramming efficiency (FIG. 8C-E). These findings demonstrate the potential of engineering biomaterials to modulate cell adhesion and thus, intracellular tension to enhance iN reprogramming.

### Methods

### Fibroblast Isolation, Culture, and Reprogramming

Mice utilized in these studies were housed under specific pathogen-free conditions and 12-hour light/12-hour dark cycles with a control of temperature (20-26° C.) and humidity (30-70%). All experiments, including breeding, maintenance and euthanasia of animals, were performed in accordance with relevant guidelines and ethical regulations approved by the UCLA Institutional Animal Care and Use Committee (Protocol #ARC-2016-036 and ARC-2016-101).

Ear tissues from adult B57BL/6 mice were isolated, minced and partially digested in Liberase™ (0.025 mg/ml, Roche) for 45 minutes under constant agitation at 37° C. Partially digested tissues were plated and fibroblasts were allowed to migrate out (passage 0). Isolated fibroblasts were expanded in MEF medium (DMEM+10% FBS [Corning] and 1% penicillin/streptomycin [GIBCO]) and used at passage 2 for all experiments. Fibroblasts from Tau-EGFP reporter mice (004779; The Jackson Laboratory) were isolated as described above.

After transduction, mouse fibroblasts were seeded onto multi-well tissue culture-treated polystyrene dishes (Falcon) coated with laminin (0.1 mg/ml, Corning) at 4,000 cells per cm². Twenty-four hours after seeding, the medium was replaced to MEF medium containing doxycycline (2 μg/ml, Sigma). The following day (i.e., day 1) the medium was changed to N3 medium (DMEM/F12 [GIBCO]+N2 supplement [Invitrogen]+B27 supplement [Invitrogen]+1% penicillin/streptomycin [Gibco]+ doxycycline [2 μg/ml, Sigma]) and the cultures were maintained in this medium for the duration of the experiments. For Ascl1-only reprogramming, N3 medium was further supplemented with BDNF (5 ng/ml, R&D systems) and GDNF (5 ng/ml, R&D systems) after day 7. For cytoskeletal and cell adhesion disruptions, Blebbistatin (Millipore), Y-27632 (20 μM; Cayman Chemical), Nocodazole (0.3 μM; Sigma), Cytochalasin D (1 μM; Sigma), Jasplakinolide (0.05 μM; Cayman Chemical) and PF573228 (Sigma) were administered in N3 medium on day 1 and for the first 7 days of reprogramming (unless stated otherwise) and used at the indicated concentrations. Culture medium was replenished every 2 days during reprogramming to maintain the activity of the small molecules. After culturing for the desired length (14 days for BAM and 21 days for Ascl1 only), the induced neuronal (iN) cells were analyzed and the reprogramming efficiency was determined.

### Lentiviral Production and Cell Transduction

Doxycycline-inducible lentiviral vectors for Tet-O-FUW-Ascl1, Tet-O-FUW-Brn2, Tet-O-FUW-Myt1l, Tet-O-FUW-GFP, and FUW-rtTA plasmids were used to transduce fibroblasts for ectopic expression of Ascl1, Brn2, Myt1l, GFP and rtTA. Lentivirus was made using established calcium phosphate transfection methods. Viral particles were collected and concentrated using Lenti-X Concentrator (Clontech) according to the manufacturer's protocol. Stable virus was aliquoted and stored at −80° C. For viral transduction, fibroblasts were seeded and allowed to attach overnight before incubation with the virus and polybrene (8 μg/ml, Sigma) for 24 hours. After incubation, transduced cells were reseeded onto laminin-coated tissue culture dishes.

### Immunofluorescent Staining and Quantification

For immunostaining, cells were fixed with 4% paraformaldehyde (Electron Microscopy Sciences), permeabilized with 0.5% Triton-X-100 (Sigma), and blocked with 5% donkey serum (Jackson Immunoresearch) in phosphate buffered saline (PBS). For actin-cytoskeleton staining, samples were incubated with fluorescein isothiocyanate-conjugated phalloidin (Invitrogen) for 1 hour. Primary antibodies were incubated for 1 hour at room temperature or overnight at 4° C., followed by 1-hour incubation with Alexa 488 and/or Alexa 546-labeled secondary antibodies (Molecular Probes). Nuclei were stained with 4,6-diamino-2-phenylindole (DAPI) (Invitrogen).

Two to three weeks after the addition of doxycycline, cultures were fixed and immunostained for neuronal beta-III tubulin (TUBB3). iN cells were quantified using a Zeiss Axio Observer.D1 and identified based on displays of a typical neuronal morphology (defined as cells with a circular cell body containing a neurite that is at least three times the length of the cell body) and positive TUBB3 expression, as previously described⁵. The reprogramming efficiency was determined by as the percentage of TUBB3⁺ iN cells in each condition normalized to the number of cells plated at 24 hours post-seeding. Epifluorescence images were collected using a Zeiss Axio Observer.D1, Zeiss Axio Observer.Z1, and ImageXpress Micro XLS System (Molecular Devices), whereas confocal images were acquired using a Zeiss LSM710 microscope and Leica SP8 Confocal Laser Scanning microscope.

Quantification of histone intensity per nuclei was performed using an ImageJ macro. DAPI-stained nuclei were segmented using gaussian blur, thresholding, watershed, and analyze particle functions to identify individual nuclei. This mask was applied to the corresponding stained fluorescence channel to quantify the average fluorescence intensity within each nucleus.

Quantitative Deformability Cytometry (q-DC)

To perform Quantitative Deformability Cytometry (q-DC), standard soft lithography methods were used to fabricate microfluidic channels in polydimethylsiloxane (PDMS). A mixture of 10:1 ratio of base to crosslinker (Sylgard 184, Dow Corning) was poured onto a master wafer containing bifurcating channels⁴². After curing, the PDMS device layer was bonded to a No. 1.5 glass coverslip (Thermo Fisher) using plasma treatment (Plasma Etch, Carson City, NV). Within 48 hours of device fabrication, cell suspensions of 1×10⁶ cells/mL were driven through constrictions of 9 μm (width)×10 μm (height) by applying 69 kPa of air pressure. We captured images of cells during deformation through the constrictions using a CMOS camera with a capture rate of 1600 frames/s (Vision Research, Wayne, NJ) mounted on an inverted Axiovert microscope (Zeiss, Oberkochen, Germany) equipped with a 20×/0.4NA objective. To analyze the time-dependent shape changes of individual cells during deformation, we used a custom MATLAB (MathWorks, Natick, MA) code (https://github.com/rowatlab)⁴². To determine the mechanical stresses applied to individual cells, we used devices that had been calibrated with agarose particles of defined elastic modulus as previously described⁴³. Stress-strain curves were obtained for single cells and a power-law rheology model was subsequently fitted to the data to yield measurements of elastic modulus, fluidity, and transit time.

### Atomic Force Microscopy (AFM)

To analyze the mechanical property of cells during the direct reprogramming of fibroblasts into neurons, mechanical measurements of single cells were performed using atomic force microscopy (AFM) (Bruker BioscopeResolve, Bruker Corp., USA) with silicon tipless cantilevers (NPO-10, Bruker Corp., USA), a high sensitive cantilever k=0.06 N/m, and sample Poisson's ratio of 0.499 at the UCLA Nano and Pico Characterization Facility. Fibroblasts were transduced with individual or different combinations of the transgenes and then we measured the cell stiffness at various time points during the reprogramming process (e.g. days 0, 1, and 3), wherein for each condition at least 30 cells were analyzed. During the measurements, cells were cultured on a glass bottom dish with pre-warmed PBS and set on a temperature-controlled stage at 37° C. The force-distance curves were recorded and the elastic modulus of cells was calculated by NanoScope Analysis (Bruker Corp., USA) using the Hertz model as the Fit Model. Similar AFM measurements were also conducted on control samples of non-transduced and GFP-transduced fibroblasts.

### Electrophysiology

For functional assessment of the iN cells, patch-clamp electrophysiology analysis was performed. All experiments were conducted at room temperature (22° C.-24° C.). All reagents were purchased from Sigma-Aldrich unless otherwise specified. Whole-cell recording was made from neurons using a patch clamp amplifier (MultiClamp 700B, Axon Instr.) under infrared differential interference contrast optics. Microelectrodes were made from borosilicate glass capillaries, with a resistance of 4-5 MW. For recording action potentials, cells were held at −70 mV in a voltage-clamp mode. The intracellular solution for whole-cell recording of EPSPs and action potentials contained (in mM) 140 potassium gluconate, 5 KCl, 10 HEPES, 0.2 EGTA, 2 MgCl₂, 4 MgATP, 0.3 Na₂GTP and 10 Na₂-phosphocreatine, pH 7.2 (adjusted with KOH).

For recording spontaneous EPSCs (sEPSCs), cells were pre-treated with the extracellular bath solution containing 50 μM picrotoxin (Tocris) to exclude an inhibitory synaptic activity and held at −70 mV in a voltage-clamp mode with the intracellular solution containing (in mM) 130 CsMeSO₄, 7 CsCl, 10 HEPES, 1 EGTA, 4 MgATP, 0.3 Na₂GTP, and 10 Na₂-phosphocreatine, pH 7.3 (adjusted with CsOH). After recording basal sEPSC responses for 5 min, 10 μM CNQX (Tocris) and 100 μM D,L-APV (Tocris) were co-treated to test whether sEPSCs were mediated by activation of both AMPA- and NMDA-type of glutamate receptors. For measuring spontaneous IPSC (sIPSCs), cells were pre-treated with the bath solution containing 10 μM CNQX and 100 μM D,L-APV and held at −70 mV with the intracellular solution containing (in mM) 137 CsCl, 10 HEPES, 1 EGTA, 4 MgATP, 0.3 Na₂GTP, and 10 Na₂-phosphocreatine, pH 7.3 (adjusted with CsOH). 50 μM picrotoxin was then treated to test a dependency of sIPSCs on GABA receptors after acquiring basal sIPSC responses for 5 min. Series resistance (10-25 MΩ) and input resistance (˜200 MΩ using potassium-based internal solution; 1-2 G2 using Cs-based internal solution) were monitored throughout the whole-cell recording or compared before and after sEPSC/IPSC recordings.

Off-line analyses of action potential properties (number, amplitude, half-width) and the amplitude and frequency of sEPSC and sIPSC were performed by using a threshold event detection function of the Clampfit software (Molecular Devices). Visualization of analysis results and their statistical tests were performed by using GraphPad Prism® 6.0 software.

Quantitative Reverse Transcriptase-Polymerase Chain Reaction (qRT-PCR)

RNA was isolated from samples using Trizol® (Ambion) according to the manufacturer's instructions. For cDNA synthesis, 500 ng of RNA was reverse transcribed using Maxima First Strand cDNA Synthesis Kit (ThermoFisher Scientific). Template DNA was amplified using Maxima SYBR Green/Fluorescein qPCR Master Mix (ThermoFisher Scientific) on a CFX qPCR machine (Bio-Rad). qRT-PCR data were analyzed using CFX Manager 3.1 (Bio-Rad) and gene expression levels were normalized to 18S.

### Chromatin Immunoprecipitation (ChIP)-qPCR

Halt™ Protease and Phosphatase Inhibitor Cocktail (ThermoFisher Scientific, 78442) was added to Cell Lysis (10 mM Tris-HCl pH 8.0, 85 mM KCl, 0.5% NP-40), Nuclei Lysis (10 mM Tris-HCl pH 7.5, 1% NP-40, 0.5% sodium deoxycholate, 0.1% SDS), and ChIP Dilution (0.01% SDS, 1.1% Triton X-100, 1.2 mM EDTA, 16.7 mM Tris-HCL pH 8.1, 167 mM NaCl) Buffers.

Three days post-Dox addition, 4×10⁶ BAM-transduced fibroblasts cultured in the absence and presence of 10 μM blebbistatin and 5 μM PF573228, respectively, for 2 days were fixed using 1% formaldehyde in PBS (Fisher Scientific, BP531) for 10 minutes. 125 mM Glycine was added for 5 minutes to quench excess formaldehyde, followed by 2 washes with cold 1×PBS. Cells were scraped and collected into microcentrifuge tubes and centrifuged at 800 g at 4° C. for 5 minutes. Upon removing the supernatant, cell pellets were snap-frozen in liquid nitrogen and stored at −80° C. The cells were then resuspended and lysed in Cell Lysis Buffer and resuspended in Nuclei Lysis Buffer prior to sonication using a Branson SFX250 Sonifier at 40% amplitude, 0.7 seconds on and 1.3 seconds off, for a total of 8 minutes. Samples were spun down at maximum speed in a 4° C. centrifuge and the supernatant was collected. 50 μL was removed from each sample and stored at 4° C. as a downstream internal control.

1.5 μg of normal rabbit IgG (Millipore, CS200581), anti-rabbit H3K4me3 antibody (Millipore, 04-473), anti-rabbit Histone 3 Acetylation (Millipore, 06-599) or anti-rabbit H3K4me1 antibody (Abcam, ab8895) were added to samples and incubated in a rotator overnight at 25 rpm in a 4° C. refrigerator. 20 μL of Piercem Protein A/G Magnetic Agarose Beads (ThermoFisher Scientific, 78610) were washed with Chip Dilution Buffer using a magnetic separation rack and added to each sample and incubated in a rotator for 2 hours at 25 rpm in a 4° C. refrigerator.

The supernatant was removed from the beads using a magnetic separation rack and the beads were subjected to a series of wash buffers: Low Salt Immune Complex Wash Buffer (0.1% SDS, 1% Triton X-100, 2 mM EDTA, 20 mM Tris-HCl pH 8.1, 150 mM NaCl), High Salt Wash Buffer (0.1% SDS, 1% Triton X-100, 2 mM EDTA, 20 mM Tris-HCl pH 8.1, 150 mM NaCl), LiCl Immune Complex Wash Buffer (0.25 M LiCl, 1% NP40, 1% sodium deoxycholate, 1 mM EDTA, 10 mM Tris-HCl pH 8.1), and Tris-EDTA (10 mM Tris-HCl pH8.0, 1 mM EDTA). The beads were resuspended in 50 μL of freshly prepared ChIP Elution Buffer (1% SDS, 0.1 M NaHCO₃) and placed in a 65° C. bath for 10 minutes. The supernatant was collected and this elution step was performed once more and the corresponding eluates were combined.

50 μL ChIP Elution Buffer was added to the stored internal controls from the post-sonication step. 20 μL of Reverse Crosslinking Salt Mixture (250 mM Tris-HCl pH 6.5, 62.5 mM EDTA pH 8.0, 1.25 M NaCl) with 5 mg/mL Proteinase K (Life Technologies, AM2548) and 62.5 ng/μL RNase A (AG Scientific, R-2000) was added to each sample and internal control and incubated at 65° C. overnight. Samples were purified using AMPure XP beads (Beckman Coulter Life Sciences, A63881) at 2× volume according to the manufacturer's instructions. qRT-PCR was performed on input samples, ChIP DNA samples and control samples using the primers and a CFX qPCR machine (Bio-Rad). Substantial fold enrichment was observed for each experimental condition. ChIP-qPCR data were analyzed by normalizing the DNA concentration to percent input using the relative standard curve method.

### RNA Sequencing

RNA was isolated from non-transduced and Ascl1-transduced fibroblasts at day 3 using Trizol® (Ambion) according to the manufacturer's protocol. A total of 500 ng total RNA was subjected to poly A selection using the Dynabeads® mRNA DIRECT™ kit (Invitrogen) followed by library preparation using the PrepX RNA-Seq for Illumina Library Kit (Wafergen) before sequencing on the HiSeq4000 (Illumina) at 50 single-read runs. Fastqc files were trimmed with trim galore v0.6.4 using default settings. Trimmed fastQ files were aligned to GRCm38 reference genome using STAR v2.7.1a⁴⁴ with default parameters and with “--quantMode GeneCounts” enabled to obtain the number of reads per gene. Gene counts were imported into R and differentially expressed genes were identified with DESEq2 v1.20.0⁴⁵ after fitting a linear model to account for the experimental variables. Gene ontology analysis was performed on the differentially expressed genes using the GOseq v1.32.0⁴⁶ package.

### Assay of Transposase Accessible Chromatin Sequencing (ATAC-Seq)

### Cell Preparation, Transposition Reaction, ATAC-Seq Library Construction and Sequencing

A total of 1,000,000 fibroblasts treated with vehicle control (DMSO), 10 μM blebbistatin or 5 μM PF573228 were collected after 2 hours and stored at −80° C. prior to sample processing. ATAC-seq was performed as described previously⁴⁷. In brief, frozen cells were thawed and washed once with PBS and then resuspended in 500 μL of cold PBS. The cell number was assessed by Cellometer Auto 2000 (Nexcelom Bioscience, Massachusetts, USA). 100,000 cells were then added to ATAC lysis buffer and centrifuged at 500 g in a pre-chilled centrifuge for 5 minutes. Supernatant was removed and the nuclei were resuspended in 50 μL of tagmentation reaction mix by pipetting up and down. The reactions were incubated at 37° C. for 30 minutes in a thermomixer with shaking at 1,000 r.p.m., and then cleaned up using the MiniElute reaction clean up kit (Qiagen). Tagmented DNA was amplified with barcoded primers. Library quality and quantity were assessed with Qubit 2.0 DNA HS Assay (ThermoFisher), Tapestation High Sensitivity D1000 Assay (Agilent Technologies), and QuantStudio® 5 System (Applied Biosystems). Equimolar pooling of libraries was performed based on QC values and sequenced on an Illumina® NovaSeq (Illumina, California, USA) with a read length configuration of 150 PE for [100]M PE reads (50M in each direction) per sample.

### Mapping, Peak Calling and Differential Peak Analysis

FASTQ files were trimmed with Trim Galore and cutadapt⁴⁸. Pair-ended reads were then aligned to the mouse reference genome (mm10) with Bowtie2⁴⁹. Mitochondrial reads and PCR duplicates were removed using SAMtools⁵⁰ and Picard (http://broadinstitute.github.io/picard/), respectively. Peaks were called over input using MACS3⁵¹, and only peaks outside the ENCODE blacklist region were kept. All peaks from all samples were merged and featureCount⁵² was used to count the mapped reads for each sample. Peaks that were up- or downregulated in different conditions were defined using DESeq2⁴⁵ with P_{adj}=0.001 as the threshold. Peaks located at cis-regulatory elements related to genes of interest (±5 kb region) were visualized using Integrative Genomics Viewer (IGV)⁵³ to demonstrate up- or downregulated differential peaks.

### Western Blotting

Fibroblasts were lysed and collected in Laemmli buffer (0.0625 mM Tris-HCl, 10% glycerol, 2% SDS, 5% 2-mercaptoethanol, 0.002% bromophenol blue) containing RIPA buffer (50 mM Tris-HCl, 150 mM NaCl, 1% Triton-X-100, 0.1% SDS, 10 mM NaF, 0.5% sodium deoxycholate) along with protease inhibitors (PMSF, Na₃VO₄ and Leupeptin) on ice. Protein lysates were centrifuged to pellet cell debris, and the supernatant was collected and used in further analysis. Protein samples were run using SDS-PAGE and transferred to polyvinylidene fluoride (PVDF) membranes. Membranes were blocked in 3% nonfat milk and incubated with primary antibodies overnight. Primary antibodies include pFAK, FAK, αSMA, Calponin, pERK, ERK, and GAPDH. Membranes were washed with Tris-Buffered Saline+0.05% Tween-20 and incubated with HRP-conjugated IgG secondary antibodies (Santa Cruz Biotechnologies) for one hour. Protein bands were visualized using Western Lightning™ Plus—Enhanced Chemiluminscence Substrate (Perkin Elmer Life & Analytical Sciences) and imaged on a ChemiDoc XRS system (Bio-Rad).

### Histone Acetyltransferase (HAT), Histone Deacetylase (HDAC), H3K4 Histone Methyltransferase (HMT), H3K4 Histone Demethylase (HDM) and DNA Methyltransferase (DNMT) Activity Assays

Nuclear protein extractions were isolated from 5×10⁵ fibroblasts treated with vehicle control (DMSO), 10 μM blebbistatin or 5 μM PF573228 for 2 hours using a nuclear extraction kit (EpiGentek, OP-0002), in accordance with the manufacturer's instructions. HAT, HDAC, H3K4 HMT, H3K4 HDM and DNMT activity were measured using the HAT activity/inhibition assay (EpiGentek, P-4003-048), HDAC activity/inhibition assay (EpiGentek, P-4034-096), Histone methyltransferase (H3K4 specific) activity/inhibition assay (EpiGentek, P-3002-1), Histone Demethylase (H3K4 specific) activity/inhibition assay (EpiGentek, P-3074-48), and DNMT activity/inhibition assay (EpiGentek, P-3009-048), respectively. Per the manufacturer's instructions, 20 μg of nuclear extract was added into the assay wells and incubated at 37° C. for 90 minutes. After adding the color developer solution, the absorbance was measured using a plate reader (Infinite 200Pro, 30050303) at 450 nm for all the assays with the exception of the Histone Demethylase (H3K4 specific) activity/inhibition assay where we measured the fluorescence using a fluorescence microplate reader at 530 EX/590 EM nm.

### DNA Methylation Assay

DNA was isolated from non-transduced fibroblasts treated with vehicle control (DMSO), 10 μM blebbistatin and 5 μM PF573228, respectively, for 24 hours using the PureLink Genomic DNA mini kit (Invitrogen) according to the manufacturer's instructions. To detect global DNA methylation (5-mC) levels in the samples, we utilized the MethylFlash Global DNA Methylation (5-mC) ELISA Easy kit (Epigentek) according to the manufacturer's protocol. 100 ng of DNA sample was utilized per reaction and after adding the color developer solution, the absorbance was measured using a plate reader (Infinite 200Pro, 30050303) at 450 nm.

### Microgroove Substrate Fabrication

Bioengineered substrates were fabricated as previously described³⁵. Briefly, PDMS membranes were fabricated using well established soft lithography procedures and sterilized using 70% ethanol for 10 minutes. PDMS membranes were plasma treated for 1 minute and coated with laminin (0.1 mg/ml, Corning) overnight to promote cell attachment. Fibroblasts were seeded onto PDMS membranes at 4,000 cells per cm² for subsequent experiments.

### Statistical Analysis

The data are presented as mean plus or minus one standard deviation, where n>3. The data corresponding to the q-DC, AFM and histone quantification experiments are displayed as box-and-whisker plots. The boxes are drawn with the ends at the quartiles, the median as a horizontal line in the box, the mean as a (+) symbol, and the whiskers extend from the minimum to maximum data point. Comparisons among values for groups greater than two were performed using a one-way or two-way analysis of variance (ANOVA) and differences between groups were determined using the following multiple comparison tests: Dunnett's, Tukey's and Sidak's post-hoc test. For comparison between two groups, a two-tailed, unpaired t-test was used. For all cases, p-values less than 0.05 were considered statistically significant. GraphPad Prism® 6.0 and GraphPad Prism® 8.0 software were used for all statistical analysis.

Our findings demonstrate, for the first time, that a reduction of cytoskeletal tension to an optimal level by using small molecule compounds, FAK inhibition and material engineering promotes a more open chromatin structure and enhances cell reprogramming. In particular, the reduction of cytoskeletal tension can suppress heterochromatin marks, and increase AcH3 and H3K4 methylation globally and at the promoter of neuronal genes (FIGS. 5 and 7), which in turn facilitates the reprogramming process. It is important to note that this reduction of tension is only required in the early phase of the reprogramming process (3-5 days, FIG. 3), suggesting that modulation of the epigenetic state is critical for initiating the reprogramming process but not necessarily for the maturation of neuronal phenotype. Interestingly, in comparison to blebbistatin, other actin and microtubule modulators such as cytochalasin D, jasplakinolide and nocodazole have opposite effects, i.e., decreasing euchromatin marks (e.g., AcH3) and inhibiting iN conversion (FIG. 3). While jasplakinolide enhances actin polymerization and nocodazole induces stress fibers, cytochalasin D inhibits actin polymerization. Therefore, these findings are consistent with the notion that a reduction of intracellular tension to an optimal level without compromising essential cell functions can promote epigenetic changes and cell reprogramming.

Our results suggest that intracellular tension regulates the epigenetic state through nuclear enzyme activities such as the increase of nuclear HAT and H3K4 HMT activity and the decrease in nuclear activity of HDAC and H3K4 HDM (FIGS. 5 and 7). One potential mechanism is the translocation of these enzymes between the cytoplasm and nucleus. Indeed, cytoskeleton tension can affect nuclear transport³². In addition, we show that the reduction of cytoskeletal tension causes the wrinkling and partial disassembly of nuclear lamina but does not induce a detachment of lamina-associated domain (LAD) of chromatin from the nuclear lamina. How these nuclear lamina changes regulate the transport of epigenetic enzyme activities remains to be determined. Furthermore, reducing intracellular tension suppresses the expression of mesenchymal genes in euchromatin (FIGS. 4 and 6), which may be attributed to both the decrease of accessibility at promoter and enhancer regions and the translocation of transcriptional factors³³³⁴. It is worth noting that mechanotransduction mechanisms in adherent cells may differ from cells being deformed in suspension. Cells in suspension have low-to-none intracellular tension, and transient squeezing has a direct physical effect on the nucleus such as the partial disruption of the nuclear lamina and LAD association, which causes a decrease in heterochromatin marks including H3K9me3 and DNA methylation¹¹.

Cytoskeletal tension can be modulated by cell adhesions and ECM. Since focal adhesions can transmit forces outside-in or inside-out between ECM and intracellular actin cytoskeleton, this represents an exciting opportunity to boost cell reprogramming by engineering the properties of cell adhesive substrates such as ligand density, stiffness and micro/nanotopography³⁵⁻⁴¹. Indeed, our results indicate that a reduction in cell spreading and focal adhesion signaling using micro/nano materials can facilitate the reprogramming process (FIG. 8). Taken together, our findings provide a potential explanation of the mechanotransduction mechanism by which cytoskeletal tension and cell adhesion can modulate reprogramming, and a rational basis for the design of novel biomaterials with biophysical properties that can be altered to provide an optimal level of cell adhesion and cytoskeletal tension that is conducive to direct reprogramming.

### EXAMPLE 2 REFERENCES

1. Grskovic, M., Javaherian, A., Strulovici, B. & Daley, G. Q. Induced pluripotent stem cells opportunities for disease modelling and drug discovery. Nat. Rev. Drug Discov. 10, 915 (2011).
2. Cherry, A. B. C. & Daley, G. Q. Reprogramming Cellular Identity for Regenerative Medicine. Cell 148, 1110-1122 (2012).
3. Graf, T. Historical origins of transdifferentiation and reprogramming. Cell Stem Cell 9, 504-16 (2011).
4. Morris, S. A. & Daley, G. Q. A blueprint for engineering cell fate: Current technologies to reprogram cell identity. Cell Research 23, 33-48 (2013).
5. Vierbuchen, T. et al. Direct conversion of fibroblasts to functional neurons by defined factors. Nature 463, 1035-41 (2010).
6. Pang, Z. P. et al. Induction of human neuronal cells by defined transcription factors. Nature 476, 220-3 (2011).
7. Ieda, M. et al. Direct reprogramming of fibroblasts into functional cardiomyocytes by defined factors. Cell 142, 375-86 (2010).
8. Xu, J., Du, Y. & Deng, H. Direct Lineage Reprogramming: Strategies, Mechanisms, and Applications. Cell Stem Cell 16, 119-134 (2015).
9. Lu, Y.-L. & Yoo, A. S. Mechanistic Insights Into MicroRNA-Induced Neuronal Reprogramming of Human Adult Fibroblasts. Front. Neurosci. 12, 522 (2018).
10. Wong, S. Y., Soto, J. & Li, S. Biophysical regulation of cell reprogramming. Curr. Opin. Chem. Eng. 15, 95-101 (2017).
11. Song, Y. et al. Transient nuclear deformation primes epigenetic state and promotes cell reprogramming. Nat. Mater. (2022). doi:10.1038/541563-022-01312-3
12. Martino, F., Perestrelo, A. R., Vinarský, V., Pagliari, S. & Forte, G. Cellular Mechanotransduction: From Tension to Function. Front. Physiol. 9, 824 (2018).
13. Muncie, J. M. & Weaver, V. M. The Physical and Biochemical Properties of the Extracellular Matrix Regulate Cell Fate. Curr. Top. Dev. Biol. 130, 1-37 (2018).
14. Shamsan, G. A. & Odde, D. J. Emerging technologies in mechanotransduction research. Curr. Opin. Chem. Biol. 53, 125-130 (2019).
15. Stutchbury, B., Atherton, P., Tsang, R., Wang, D.-Y. & Ballestrem, C. Distinct focal adhesion protein modules control different aspects of mechanotransduction. J. Cell Sci. 130, 1612-1624 (2017).
16. Fletcher, D. A. & Mullins, R. D. Cell mechanics and the cytoskeleton. Nature 463, 485-492 (2010).
17. Lin, H. H. et al. Mechanical phenotype of cancer cells: Cell softening and loss of stiffness sensing. Oncotarget 6, 20946-20958 (2015).
18. Toyoda, Y. et al. Genome-scale single-cell mechanical phenotyping reveals disease-related genes involved in mitotic rounding. Nat. Commun. 8, 1-11 (2017).
19. Clippinger, S. R. et al. Disrupted mechanobiology links the molecular and cellular phenotypes in familial dilated cardiomyopathy. Proc. Natl. Acad. Sci. U.S.A 116, 17831-17840 (2019).
20. Uhler, C. & Shivashankar, G. V. Regulation of genome organization and gene expression by nuclear mechanotransduction. Nature Reviews Molecular Cell Biology 18, 717-727 (2017).
21. Song, Y., Soto, J., Chen, B., Yang, L. & Li, S. Cell engineering: Biophysical regulation of the nucleus. Biomaterials 234, (2020).
22. Limouze, J., Straight, A. F., Mitchison, T. & Sellers, J. R. Specificity of blebbistatin, an inhibitor of myosin II. J. Muscle Res. Cell Motil. 25, 337-341 (2004).
23. Kovics, M., Tóth, J., Hetényi, C., Málnási-Csizmadia, A. & Sellers, J. R. Mechanism of Blebbistatin Inhibition of Myosin II. J. Biol. Chem. 279, 35557-35563 (2004).
24. Watanabe, M. et al. Blebbistatin, a myosin II inhibitor, suppresses contraction and disrupts contractile filaments organization of skinned taenia cecum from guinea pig. Am. J. Physiol. Cell Physiol. 298, C1118-C1126 (2010).
25. Wapinski, O. L. et al. Hierarchical Mechanisms for Direct Reprogramming of Fibroblasts to Neurons. Cell 155, 621-635 (2013).
26. Chanda, S. et al. Generation of induced neuronal cells by the single reprogramming factor ASCL1. Stem Cell Reports 3, 282-296 (2014).
27. Ishizaki, T. et al. Pharmacological properties of Y-27632, a specific inhibitor of rho-associated kinases. Mol. Pharmacol. 57, 976-983 (2000).
28. Samson, F., Donoso, J. a, Heller-Bettinger, I., Watson, D. & Himes, R. H. Nocodazole action on tubulin assembly, axonal ultrastructure and fast axoplasmic transport. J. Pharmacol. Exp. Ther. 208, 411-417 (1979).
29. Li, X. et al. Small-Molecule-Driven Direct Reprogramming of Mouse Fibroblasts into Functional Neurons. Cell Stem Cell 17, 195-203 (2015).
30. Buganim, Y., Faddah, D. a & Jaenisch, R. Mechanisms and models of somatic cell reprogramming. Nat. Rev. Genet. 14, 427-39 (2013).
31. Xu, Y. et al. Transcriptional Control of Somatic Cell Reprogramming. Trends Cell Biol. 26, 272-288 (2016).
32. Elosegui-Artola, A. et al. Force Triggers YAP Nuclear Entry by Regulating Transport across Nuclear Pores. Cell 171, 1397-1410.e14 (2017).
33. Foster, C. T., Gualdrini, F. & Treisman, R. Mutual dependence of the MRTF-SRF and YAP-TEAD pathways in cancer-associated fibroblasts is indirect and mediated by cytoskeletal dynamics. Genes Dev. 31, 2361-2375 (2017).
34. Dupont, S. & Wickström, S. A. Mechanical regulation of chromatin and transcription. Nat. Rev. Genet. 2022 2310 23, 624-643 (2022).
35. Downing, T. L. et al. Biophysical regulation of epigenetic state and cell reprogramming. Nat. Mater. 12, 1154-62 (2013).
36. Kulangara, K. et al. The effect of substrate topography on direct reprogramming of fibroblasts to induced neurons. Biomaterials 1-10 (2014). doi:10.1016/j.biomaterials.2014.03.034
37. Yoo, J. et al. Nanogrooved substrate promotes direct lineage reprogramming offibroblasts to functional induced dopaminergic neurons. Biomaterials 45, 36-45 (2015).
38. Morez, C. et al. Enhanced efficiency of genetic programming toward cardiomyocyte creation through topographical cues. Biomaterials 70, 94-104 (2015).
39. Sia, J., Yu, P., Srivastava, D. & Li, S. Effect of biophysical cues on reprogramming to cardiomyocytes. Biomaterials 103, 1-11 (2016).
40. Choi, B. et al. Stiffness of Hydrogels Regulates Cellular Reprogramming Efficiency Through Mesenchymal-to-Epithelial Transition and Stemness Markers. Macromol. Biosci. 16, 199-206 (2016).
41. Kong, Y. P. et al. A systems mechanobiology model to predict cardiac reprogramming outcomes on different biomaterials. Biomaterials 181, 280-292 (2018).
42. Nyberg, K. D. et al. The physical origins of transit time measurements for rapid, single cell mechanotyping. Lab Chip 16, 3330-3339 (2016).
43. Nyberg, K. D. et al. Quantitative Deformability Cytometry: Rapid, Calibrated Measurements of Cell Mechanical Properties. Biophys. J. 113, 1574-1584 (2017).
44. Dobin, A. et al. STAR: ultrafast universal RNA-seq aligner. Bioinformatics 29, 15-21 (2013).
45. Love, M. I., Huber, W. & Anders, S. Moderated estimation of fold change and dispersion for RNA-seq data with DESeq2. Genome Biol. 15, 1-21 (2014).
46. Young, M. D., Wakefield, M. J., Smyth, G. K. & Oshlack, A. Gene ontology analysis for RNA-seq: accounting for selection bias. Genome Biol. 11, 1-12 (2010).
47. Corces, M. R. et al. An improved ATAC-seq protocol reduces background and enables interrogation of frozen tissues. Nat. Methods 14, 959-962 (2017).
48. Martin, M. Cutadapt removes adapter sequences from high-throughput sequencing reads. EMBnet.journal 17, 10-12 (2011).
49. Langmead, B. & Salzberg, S. L. Fast gapped-read alignment with Bowtie 2. Nat. Methods 9, 357-359 (2012).
50. Danecek, P. et al. Twelve years of SAMtools and BCFtools. Gigascience 10, (2021).
51. Zhang, Y. et al. Model-based analysis of ChIP-Seq (MACS). Genome Biol. 9, 1-9 (2008).
52. Liao, Y., Smyth, G. K. & Shi, W. featureCounts: an efficient general purpose program for assigning sequence reads to genomic features. Bioinformatics 30, 923-930 (2014).
53. Robinson, J. T. et al. Integrative genomics viewer. Nat. Biotechnol. 29, 24-26 (2011).
54. Wang, P. Y., Pingle, H., Koegler, P., Thissen, H. & Kingshott, P. Self-assembled binary colloidal crystal monolayers as cell culture substrates. J. Mater. Chem. B 3, 2545-2552 (2015).
55. Wang, P. Y., Hung, S. S. C., Thissen, H., Kingshott, P. & Wong, R. C. B. Binary colloidal crystals (BCCs) as a feeder-free system to generate human induced pluripotent stem cells (hiPSCs). Sci. Reports 2016 61 6, 1-11 (2016).

### Example 3: 3D Spheroid Culture to Increase Cell Reprogramming Efficiency

Three-dimensional (3D) multicellular aggregates, termed “spheroids”, can affect cell-cell interactions, change the mechanical properties of microenvironment, and cause difference between cells on the spheroid surface and the core. It was not clear whether and how 3D spheroid culture affected direct neuronal reprogramming.

Spheroid Culture Enhances Direct Reprogramming of Fibroblasts into Neurons

Direct reprogramming involves both disruption of the existing regulatory network (generally early on) and establishment of another. The initial nonspecific disruption is often mechanistically associated with cell cycle regulation, cell senescence, chromatin inactivation, and genome stability. To assess the impact of mechanobiological changes associated with spheroid culture on direct reprogramming, primary human neonatal dermal fibroblasts (hNDFs) were transduced with doxycycline (dox)-inducible lentiviral vectors for the BAMN factors. After dox induction in monolayer to ensure unbiased activation of the transgenes, hNDFs were either plated onto Matrigel-coated cover slips as 2D controls or centrifuged in microwells to form 3D aggregates, or “spheroids”. The expression of neuron-specific β-tubulin III (Tuj1) was used as a marker of neuronal fate. Tuj1 expression began earlier in spheroids than in 2D culture, appearing as early as day two (two days after dox induction and one day after spheroid formation) (FIG. 9B-C).

To evaluate relative reprogramming efficiency, spheroids were replated after three days onto Matrigel-coated cover slips. We did not use enzymatic disaggregation because we found that neurons had disproportionate difficulty in recovering from and re-adhering after spheroid dissociation, perhaps due to the increased sensitivity of interconnected extended processes in spheroids to mechanical trituration. This was in agreement with previously published findings on dissociating neural precursor spheroids, which suffered from sluggish growth attributed to possible removal of vital receptors by enzyme-mediated dissociation, and even in the absence of enzyme dissociators, over 50% cell death after spheroid dissociation. At two weeks post-dox induction, monolayer iNs still displayed very few Tuj1+ cells. Spheroid iNs, in contrast, had dramatically improved neural conversion (4.06%, FIG. 9D), by over 67-fold (p<0.05, FIG. 1F).

### Spheroid Reprogramming Progresses in a Spatial Pattern

In order to assess the dynamic direct reprogramming process, we tracked the expression of Tuj1 over time. We knew that onset would be more rapid (as early as Day 2; FIG. 111B) and ultimately greater than in 2D (FIG. 11D-F), but what was unexpected was to see a consistent and distinct spatial pattern of Tuj1 expression. Rather than a gradual and spatially homogenous increase, expression began on the periphery of the spheroid, arguably moving somewhat inward over time but remaining concentrated on the exterior. Inhibition of TGF-β and BMP signaling enhanced the reprogramming, especially in the center of the spheroids.

### Example 4: Mechano-Epigenetic Mapping for Guided Gene Activation and Silencing

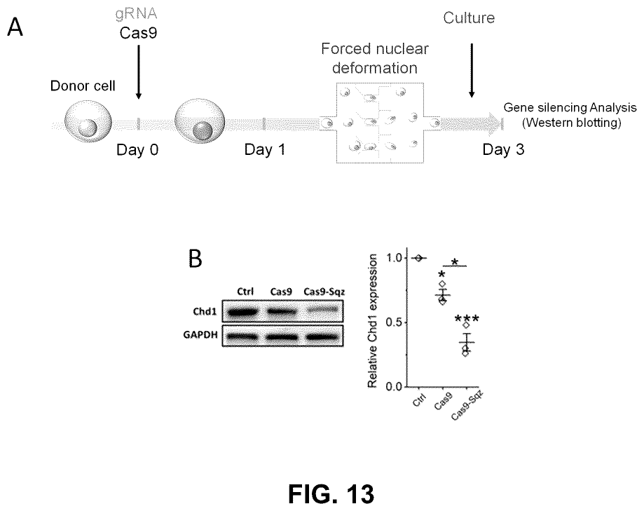

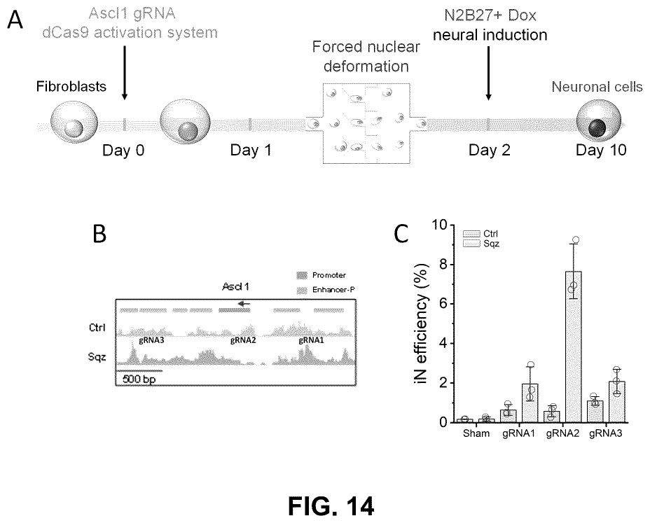

This embodiment of the invention describes a method that first generates a chromatin accessibility map following mechanical deformation of cell nucleus and a CRISPR design guide RNA (gRNA) for the opening sites at the promoter of target genes to increase the efficiency of CRISPR-mediated gene activation or silencing. This method is valuable for the target genes in heterochromatin that have low accessibility. First, cells were introduced into high throughput microfluidics devices with well-defined size of microchannels, and subjected to transient nuclear deformation. Three hours after cells passed through the channel, the cells were harvested for transposase-accessible chromatin with sequencing (ATAC-seq) to generate a genome-wide map of chromatin accessibility. Subsequently, gRNAs were designed to target the sites with an increase of chromatin accessibility at the promoter of the genes of interest. Finally, we transfected cells with gRNA and the construct expressing Cas9 or dCas9-activation complex by using Lipofectamine™ Stem Transfection Reagent, followed by a 24-hour incubation period. Then the transfected cells were subjected to nuclear deformation by using the high throughput microfluidics device. The mechanically-treated cells were collected and cultured for days, and the effects of gene activation or silencing were examined. Data from illustrative CRISPR methods of the invention is shown in FIGS. 13 and 14.

### Example 5: Matrix Stiffness Regulates Epigenetic State and Cell Reprogramming

The extracellular matrix stiffness has been studied as a monotonic or binary regulator of cell functions, and the mechanisms underlying epigenetic changes and cell reprogramming induced by matrix stiffness are not fully understood. Here we reveal that matrix stiffness serves as a biphasic regulator in the conversion of skin fibroblasts into induced neuronal (iN) cells, with the highest efficiency at an intermediate stiffness of 20 kPa, rather than on a soft surface known to facilitate neural differentiation. Epigenetic analysis indicates that histone 3 acetylation (AcH3) and the activity of histone acetyltransferase (HAT) in the nucleus also have biphasic responses to matrix stiffness, and HAT inhibition abolishes the effect of matrix stiffness on Ac-13 and iN conversion. These findings shed light on the mechanotransduction mechanism underlying epigenetic regulation by matrix stiffness, and have potential applications in cell engineering.

### Matrix Stiffness Serves as a Biphasic Regulator of iN Conversion

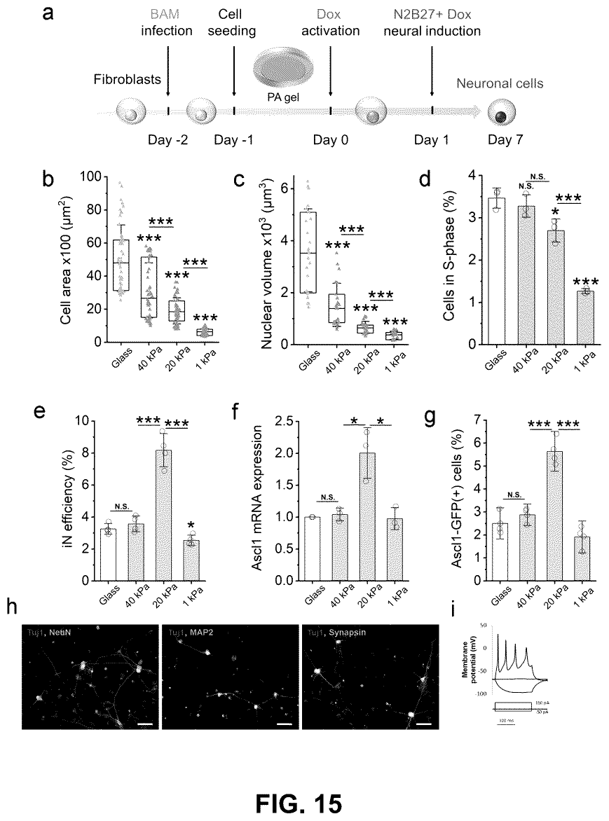

To determine the role of matrix stiffness on iN reprogramming, adult mouse fibroblasts were transduced with doxycycline-inducible lentiviruses containing the three reprogramming factors Ascl1, Brn2 and Myt1l (BAM), and then seeded onto polyacrylamide (PAAm) gels of various stiffness (40 kPa, 20 kPa, and 1 kPa) coated with fibronectin. Glass coverslips coated with the same ECM protein were used as a rigid glass surface control (FIG. 15a). As shown in FIG. 15b-c, both the cell area and nuclear volume of fibroblasts decreased as the stiffness of the PAAm gel decreased. After fibroblasts were seeded on PAAm gels for 48 hours, we found that the number of cells in the S-phase of the cell cycle decreased with matrix stiffness (FIG. 15d). These results suggest that matrix stiffness can significantly affect cell spreading and proliferation. To determine the effect of matrix stiffness on the direct conversion of fibroblasts into neurons, BAM-transduced fibroblasts were cultured on glass and PAAm gels of varying stiffness. After 7 days, cultures were fixed and immunostained for neuronal marker, neuron-specific class III β-tubulin (Tubb3) in order to identify iN cells and determine the reprogramming efficiency (FIG. 15a). Interestingly, we found that matrix stiffness had profound effects on the reprogramming efficiency, demonstrating a biphasic enhancement (FIG. 15e). Matrices of intermediate stiffness significantly enhanced the reprogramming efficiency, independent of ECM protein components (FIG. 15e). Interestingly, intermediate stiffness (˜20 kPa) significantly enhanced the mRNA level of pioneer factor Ascl1 compared to glass, 40 kPa and 1 kPa gels (FIG. 15f). To determine whether matrix stiffness could modulate the activation of endogenous Ascl1, fibroblasts were transduced with an Ascl1 promoter driven-GFP construct and reprogrammed for 2 days. We found a significant increase in the number of Ascl1 promoter-GFP+ cells in the intermediate stiffness group compared to glass and other stiffness groups (FIG. 15g). Further characterization of the derived cells by immunostaining and electrophysiology (patch clamp) analysis revealed that mature iN cells were obtained on PAAm gels (FIG. 15h-i). Taken together, these results suggest that an intermediate matrix stiffness may enhance iN reprogramming by promoting endogenous Ascl1 expression.

### Intermediate Stiffness Increases Histone Acetylation and Chromatin Accessibility for iN Conversion

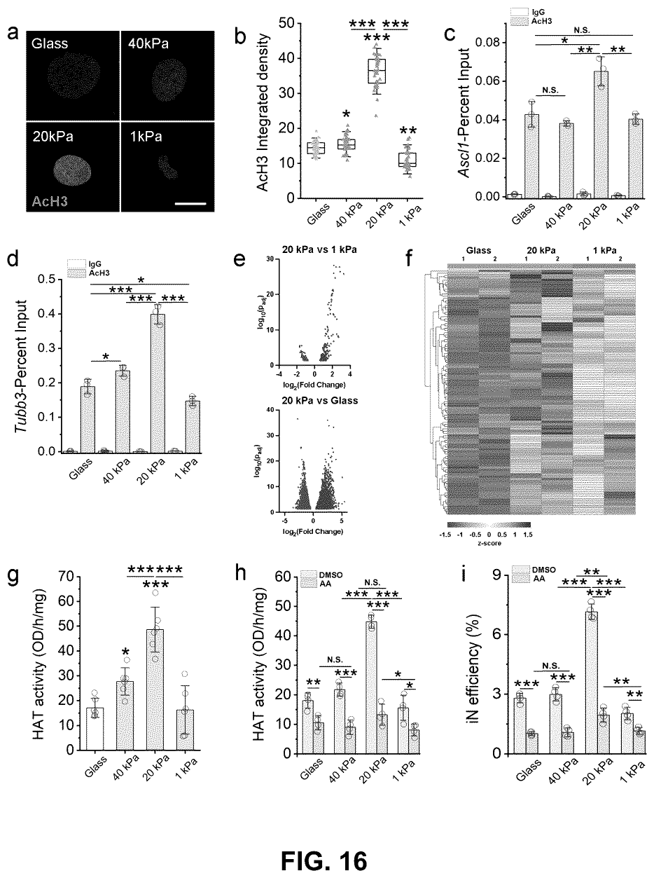

Previous studies have reported that epigenetic modifications, such as histone methylation, histone acetylation and DNA methylation, play an important role in cell reprogramming. To determine whether matrix stiffness may modulate iN reprogramming through global chromatin reorganization, we performed immunostaining of euchromatin marks AcH3, H3K27ac and 1H3K4me3, and heterochromatin marks H3K9me3, H3K27me3 and H4k20me3 in non-transduced fibroblasts cultured on gels of various stiffness. As shown in FIG. 16a-b, an intermediate stiffness of 20 kPa induced higher levels of AcH3 marks compared to stiffer and softer surfaces. On the other hand, no change was apparent in heterochromatin and other euchromatin marks examined. These results suggest that an intermediate stiffness may induce a more open chromatin stricture to facilitate cell reprogramming. Specifically, upon performing chromatin immunoprecipitation (ChIP)-qPCR at day 3, we found an increase in AcH3 at the promoter regions of Ascl1 and Tubb3, suggesting that an intermediate matrix stiffness can promote neuronal gene expression by modulating site-specific epigenetic changes (FIG. 16c-d). To directly determine whether matrix stiffness could alter chromatin accessibility, we performed the assay of transposase accessible chromatin sequencing (ATAC-seq), and found that cells on matrices with different stiffness (1 kPa, 20 kPa and glass) had distinguishable chromatin accessibility (FIG. 16e). Further analysis showed that an intermediate matrix stiffness significantly increased the accessibility of genomic regions of Ascl1-target genes in comparison to 1 kPa and stiff surfaces (FIG. 16f).

### Histone Acetyltransferase (HAT) Activity Accounts for the Stiffness Modulation of AcH3 and iN Reprogramming Efficiency

To further investigate how intermediate matrix stiffness promotes a more open chromatin state, we analyzed the activity of histone acetyltransferase (HAT), in fibroblasts cultured on PAAm of varying stiffness for 2 days. Quantification of HAT activity revealed that gels of intermediate stiffness increased HAT activity compared to stiffer and soft surfaces (FIG. 16g). We then used chemical inhibitors to test the relative contributions of these epigenetic regulators to iN conversion. Inhibition of HAT activity by anacardic acid inhibited the biphasic enhancement of HAT activity and the reprogramming efficiency (FIG. 16h-i). These results suggest that HATs play a major role in matrix stiffness-mediated iN conversion.

### Example 6: Viscoelasticity of Matrix Regulates Epigenetic State and Cell Reprogramming

While biochemical factors have been widely recognized as regulatory signals of cell reprogramming, how biophysical factors, e.g., mechanical properties of cell-adhesive matrices, regulate cell reprogramming are not well understood. Since extracellular matrix (ECM) has complex mechanical properties, including viscoelasticity, nonlinear elasticity, and plasticity, engineering synthetic matrices with tunable mechanical properties to investigate the mechanical regulation of cell reprogramming will provide crucial insights of cell fate determination during development and tissue regeneration. Therefore, we investigate how matrix viscoelasticity regulates cell reprogramming by employing hydrogels with independently tunable stiffness and viscoelasticity.

We used covalent and ionic crosslinking of alginate hydrogel to independently control the stiffness and viscoelasticity respectively, and adjusted the concentration of crosslinkers and the molecular weight of alginate polymers to fabricate matrix with defined mechanical properties. Mechanical characterization was performed to confirm the elastic moduli and stress relaxation properties of hydrogels by using three interconnected methods including atomic force microscopy (AFM), rheology measurement, and compression tests. Adult mouse fibroblasts transduced with doxycycline (DOX)-inducible lentiviral vectors containing three reprogramming factors (i.e., Brn2, Ascl1, and Myt1l, BAM) were then seeded onto hydrogels. Neuron-specific class III β-tubulin (Tuj1) expression of cells after one week will be examined to compare the reprogramming efficiency of fibroblasts on hydrogels with different properties. Mechanical regulation of epigenetic changes was examined by the analysis of histone modifications and chromatin accessibility.

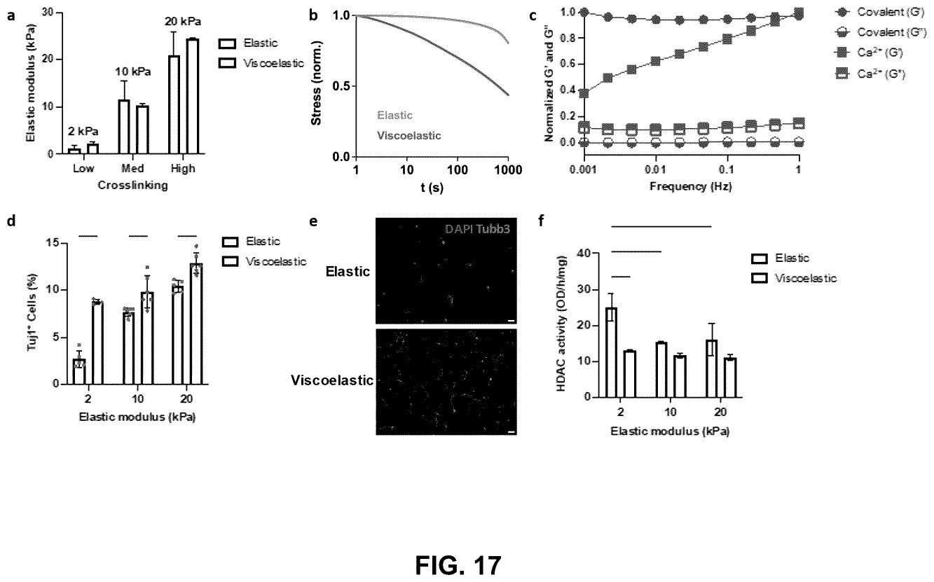

We constructed alginate hydrogels with different stiffness and stress relaxation behaviors by tuning the concentration of covalent and ionic crosslinkers (FIG. 17a). Rheology measurement and compression test results demonstrated that covalently crosslinked alginate hydrogels do not exhibit stress relaxation in response to deformation, while ionically crosslinked gels have time-dependent mechanical dynamics (FIG. 17b-c). To determine the effect of matrix stress relaxation on the direct conversion of fibroblasts into neurons, BAM-transduced fibroblasts were cultured on alginate hydrogels of tunable mechanical properties. After 7 days, cultures were fixed and immunostained for Tuj1 to identify iN cells and determine the reprogramming efficiency. We found that matrices with stress relaxation behaviors significantly enhanced the reprogramming efficiency compared to matrices without stress relaxation (FIG. 17d-e), especially at the low stiffness that facilitates neural maturation. Epigenetic analysis suggested that viscoelastic matrix promoted a more open chromatin structure for cell reprogramming. In addition, the analysis of histone deacetylase (HDAC) activity (FIG. 17f) suggested that the decrease of HDAC activity in the cells on viscoelastic matrix might account for the enhanced reprogramming efficiency.

By employing hydrogels that have similar viscoelastic properties to native tissue and ECM, our findings provide mechanistic insights of how mechanical cues regulate cell reprogramming, and will facilitate the development of innovative materials for cell engineering in vitro and in vivo.

All publications mentioned herein (e.g., those listed in the Examples above) are incorporated herein by reference to disclose and describe aspects, methods and/or materials in connection with the cited publications.

1. A microfluidic cell processing system comprising:
an inlet reservoir configured to receive cells;
an outlet reservoir to collect cells from the microfluidic system; and
at least one channel coupling the inlet reservoir to the outlet reservoir;
wherein:
the at least one channel is configured so that a mammalian cell contacts the channel and undergoes cellular and/or nuclear deformation as the cell moves from the inlet reservoir through the channel to the outlet reservoir.

2. The microfluidic cell system of claim 1, further comprising mammalian cells that are moved through the at least one channel and subjected to cell and nuclear deformation.

3. The microfluidic cell system of claim 1, wherein the at least one channel is not more than 3 μm, 7 μm or 10 μm in width.

4. The microfluidic cell system of claim 2, wherein the mammalian cells are somatic cells, stem cells, immune cells, induced pluripotent stem cells, and/or cells transfected or transduced with an exogenous nucleic acid or protein.

5. The microfluidic cell system of claim 4, further comprising an agent selected to modulate the physiology of the mammalian cells.

6. The microfluidic cell system of claim 5, wherein the agent is selected from a cytoskeleton inhibitor, an adhesion inhibitor, a TGF-β/Activin pathway inhibitor, and/or a BMP pathway inhibitor.

7. The microfluidic cell system of claim 4, wherein the mammalian cells comprise an exogenous nucleic acid or protein.

8. The microfluidic cell system of claim 1, wherein:
the at least one channel is at least 2 μm in width and not more than 200 μm in width for the cross-section; and/or
the at least one channel is configured to have an aspect ratio from 0.25 to 1; and/or
the at least one channel cross-section is polygonal, circular or elliptical.

9. The microfluidic cell system of claim 1, wherein the inlet reservoir, the outlet reservoir and the at least one channel are disposed on a polymer such as polydimethylsiloxane in a microfluidic chip configuration.

10. A method of mechanically deforming a mammalian cell and its nucleus comprising:
selecting the mammalian cell for mechanical deformation in a microfluidic cell culture system comprising:
an inlet reservoir configured to receive cells;
an outlet reservoir to collect cells from the microfluidic system; and
at least one channel coupling the inlet reservoir to the outlet reservoir;
wherein:
selecting the mammalian cell comprises determining the sizes of the mammalian cell and nucleus, and further selecting a dimension such as a width of the at least one channel in the microfluidic cell-deforming system; and
disposing the mammalian cell in the microfluidic cell culture system such that the mammalian cell contacts sides of the at least one channel so as to undergo cellular and/or nuclear deformation as the cell moves from the inlet reservoir through the at least one channel to the outlet reservoir, such that the cell and nuclear deformation cause changes in DNA/chromatin modification and organization.

11. The method of claim 10, wherein the mammalian cells are collected from the microfluidic system, and further cultured and induced to reprogram and/or differentiate in a cell culture system comprising a matrix for 2D or 3D cell culture.

12. The method of claim 11, wherein somatic cells are disposed in the inlet reservoir, collected from the outlet reservoir, and further cultured and reprogrammed into pluripotent stem cells.

13. The method of claim 11, wherein somatic cells are disposed in the inlet reservoir, collected from the outlet reservoir, and further cultured and reprogrammed into neuronal cells.

14. The method of claim 11, wherein the mammalian cells comprise an exogenous nucleic acid.

15. The method of claim 14, wherein the exogenous nucleic acid comprises DNA of a gene to be expressed, single guide RNA for gene targeting, and/or mRNA of a gene to be expressed.

16. The method of claim 10, wherein:
the size of the at least one channel in the microfluidic cell culture system is selected such that the mammalian cell contacts the channel and experiences transient disassembly of nuclear lamina as the cell moves from the inlet reservoir through the channel to the outlet reservoir; and/or
the cells undergo nuclear deformation for a time period between 0.1 milli-second and 100 seconds.

17. The method of claim 10, wherein nuclear deformation-induced chromatin accessibility is profiled using ATAC-sequencing in the mammalian cell genome.

18. A three dimensional (3D) culture system comprising mammalian cells configured as 3D spheroids, wherein the 3D spheroids are transfected or transduced with an exogenous nucleic acid or protein in one or more methods of cellular reprogramming, gene activation, gene silencing, gene editing or gene insertion.

19. The three dimensional (3D) culture system of claim 18, wherein the mammalian cells are cultured with a physiology modulating agent selected from a cytoskeleton inhibitor, an adhesion inhibitor, a TGF-β/Activin pathway inhibitor, and/or a BMP pathway inhibitor.

20. A biomaterial-based culture system, wherein mechanical surface properties chemical surface properties, electrical surface properties and/or biological surface properties of cells disposed in the biomaterial-based culture system are modulated to change the adhesion of cells disposed in the biomaterial-based culture system, wherein the cells are transfected or transduced with an exogenous nucleic acid or protein for gene activation, gene silencing, gene editing, and/or gene insertion.

21. A composition of matter comprising a cocktail including at least two of: a cytoskeleton inhibitor, an adhesion inhibitor, a TGF-β/Activin pathway inhibitor, and/or a BMP pathway inhibitor,
